(19) United States
(12) Patent Application Publication
(10) Pub. No.: US 2012/0108667 A1
(43) Pub. Date: May 3, 2012

(54) HYDROXY CERAMIDES AND ANALOGS THEREOF AND THEIR USE FOR PREVENTING OR TREATING CANCER

(76) Inventors: Zdzislaw M. Szulc, Charleston, SC (US); Alicja Bielawska, Charleston, SC (US); Yusuf A. Hannun, Sullivans Island, SC (US)

(21) Appl. No.: 13/286,194

(22) Filed: Oct. 31, 2011

Related U.S. Application Data
(60) Provisional application No. 61/408,646, filed on Oct. 31, 2010.

(51) Int. Cl. A61K 31/164; A61P 35/00; C07C 231/24; C07D 319/06; C07C 231/02

(52) U.S. Cl. 514/629; 549/371; 514/625; 564/141; 554/69

(57) ABSTRACT

Disclosed herein are compounds, compositions, methods of treatment and synthetic methods for making compounds related to Ceramides and the use of Ceramides.

### CROSS-REFERENCE TO RELATED APPLICATIONS

This application claims benefit of U.S. Provisional Application No. 61/408,646, filed Oct. 31, 2010. Application No. 61/408,646, filed Oct. 31, 2010, is hereby incorporated herein by reference in its entirety.

### STATEMENT REGARDING FEDERALLY SPONSORED RESEARCH

This invention was made with government support under P01 CA097132-01A and P20 RR017677 awarded by National Institutes of Health (NIH). The government has certain rights in the invention.

### FIELD OF THE INVENTION

The disclosed invention is generally in the field of ceramides and ceramide derivatives, the synthesis of ceramides and ceramide derivatives and use of ceramides and ceramide derivatives.

### BACKGROUND OF THE INVENTION

Ceramides (Cers) have emerged as key modulators of cancer cell growth and apoptosis (L. M. Obeid et al., Science 259 (1993), p. 1769; M. Mimeault, FEBS Lett. 530 (2002), p. 9; A. Carpinteiro et al., Cancer Lett. 264 (2008), p. 1; K. Kitatani, et al., Cell. Signalling 20 (2008), p. 1010). They are a highly diversified structures consisting of different sphingoid base backbones and an amide linked varied chain fatty acids (FAs; mostly C14-C26 saturated, unsaturated, and hydroxylated FAs).

Naturally occurring 2′-hydroxy-Cers (2′-OHCers), namely (2S,3R,2′R,4E)-2′-hydroxy-Cers, contain an additional hydroxyl group located in the alpha position to the carbonyl group in their N-acyl moieties. The formation of an additional chiral center in these molecules gives rise to the possibility to create two stereoisomers: 2′R or 2′S. Natural 2′-OHCers represent (2′R)-isomer (M. Inagaki, et al., Eur. J. Chem. (1998), p. 129; N. Asai, et al., J. Nat. Prod. 64 (2001), p. 1210; Y. Masuda, et al., Biosci. Biotechnol. Biochem. 66 (2002), p. 1531; H. Azuma, et al., J. Org. Chem. 68 (2003), p. 2790; N. Q. Tien, et al., Arch. Pharm. Res. 27 (2004), p. 1020; P. Radhika, et al., Chem. Pharm. Bull. 52 (2004), p. 1345; Y. Masuda, Eur. J. Org. Chem. (2005), p. 4789; F. Leon, et al., J. Med. Chem. 49 (2006), p. 5830; T. Ishii, et al., J. Nat. Prod. 69 (2006), p. 1080).

Recent research showed that sphingolipids (SPLs) containing 2′-OHCers as their building blocks are common components of mammalian and plant specific lipid membranes, cells, organs, and tissues including brain, epidermis and epithelial tissue of the digestive tract (W. M. Holleran, et al., FEBS Lett. 580 (2006), p. 545; A. H. Futerman et al., Nat. Rev. Mol. Cell. Biol. 5 (2004), p. 554; M. Iwamori, Human Cell 18 (2005), p. 117. H. Hama, Biochim. Biophys. Acta 1801 (2010), p. 405) Despite their prevalence in the nervous system, epidermis, and kidney as well as indicated and confirmed contributions to the pathogenesis of many diseases such as Farber's disease, (M. Sugita, et al., J. Lipid Res. 15 (1974), p. 223; M. Iwamori, Clin. Chem. 21 (1975), p. 725) hereditary leukodystrophy (S. Edwardson, et al., Am. J. Hum. Genet. 83 (2008), p. 643) and cancer, (K. Kiguchi, et al., Cancer Sci. 97 (2006), p. 1321; S. A. Richtie, et al., BMC Med. 8 (2010), p. 13) their physiological functions remain mostly unknown (Hama, Biochim. Biophys. Acta 1801 (2010), p. 405).

2′-OH-Sphingomyelin, glucosyl-2′-OH-Cer, lactosyl-2′-OH-Cer, and their complex glyco-SPL analogs have been isolated from different natural sources (Hama, Biochim. Biophys. Acta 1801 (2010), p. 405; K. Karlsson, et al, Biochim. Biophys. Acta 176 (1969), p. 660; O. Nilsson et al., J. Lipid Res. 23 (1982), p. 327; L. Riboni, et al., Eur. J. Biochem. 201 (1992), p. 107). Galactosylceramide, commonly called “cerebroside”, containing cerebronic acid in the N-acyl part of Cer (2′-OH—C24-Cer) is a major glycosphingolipid in brain and nervous tissues and builds up its complex analogs: sulfatide, ganglioside GM4 (sialylgalactosyl-Cer) and Gal2Cer (galabiosyl-Cer) (R. Dahiya, Biochim. Biophys. Acta 875 (1986), p. 220). The ratio of hydroxy- to non-hydroxy-FA in cerebrosides increases with the complexity of the central nervous system (Y. Kishimoto, et al., Mol. Cell Biochem. 254 (1979), p. 1050). This cerebroside was also identified in intestines from the Japanese quail and Runner and Kidney beans (Y. Hirabayashi, et al., Lipids 21 (1986), p. 710; M. Kojima, et al., J. Agric. Food. Chem. 39 (1991), p. 1709).

2′-OHCers are present as sub-components of the skin (Cer 4-7, 9 sub-classes) (W. M. Holleran, et al., FEBS Lett. 580 (2006), p. 545; P. W. Wertz et al., Chem. Phys. Lipids 91 (1998), p. 85; M. Ponec, et al., J. Invest. Dermatol. 120 (2003), p. 581; J. A. Bouwstra, et al., J. Lipid Res. 42 (2001), p. 1759). They are thought to have structural roles to facilitate tight packing of lipids and to influence the conformation of the head groups via the hydrogen bonds (A. Bouwstra, et al., J. Lipid Res. 42 (2001), p. 1759; I. Pascher, Biochem. Biophys. Acta 455 (1976), p. 433; H. Lofgren et al., Chem. Phys. Lipids 20 (1977), p. 273). This class of Cers was also identified in human erythrocytes, the small intestine liver and kidney of rats and the Harderian gland of guinea pigs (H. Hama, Biochim. Biophys. Acta 1801 (2010), p. 405. Article; E. Yasugi, et al, J. Biochem. 110 (1991), p. 202). Some special 2′-OHCers (C3, C16-C26, and C29) were found to have a remarkable biological effects, including cytotoxicity and antiproliferative activities related to activation of caspase 3 (M. Minamino, et al., Microbiology 149 (2003), p. 2071; M. Kyogasima, et al., J. Biochem. 144 (2008), p. 95; H. Azuma, et al., J. Med. Chem. 46 (2003), p. 3445; X. Li, et al., Fitoterapia 78 (2007), p. 490).

2′-OHCers can be produced by the action of fatty acid 2-hydroxylase (FA2H), which has been recently identified molecularly (H. Hama, Biochim. Biophys. Acta 1801 (2010), p. 405; N. L. Alderson, et al., J. Biol. Chem. 279 (2004), p. 48562; Y. Uchida, et al., J. Biol. Chem. 282 (2007), p. 13211). Mutation of this enzyme results in inherited human neurodegenerative disorders (S. Edwardson, et al., Am. J. Hum. Genet. 83 (2008), p. 643; K. J. Dick, et al., Hum. Mutat. 31 (2010), p. E1251).

Deficiency in lysosomal CDase causes a lysosomal storage disease, resulting in the accumulation of 2′-OHCers and Cers in the kidneys, cerebrum, lungs, and stomach of Farber's disease patients (M. Sugita, et al., J. Lipid Res. 15 (1974), p. 223; M. Iwamori et al., Clin. Chem. 21 (1975), p. 725; Y. Sun, et al., Hum. Mol. Genet. 16 (2007), p. 957; J. H. Park et al., Biochem. Biophys. Acta 1758 (2006), p. 2133). Studies performed in certain animal cells showed that 2′-OHCers were preferably converted to galactosylceramides, whereas Cers with normal fatty acids were used for glucosylceramides formation, but this was not a universal rule (P. Van der Bijl, et al., Biochem. J 317 (1996), p. 589). Elevated cellular toxicity and plant growth regulatory activity of some natural 2′-OHCers, were also reported (P. Radhika, et al., Chem. Pharm. Bull. 52 (2004), p. 1345; T. Ishii, et al., J. Nat. Prod. 69 (2006), p. 1080).

The factors that predominantly control the structure of 2′-OHCer or are responsible for its distinct biological functions are not fully understood. However, evidence confirming the roles of 2′-OH group of 2′-OHCers include: (i) formation of the extensive network of hydrogen bonds between the galactose head group of complex 2′-OH-SPL and the polar head of Cer, (ii) promotion of Cers monolayer condensation to a close-packed arrangement, and (iii) increased effects on the phase transition temperatures and gel phases stabilization in comparison to non-hydroxylated analogs (H. Lofgren et al., Chem. Phys. Lipids 20 (1977), p. 273; I. Pascher et al., Chem. Phys. Lipids 20 (1977), p. 175; J. M. Boggs, et al., Biochim. Biophys Acta 938 (1988), p. 361).

Current knowledge on the potential involvement of 2′-OHCers in metabolism and cell signaling phenomena as well as access to advanced structure-biological activity correlation data is very limited due to the lack of well-characterized synthetic 2′-OHCers to be used as molecular probes and analytical standards.

It is therefore an object of the disclosed methods, compounds and compositions herein to provide access and use of varied chain model 2′-OHCers.

### BRIEF SUMMARY OF THE INVENTION

Disclosed herein are compounds, compositions, methods of treatment and synthetic methods for making compounds related to Cers and the use of Cers.

Disclosed are compounds having the structure of formula I;

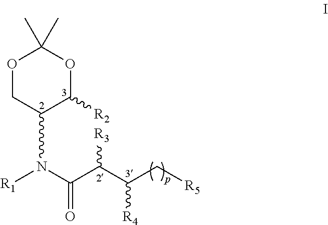

In some forms, R₁ can be H or C₁-C₃ alkyl; R₂ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₃ can be selected from the group consisting of H, hydroxyl, halo, amino, nitro, C₁-C₃ alkoxy and keto; R₄ can be selected from the group consisting of H, hydroxyl, halo, amino, nitro, C₁-C₃ alkoxy and keto; R₅ can be selected from the group consisting of H, hydroxyl, halo and —N⁺-group; and p can be 0-30.

Also disclosed herein are compound made from the method of (a) condensation of a 2-hydroxyl carboxylic acid with an acetonide; and (b) deprotecting the acetonide, wherein the compound has the structure of formula II:

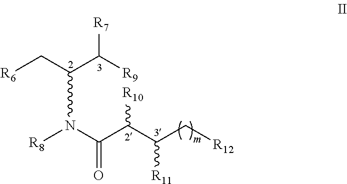

In some forms, R₆ can be selected from the group consisting of H, hydroxyl, C₁-C₃ alkoxy, amino, thio, —OP(O)(OH)₂ and —OP(O)₂OR₁₃NR₁₄; R₁₃ can be C₁-C₃ alkyl; R₁₄ can be (C₁-C₃ alkyl)₃; R₇ can be selected from the group consisting of H, hydroxyl, C₁-C₃ alkoxy and amino; R₈ can be selected from the group consisting of H and C₁-C₃ alkyl; R₉ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₁₀ can be selected from the group consisting of H, hydroxyl, halo, amino, nitro, C₁-C₃ alkoxy and keto; R₁₁ can be selected from the group consisting of H, hydroxyl, halo, amino, nitro, C₁-C₃ alkoxy and keto; R₁₂ can be selected from the group consisting of H, hydroxyl, halo, —N⁺-group; and m can 0-30. The 2-hydroxyl carboxylic acid can be optically pure or a racemic mixture. Thus, for example, disclosed herein are compound made from the method of (a) condensation of a racemic mixture of 2-hydroxyl carboxylic acid with an acetonide; and (b) deprotecting the acetonide. Racemic mixtures are more easily obtain and cheaper to produce. The ability to produce the disclosed compounds while starting with a racemic mixture is an advantage of these forms of the disclosed methods.

In some forms, R₆ can be selected from the group consisting of H, hydroxyl, C₁-C₃ alkoxy, amino, thio, —OP(O)(OH)₂ and —OP(O)₂OR₁₃NR₁₄; R₁₃ can be C₁-C₃ alkyl; R₁₄ can be (C₁-C₃ alkyl)₃; R₇ can be selected from the group consisting of H, hydroxyl; C₁-C₃ alkoxy and amino; R₈ can be selected from the group consisting of H and C₁-C₃ alkyl; R₉ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₁₀ can be selected from the group consisting of H, hydroxyl, halo, amino, nitro, C₁-C₃ alkoxy and keto; R₁₁ can be selected from the group consisting of H, hydroxyl, halo, amino, nitro, C₁-C₃ alkoxy and keto; R₁₂ can be selected from the group consisting of H, hydroxyl, halo, —N⁺-group; and m can 0-30, wherein the following substituents are not simultaneously R₆ is H; R₇ is OH; R₈ is H; R₉ is C₁₅ alkenyl; R₁₀ is OH; R₁₁ is H; R₁₂ is Hand m is 13 or R₆ is H; R₇ is OH; R₈ is H; R₉ is C₁₅ alkenyl or C₁₅ alkyl; R₁₀ is OH; R₁₁ is H; R₁₂ is H and m is 0 or an acceptable salt or ester thereof.

The compounds of formula I and II can by pure isomers or substantially pure isomers, for example, the isomers can be (2S,3R,2′R), (2S,3R,2′S), (2R,3R,2′R), (2R,3R,2′S), (2S,3S,2′R), (2S,3S,2′S), (2R,3S,2′R), (2R,3S,2′S); (2S,3R,3′R), (2S,3R,3′S), (2R,3R,3′R), (2R,3R,3′S), (2S,3S,3′R), (2S,3S,3′S), (2R,3S,3′R) and (2R,3S,3′S).

Also described herein is a method of treating cancer by administering a therapeutically effective amount of a compound of formula II to a subject.

Also disclosed herein is a method of synthesizing a pure isomer by (a) condensation of a 2-hydroxyl carboxylic acid with an acetonide; and (b) isolating pure isomers. Also disclosed herein are compound made from the method of (a) condensation of a racemic mixture of 2-hydroxyl carboxylic acid with an acetonide; and (b) deprotecting the acetonide. The method can also include deprotecting the acetonide.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a 500 MHz NMR spectra of 2′-OHCers and Cers. FIG. 1A shows the comparison of the NMR spectra of 2′-OH—C6-Cer diastereoisomers with C6-Cer in CD3OD solution. Signature shift of the 3-H proton and anisochronism of the polar interface 1Hab/3′-Hab protons. FIG. 1B shows the comparison of (2R′)-, (2′S)-2′-OH—C16-Cers and C16-Cer spectra in CDCl₃ solution. FIG. 1C shows the 1H-1H-COSY spectrum of (2′R)-2′-OH—C6-Cer in CD3OD solution. FIG. 1D shows the 1H-13C—HSQC spectrum of (2′R)-2′-OH—C6-Cer in CD3OD solution.

FIG. 2 shows the inhibitory effects of 2′-OH—C6-Cer and 2′-OHC6-dhCer stereoisomers and corresponding C6-Cer and C6-dhCer on MCF7 cell growth. Concentration-dependent effect at 24 h.

FIG. 3 shows the cellular level of selected analogs in MCF7 cells. FIG. 3A shows the concentration dependent levels at 24 h. FIG. 3B shows the time dependent levels for 10 μM treatments.

FIG. 4 shows the concentration dependent effect of 2′-OH—C6-Cer isomers on endogenous bioactive SPLs for 24 h treatment. FIG. 4A shows the effect on total Cer. FIG. 4B shows the effect on Sph.

FIG. 5 shows the time dependent effects of 10 μM 2′-OH—C6-Cer stereoisomers and C6-Cer on cellular Cers.

FIG. 6 shows the direct conversion of 2′-OH—C6-dhCer stereoisomers and C6-dhCer to the respective unsaturated analogs 2′-OH—C6-Cer and C6-Cer. Concentration dependent effects for 24 h treatments.

FIG. 7 shows the effect of 2′-OH—C6-dhCer stereoisomers and C6-dhCer on cellular SPLs at 24 h. FIG. 7A shows the concentration dependent effects on cellular dhCers. FIG. 7B shows the concentration dependent effects on cellular dhSph. FIG. 7C shows the concentration dependent effects on cellular Cers. FIG. 7D shows the concentration dependent effects on cellular Sph.

FIG. 8 shows the time dependent effects of 10 μM 2′-OH—C6-dhCer diastereoisomers and C6-dhCer on cellular SPLs. FIG. 8A shows the effect on cellular dhCers. FIG. 8B shows the effect on cellular Cers.

FIG. 9 shows the ¹H-¹H COSY NMR of D-e-C6-Ceramide in CD₃OD.

FIG. 10 shows the ¹H-¹H NOESY NMR of ('2R)-2′-Hydroxy-C6-Ceramide in CD₃OD.

FIG. 11 shows the inhibitory effect of 2-OHC6-Cer stereoisomers and C6-Cer on MCF7 breast carcinoma cells at 24 hrs.

FIG. 12 shows concentration dependent cellular levels of C6-Ceramides at 24 hrs.

FIG. 13 shows time dependent cellular levels of C6-Ceramides after 10 μM treatments.

FIG. 14 shows concentration dependent effects of C6-Ceramides on cellular ceramides at 24 hrs.

FIG. 15 shows time dependent effects of C6-Ceramides on cellular ceramides after 10 μM treatments.

FIG. 16 shows the effects of C6-ceramides on cellular ceramide species after 10 μM treatments at 24 hrs.

### DETAILED DESCRIPTION OF THE INVENTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound of formula I is disclosed and discussed and a number of modifications that can be made to a number of molecules are discussed, each and every combination and permutation of those molecules and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

### A. DEFINITIONS

The term “alkyl group” as used herein is a branched or unbranched saturated hydrocarbon group of 1 to 30 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “alkoxy” as used herein is an alkyl group bound through a single, terminal ether linkage; that is, an “alkoxy” group may be defined as —OR where R is alkyl as defined above. A “lower alkoxy” group is an alkoxy group containing from one to six carbon atoms.

The term “alkenyl group” as used herein is a hydrocarbon group of from 2 to 30 carbon atoms and structural formula containing at least one carbon-carbon double bond. Asymmetric structures such as (AB)C═C(CD) are intended to include both the E and Z isomers. This may be presumed in structural formulae herein wherein an asymmetric alkene is present, or it may be explicitly indicated by the bond symbol C. The compounds disclosed in formula I and II herein can be include 4E conformation of the described alkenyl groups.

The term “alkynyl group” as used herein is a hydrocarbon group of 2 to 30 carbon atoms and a structural formula containing at least one carbon-carbon triple bond.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkyl-aryl” and “alkenyl-aryl” as used herein is an aryl group having an alkyl, or alkenyl group as defined above attached to the aromatic group. An example of an alkyl-aryl group is a C₁₃ alkyl-phenyl group. Such phenyl can be substituted with one or more groups including, but not limited to, alkyl, alkynyl, alkenyl, aryl, halide, nitro, amino, ester, keto, aldehyde, hydroxy, carboxylic acid, or alkoxy.

The term “hydroxyalkyl group” as used herein is an alkyl, alkenyl, alkynyl, aryl, aralkyl, cycloalkyl, halogenated alkyl, or heterocycloalkyl group described above that has at least one hydrogen atom substituted with a hydroxyl group. An alkyl-OH group is an alkyl chain terminated with a hydroxyl.

The term “alkoxyalkyl group” is defined as an alkyl, alkenyl, alkynyl, aryl, aralkyl, cycloalkyl, halogenated alkyl, or heterocycloalkyl group described above that has at least one hydrogen atom substituted with an alkoxy group described above.

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

The term “carboxylic acid” as used herein is represented by the formula —C(O)OH.

The term “aldehyde” as used herein is represented by the formula —C(O)H.

The term “keto group” as used herein is represented by the formula —C(O)R, where R is an alkyl, alkenyl, alkynyl, aryl, aralkyl, cycloalkyl, halogenated alkyl, or heterocycloalkyl group described above.

The term “carbonyl group” as used herein is represented by the formula C═O.

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above. The term ‘—N⁺-group” as used herein refers to moiety that includes a positively charged nitrogen. An —N⁺-group also includes salts including a positively charged nitrogen. For example an —N⁺-group can be a pyridium salt.

The term “urethane” as used herein is represented by the formula —OC(O)NRR′, where R and R′ can be, independently, hydrogen, an alkyl, alkenyl, alkynyl, aryl, aralkyl, cycloalkyl, halogenated alkyl, or heterocycloalkyl group described above.

The term “silyl group” as used herein is represented by the formula —SiRR′R″, where R, R′, and R″ can be, independently, hydrogen, an alkyl, alkenyl, alkynyl, aryl, aralkyl, cycloalkyl, halogenated alkyl, alkoxy, or heterocycloalkyl group described above.

The term “sulfo-oxo group” as used herein is represented by the formulas —S(O)₂R, —OS(O)₂R, or, —OS(O)₂OR, where R can be hydrogen, an alkyl, alkenyl, alkynyl, aryl, aralkyl, cycloalkyl, halogenated alkyl, or heterocycloalkyl group described above.

The formulas and methods described herein using the formulas described can, independently, possess two or more of the groups listed above. For example, if R₂ in formula I is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

As used herein, the term “activity” refers to a biological activity.

As used herein, the term “pharmacological activity” refers to the inherent physical properties of a peptide or polypeptide. These properties include but are not limited to half-life, solubility, and stability and other pharmacokinetic properties.

A used herein, a “substantially pure isomer” has at least 70%, 80%, 90%, or 95% of one stereoisomer.

A used herein, a “pure isomer” has at least >95% of one isomer. In some forms, a “pure isomer” can have >96%, >97%. >98% or >%99 of one stereoisomer.

### B. COMPOUNDS

In one aspect described herein are compounds having the formula I. Formula I includes compounds having the structure:

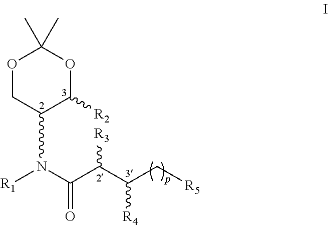

In some forms, R₁ can be H or C₁-C₃ alkyl; R₂ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₃ can be selected from the group consisting of H, hydroxyl, halo, amino, nitro, C₁-C₃ alkoxy and keto; R₄ can be selected from the group consisting of H, hydroxyl, halo, amino, nitro, C₁-C₃ alkoxy and keto; R₅ can be selected from the group consisting of H, hydroxyl, halo and —N⁺-group; and p can be 0-30. In some forms, R₁ can be H or C₁ alkyl. In some forms R₁ can be H. In some forms R₂ can be selected from the group consisting of C₁₀-C₂₀ alkyl, C₁₀-C₂₀ alkenyl, C₁₀-C₂₀ alkynyl, C₁₀-C₂₀ alkyl-aryl, C₁₀-C₂₀ alkenyl-aryl, C₁₀-C₂₀ alkyl-N⁺-group and C₁₀-C₂₀ alkyl-OH. In some forms R₂ can be selected from the group consisting of C₁₅ alkyl, C₁₅ alkenyl, C₁₅ alkynyl, C₁₅ alkyl-aryl, C₁₅ alkenyl-aryl, C₁₅ alkyl-N⁺-group and C₁₅ alkyl-OH. In some forms R₂ can be selected from the group consisting of C₁-C₃₀ alkyl-aryl, C₁-C₃₀ alkenyl-aryl, C₁-C₃₀ alkyl-N⁺-group and C₁-C₃₀ alkyl-OH. In some forms R₂ can be CH═CH—C₁-C₂₉ alkyl. In some forms R₂ can be CH═CH—C₆-C₁₅ alkyl. In some forms R₂ can be CH═CH—C₉ alkyl. In some forms R₃ can be selected from the group consisting of H, hydroxyl, halo and amino. In some forms R₃ can be selected from the group consisting of hydroxyl, halo and amino. In some forms R₃ can be hydroxyl. In some forms R₄ can be selected from the group consisting of H, hydroxyl, halo, amino, and keto. In some forms R₄ can be selected from the group consisting of H or hydroxyl. In some forms R₅ can be selected from the group consisting of H, hydroxyl, and alkyl-N⁺-group. In some forms R₅ can be selected from the group consisting of H, hydroxyl, and alkyl-N⁺-group. In some forms p can be 1-20, 1-10, 2-6, 3-5 or 3. In some forms the alkyl-N⁺-group can be a salt. In some forms the alkyl-N⁺-group can be a pyridinium salt. In some forms R₁ can be H; R₂ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₃ can be hydroxyl,; R₄ can be selected from the group consisting of H, hydroxyl, halo, amino, nitro, C₁-C₃ alkoxy and keto; R₅ can be selected from the group consisting of H, hydroxyl, and —N⁺-group; and p can be 0-30. In some forms the compound can be a pharmaceutically acceptable salt or ester of formula I.

In some forms, R₁ can be H or C₁-C₃ alkyl; R₂ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₃ can be selected from the group consisting of H, hydroxyl, halo, amino, nitro, C₁-C₃ alkoxy and keto; R₄ can be selected from the group consisting of H, hydroxyl, halo, amino, nitro, C₁-C₃ alkoxy and keto; R₅ can be selected from the group consisting of H, hydroxyl, halo and —N⁺-group; and p can be 0-30, wherein the following substituents are not simultaneously R₁ is H; R₂ is C₁₅ alkenyl; R₃ is OH; R₄ is H; R₅ is H and p is 13 or R₁ is H; R₂ is C₁₅ alkenyl or C₁₅ alkyl; is OH; R₄ is H; R₅ is H; and p is 0.

The compounds represented by formula I can be optically active or racemic. In some forms the compounds represented by formula I can be optically active. In some forms the compound is a pure isomer or a substantially pure isomer. In some forms the compound is 2′R. In some forms the compound is 2′S. In some forms the compound can be the an isomer selected from the group consisting of (2S,3R,2′R), (2S,3R,2′S), (2R,3R,2′R), (2R,3R,2′S), (2S,3S,2′R), (2S,3S,2′S), (2R,3S,2′R), (2R,3S,2′S); (2S,3R,3′R), (2S,3R,3′S), (2R,3R,3′R), (2R,3R,3′S), (2S,3S,3′R), (2S,3S,3′S), (2R,3S,3′R) and (2R,3S,3′S).

Any of the compounds of Formula I can be specifically included or excluded. For example, some N-lactylsphingosine and N-lactyldihydrosphingosine analogs described in Azuma, H., et al., J. Med. Chem., 46, (2003), p. 3445, can be specifically included or excluded. In particular, compounds of Formula I where R₂=alkenyl C₁₅H₂₉ or alkyl C₁₅H₃₁; R₁═H, R₃═OH, R₄═H, R₅═H; m=13, and C4′C5′=CH═CH can be specifically included or excluded. Compounds of Formula I where R₂=alkenyl C₁₅H₂₉ or alkyl C₁₅H₃₁; R₁═H, R₃═OH, R₄═H, R₅=none; and m=13 can be specifically included or excluded.

Also described herein are compounds of formula II. In some forms the compounds of formula II can be made by the synthetic methods described herein. Formula II has the structure:

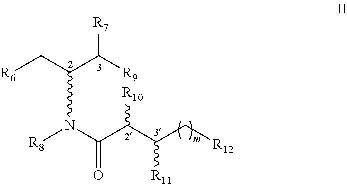

Wherein R₆ can be selected from the group consisting of H, hydroxyl, C₁-C₃ alkoxy, amino, thio, —OP(O)(O)(OH)₂ and —OP(O)₂OR₁₃NR₁₄; R₁₃ can be C₁-C₃ alkyl; R₁₄ can be (C₁-C₃ alkyl)₃; R₇ can be selected from the group consisting of H, hydroxyl, C₁-C₃ alkoxy and amino; R₈ can be selected from the group consisting of H and C₁-C₃ alkyl; R₉ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₁₀ can be selected from the group consisting of H, hydroxyl, halo, amino, nitro, C₁-C₃ alkoxy and keto; R₁₁ can be selected from the group consisting of H, hydroxyl, halo, amino, nitro, C₁-C₃ alkoxy and keto; R₁₂ can be selected from the group consisting of H, hydroxyl, halo, —N⁺-group; and m can 0-30, or a pharmaceutically acceptable salt or ester thereof. In some forms R₆ can be selected from the group consisting of hydroxyl, C₁-C₃ alkoxy, amino, —OP(O)(OH)₂ and —OP(O)₂OR₁₃NR₁₄, wherein R₁₃ is C₂ alkyl and R₁₄ is C₁ alkyl. In some forms R₆ can be selected from the group consisting of hydroxyl, C₁-C₃ alkoxy, —OP(O)(OH)₂ and —OP(O)₂OR₁₃NR₁₄, wherein R₁₃ is C₂ alkyl and R₁₄ is C₁ alkyl. In some forms R₇ can be selected from the group consisting of hydroxyl, C₁ alkoxy and amino. In some forms R₇ can be hydroxyl. In some forms, R₈ can be H or C₁ alkyl. In some forms R₈ can be H. In some forms R₉ can be selected from the group consisting of C₁₀-C₂₀ alkyl, C₁₀-C₂₀ alkenyl, C₁₀-C₂₀ alkynyl, C₁₀-C₂₀ alkyl-aryl, C₁₀-C₂₀ alkenyl-aryl, C₁₀-C₂₀ alkyl-N⁺-group and C₁₀-C₂₀ alkyl-OH. In some forms R₉ can be selected from the group consisting of C₁₅ alkyl, C₁₅ alkenyl, C₁₅ alkynyl, C₁₅ alkyl-aryl, C₁₅ alkenyl-aryl, C₁₅ alkyl-N⁺-group and C₁₅ alkyl-OH. In some forms R₉ can be selected from the group consisting of C₁-C₃₀ alkyl-aryl, C₁-C₃₀ alkenyl-aryl, C₁-C₃₀ alkyl-N⁺-group and C₁-C₃₀ alkyl-OH. In some forms R₉ can be CH═CH—C₁-C₂₉ alkyl. In some forms R₉ can be CH═CH—C₆-C₁₅ alkyl. In some forms R₉ can be CH═CH—C₉ alkyl. In some forms R₁₀ can be selected from the group consisting of H, hydroxyl, halo and amino. In some forms R₁₀ can be selected from the group consisting of hydroxyl, halo and amino. In some forms R₁₀ can be hydroxyl. In some forms R₁₁ can be selected from the group consisting of H, hydroxyl, halo, amino, and keto. In some forms R₁₁ can be selected from the group consisting of H or hydroxyl. In some forms R₁₂ can be selected from the group consisting of H, hydroxyl, and alkyl-N⁺-group. In some forms R₁₂ can be selected from the group consisting of H, hydroxyl, and alkyl-N⁺-group. In some forms m can be 1-20, 1-10, 2-6, 3-5 or 3. In some forms the alkyl-N⁺-group can be a salt. In some forms the alkyl-N⁺-group can be a pyridinium salt.

In some forms, R₆ can be selected from the group consisting of H, hydroxyl, C₁-C₃ alkoxy, amino, thio, —OP(O)(OH)₂ and —OP(O)₂OR₁₃NR₁₄; R₁₃ can be C₁-C₃ alkyl; R₁₄ can be (C₁-C₃ alkyl)₃; R₇ can be selected from the group consisting of H, hydroxyl, C₁-C₃ alkoxy and amino; R₈ can be selected from the group consisting of H and C₁-C₃ alkyl; R₉ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₁₀ can be selected from the group consisting of H, hydroxyl, halo, amino, nitro, C₁-C₃ alkoxy and keto; R₁₁ can be selected from the group consisting of H, hydroxyl, halo, amino, nitro, C₁-C₃ alkoxy and keto; R₁₂ can be selected from the group consisting of H, hydroxyl, halo, —N⁺-group; and m can 0-30, wherein the following substituents are not simultaneously R₆ is H; R₇ is OH; R₈ is H; R₉ is C₁₅ alkenyl; R₁₀ is OH; R₁₁ is H; R₁₂ is Hand m is 13 or R₆ is H; R₇ is OH; R₈ is H; R₉ is C₁₅ alkenyl or C₁₅ alkyl; R₁₀ is OH; R₁₁ is H; R₁₂ is H and m is 0 or an acceptable salt or ester thereof.

In some forms formula II is a synthesized compound. In some forms formula II is a non-natural compound. A non-natural compound is a compound that has been synthetically produced and not merely isolated from a natural source. The compounds represented by formula II can be optically active or racemic. In some forms the compounds represented by formula II can be optically active. In some forms the compound is a pure isomer or a substantially pure isomer. A substantially pure isomer has at least 70%, 80%, 90%, 95% or 99% of one stereoisomer. In some forms the compound is 2′R. In some forms the compound is 2′S. In some forms the compound can be the an isomer selected from the group consisting of (2S,3R,2′R), (2S,3R,2′S), (2R,3R,2′R), (2R,3R,2′S), (2S,3S,2′R), (2S,3S,2′S), (2R,3S,2′R), (2R,3S,2′S); (2S,3R,3′R), (2S,3R,3′S), (2R,3R,3′R), (2R,3R,3′S), (2S,3S,3′R), (2S,3S,3′S), (2R,3S,3′R) and (2R,3S,3′S).

Any of the compounds of Formula II can be specifically included or excluded. For example, compounds of Formula II where R₆═H, R₇═OH, R₉=alkenyl C₁₅H₂₉ or alkyl C₁₅H₃₁; R₈═H, R₁₀═OH, R₁₁═H; m=0, and R₁₂=none can be specifically included or excluded.

Also described herein are pharmaceutically acceptable nontoxic ester, amide, and salt derivatives of those compounds of formula I containing a carboxylic acid moiety.

Formula I also encompasses pharmaceutically acceptable esters, amides, and salts of such compounds, as will be explained in detail, infra.

Such compounds of the formula I and their pharmaceutically acceptable esters, amides, and salts are referred to herein as the inventive compounds.

Formula I also encompasses pharmaceutically acceptable salts. Pharmaceutically acceptable salts are prepared by treating the free acid with an appropriate amount of a pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. The molar ratio of compounds of structural formula (I) to base used are chosen to provide the ratio desired for any particular salts. For preparing, for example, the ammonium salts of the free acid starting material, the starting material can be treated with approximately one equivalent of pharmaceutically acceptable base to yield a neutral salt.

### Uses

The disclosed methods and compositions are applicable to numerous areas including, but not limited to, treatment or reducing risk of diseases treatable by administration of ceramides (Cers) including 2′-hydroxyCers. Theses diseases are know to one skilled in that art and include, for example, cancer. In some forms the disease can be cancer, for example, breast cancer, lung cancer, colon cancer, leukemia. In some forms the cancers can be breast cancer or lung cancer. Other uses are disclosed, apparent from the disclosure, and/or will be understood by those in the art.

Described herein are uses of compounds having the formula II. In one aspect, compounds having the formula II can be used to a method of treating cancer by administering to a subject a therapeutically effective amount of one or more compounds of formula II:

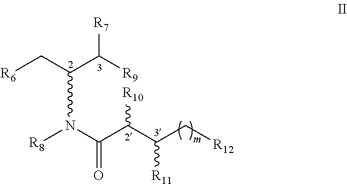

Wherein R₆ can be selected from the group consisting of H, hydroxyl, C₁-C₃ alkoxy, amino, thio, —OP(O)(OH)₂ and —OP(O)₂OR₁₃NR₁₄; R₁₃ can be C₁-C₃ alkyl; R₁₄ can be (C₁-C₃ alkyl)₃; R₇ can be selected from the group consisting of H, hydroxyl, C₁-C₃ alkoxy and amino; R₈ can be selected from the group consisting of H and C₁-C₃ alkyl; R₉ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₁₀ can be selected from the group consisting of H, hydroxyl, halo, amino, nitro, C₁-C₃ alkoxy and keto; R₁₁ can be selected from the group consisting of H, hydroxyl, halo, amino, nitro, C₁-C₃ alkoxy and keto; R₁₂ can be selected from the group consisting of H, hydroxyl, halo, —N⁺-group; and m can 0-30, or a pharmaceutically acceptable salt or ester thereof. In some forms R₆ can be selected from the group consisting of hydroxyl, C₁-C₃ alkoxy, amino, —OP(O)(OH)₂ and —OP(O)₂OR₁₃NR₁₄, wherein R₁₃ is C₂ alkyl and R₁₄ is C₁ alkyl. In some forms R₆ can be selected from the group consisting of hydroxyl, C₁-C₃ alkoxy, —OP(O)(OH)₂ and —OP(O)₂OR₁₃NR₁₄, wherein R₁₃ is C₂ alkyl and R₁₄ is C₁ alkyl. In some forms R₇ can be selected from the group consisting of hydroxyl, C₁ alkoxy and amino. In some forms R₇ can be hydroxyl. In some forms, R₈ can be H or C₁ alkyl. In some forms R₈ can be H. In some forms R₉ can be selected from the group consisting of C₁₀-C₂₀ alkyl, C₁₀-C₂₀ alkenyl, C₁₀-C₂₀ alkynyl, C₁₀-C₂₀ alkyl-aryl, C₁₀-C₂₀ alkenyl-aryl, C₁₀-C₂₀ alkyl-N⁺-group and C₁₀-C₂₀ alkyl-OH. In some forms R₉ can be selected from the group consisting of C₁₅ alkyl, C₁₅ alkenyl, C₁₅ alkynyl, C₁₅ alkyl-aryl, C₁₅ alkenyl-aryl, C₁₅ alkyl-N⁺-group and C₁₅ alkyl-OH. In some forms R₉ can be selected from the group consisting of C₁-C₃₀ alkyl-aryl, C₁-C₃₀ alkenyl-aryl, C₁-C₃₀ alkyl-N⁺-group and C₁-C₃₀ alkyl-OH. In some forms R₉ can be CH═CH—C₁-C₂₉ alkyl. In some forms R₉ can be CH═CH—C₆-C₁₅ alkyl. In some forms R₉ can be CH═CH—C₉ alkyl. In some forms R₁₀ can be selected from the group consisting of H, hydroxyl, halo and amino. In some forms R₁₀ can be selected from the group consisting of hydroxyl, halo and amino. In some forms R₁₀ can be hydroxyl. In some forms R₁₁ can be selected from the group consisting of H, hydroxyl, halo, amino, and keto. In some forms R₁₁ can be selected from the group consisting of H or hydroxyl. In some forms R₁₂ can be selected from the group consisting of H, hydroxyl, and alkyl-N⁺-group. In some forms R₁₂ can be selected from the group consisting of H, hydroxyl, and alkyl-N⁺-group. In some forms m can be 1-20, 1-10, 2-6, 3-5 or 3. In some forms the alkyl-N⁺-group can be a salt. In some forms the alkyl-N⁺-group can be a pyridinium salt.

In some forms, R₆ can be selected from the group consisting of H, hydroxyl, C₁-C₃ alkoxy, amino, thio, —OP(O)(OH)₂ and —OP(O)₂OR₁₃NR₁₄; R₁₃ can be C₁-C₃ alkyl; R₁₄ can be (C₁-C₃ alkyl)₃; R₇ can be selected from the group consisting of H, hydroxyl, C₁-C₃ alkoxy and amino; R₈ can be selected from the group consisting of H and C₁-C₃ alkyl; R₉ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₁₀ can be selected from the group consisting of H, hydroxyl, halo, amino, nitro, C₁-C₃ alkoxy and keto; R₁₁ can be selected from the group consisting of H, hydroxyl, halo, amino, nitro, C₁-C₃ alkoxy and keto; R₁₂ can be selected from the group consisting of H, hydroxyl, halo, —N⁺-group; and m can 0-30, wherein the following substituents are not simultaneously R₆ is H; R₇ is OH; R₈ is H; R₉ is C₁₅ alkenyl; R₁₀ is OH; R₁₁ is H; R₁₂ is Hand m is 13 or R₆ is H; R₇ is OH; R₈ is H; R₉ is C₁₅ alkenyl or C₁₅ alkyl; R₁₀ is OH; R₁₁ is H; R₁₂ is H and m is 0 or an acceptable salt or ester thereof.

In some forms formula II is a synthesized compound. In some forms formula II is a non-natural compound. A non-natural compound is a compound that has been synthetically produced and not merely isolated from a natural source. The compounds represented by formula II can be optically active or racemic. In some forms the compounds represented by formula II can be optically active. In some forms the compound is a pure isomer or a substantially pure isomer. A substantially pure isomer has at least 70%, 80%, 90%, 95% or 99% of one stereoisomer. In some forms the compound is 2′R. In some forms the compound is 2′S. In some forms the compound can be the an isomer selected from the group consisting of (2S,3R,2′R), (2S,3R,2′S), (2R,3R,2′R), (2R,3R,2′S), (2S,3S,2′R), (2S,3S,2′S), (2R,3S,2′R), (2R,3S,2′S); (2S,3R,3′R), (2S,3R,3′S), (2R,3R,3′R), (2R,3R,3′S), (2S,3S,3′R), (2S,3S,3′S), (2R,3S,3′R) and (2R,3S,3′S).

In some forms of the methods the subject has been diagnosed with a disease treatable by administration of ceramides (Cers) including 2′-hydroxyCers. In some forms the subject has been diagnosed with cancer, for example, breast cancer, lung cancer, leukemia or colon cancer. In some forms the subject is administered an effective amount of formula II to treat a disease treatable by administration of ceramides (Cers) including 2′-hydroxyCers.

Formula II can also be administered in a composition. In some forms, the composition can include other agents, such as, therapeutic, diagnostic or prophylactic agents, for example, anti-cancer drugs. The composition can also include one or more excipients known in the art.

In some forms methods for the simultaneous preparation of pure stereoisomers of 2′-hydroxy-bioactive amides, namely 2′-hydroxy-ceramides and analogs possessing an additional chiral center in their N-acyl parts from chiral aminoalcohol acetonides and known racemic mixtures of 2′-hydroxy fatty acids. Accordingly to the protocol, the methods produce the desired compounds in a two-step synthetic process. The obtained 2′-hydroxy-ceramide/dihydroceramide stereoisomers showed higher antiproliferative activities and stereospecific cellular responses as compared to their nonhydroxylated analogs.

Any of the compounds of Formula II can be specifically included or excluded. For example, some N-lactylsphingosine and N-lactyldihydrosphingosine analogs described in Azuma, H., et al., J. Med. Chem., 46, (2003), p. 3445, can be specifically included or excluded. In particular, compounds of Formula II where R₆═H, R₇═OH, R₉=alkenyl C₁₅H₂₉ or alkyl C₁₅H₃₁; R₃═H, R₁₀═OH, R₁₁═H; m=0, and R₁₂=none can be specifically included or excluded.

These above compounds have anticancer activity/were tested in HL-60 cells. Also, a few naturally occurring very long chains 2-OH-cers are known to have anticancer activity: Leon, F., J. med. Chem., 49, (2006), p. 5830; Minamino, M., Microbiology, 149, (2003), p. 2071; Kyogashima, M., J. Biochem., 144, (2008), p. 95; and Li, X., Fitoterapia, 78, (2007), p. 490. Their isolation and reported methods of re-synthesis are tedious, multi-step and low efficient processes.

### Methods

### A. Synthetic Methods

Described herein are methods for making compounds having the formula I and formula II. In one aspect, a method of making the compounds having the formula II involves (a) condensation of a 2-hydroxyl carboxylic acid with an acetonide; and (b) deprotecting the acetonide. In one aspect, a method of making the compounds having the formula I involves (a) condensation of a 2-hydroxyl carboxylic acid with an acetonide. In some forms the synthetic method can further include an isolation step after step (a). In some forms the isolation step is performed by chromatography, such as column chromatography. In some forms the condensation is a catalyzed condensation reaction. For example, the condensation reaction can be performed using dicyclohexylcarbodiimide (DCC), N-hydroxysuccinimide (NHS), and 1-hydroxybenzotriazole (HOBt) in ethylene glycol dimethyl ether (EGDE) solution. In some forms the deprotection reaction can be made by TsOH. The 2-hydroxyl carboxylic acid can be optically pure or a racemic mixture. Thus, for example, disclosed herein are compound made from the method of (a) condensation of a racemic mixture of 2-hydroxyl carboxylic acid with an acetonide; and (b) deprotecting the acetonide. Racemic mixtures are more easily obtain and cheaper to produce. The ability to produce the disclosed compounds while starting with a racemic mixture is an advantage of these forms of the disclosed methods.

In some forms the 2-hydroxyl carboxylic acid is a racemic mixture of the 2-hydroxyl carboxylic acid. In some forms the some forms the 2-hydroxyl carboxylic acid has the structure:

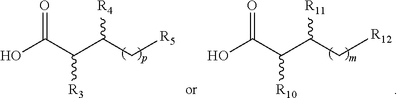

In some forms the acetonide has the structure

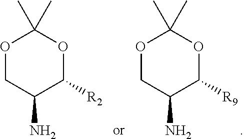

R₉ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₁₀ can be selected from the group consisting of H, hydroxyl, halo, amino, nitro, C₁-C₃ alkoxy and keto; R₁₁ can be selected from the group consisting of H, hydroxyl, halo, amino, nitro, C₁-C₃ alkoxy and keto; R₁₂ can be selected from the group consisting of H, hydroxyl, halo, —N⁺-group; and m can 0-30, or a pharmaceutically acceptable salt or ester thereof. In some forms R₉ can be selected from the group consisting of C₁₀-C₂₀ alkyl, C₁₀-C₂₀ alkenyl, C₁₀-C₂₀ alkynyl, C₁₀-C₂₀ alkyl-aryl, C₁₀-C₂₀ alkenyl-aryl, C₁₀-C₂₀ alkyl-N⁺-group and C₁₀-C₂₀ alkyl-OH. In some forms R₉ can be selected from the group consisting of C₁₅ alkyl, C₁₅ alkenyl, C₁₅ alkynyl, C₁₅ alkyl-aryl, C₁₅ alkenyl-aryl, C₁₅ alkyl-N⁺-group and C₁₅ alkyl-OH. In some forms R₉ can be selected from the group consisting of C₁-C₃₀ alkyl-aryl, C₁-C₃₀ alkenyl-aryl, C₁-C₃₀ alkyl-N⁺-group and C₁-C₃₀ alkyl-OH. In some forms R₉ can be CH═CH—C₁-C₂₉ alkyl. In some forms R₉ can be CH═CH—C₆-C₁₅ alkyl. In some forms R₉ can be CH═CH—C₉ alkyl. In some forms R₁₀ can be selected from the group consisting of H, hydroxyl, halo and amino. In some forms R₁₀ can be selected from the group consisting of hydroxyl, halo and amino. In some forms R₁₀ can be hydroxyl. In some forms R₁₁ can be selected from the group consisting of H, hydroxyl, halo, amino, and keto. In some forms R₁₁ can be selected from the group consisting of H or hydroxyl. In some forms R₁₂ can be selected from the group consisting of H, hydroxyl, and alkyl-N⁺-group. In some forms R₁₂ can be selected from the group consisting of H, hydroxyl, and alkyl-N⁺-group.

In some forms, R₂ can be selected from the group consisting of C₁-C₃₀ alkyl, C₁-C₃₀ alkenyl, alkynyl, C₁-C₃₀ alkyl-aryl, C₂-C₃₀ alkenyl-aryl, C₁-C₃₀ alkyl-N⁺-group and C₁-C₃₀ alkyl-OH; R₃ can be selected from the group consisting of H, hydroxyl, halo, amino, nitro, C₁-C₃ alkoxy and keto; R₄ can be selected from the group consisting of H, hydroxyl, halo, amino, nitro, C₁-C₃ alkoxy and keto; R₅ can be selected from the group consisting of H, hydroxyl, halo and —N⁺-group; and p can be 0-30. In some forms R₂ can be selected from the group consisting of C₁₀-C₂₀ alkyl, C₁₀-C₂₀ alkenyl, C₁₀-C₂₀ alkynyl, C₁₀-C₂₀ alkyl-aryl, C₁₀-C₂₀ alkenyl-aryl, alkyl-N⁺-group and C₁₀-C₂₀ alkyl-OH. In some forms R₂ can be selected from the group consisting of C₁₅ alkyl, C₁₅ alkenyl, C₁₅ alkynyl, C₁₅ alkyl-aryl, C₁₅ alkenyl-aryl, C₁₅ alkyl-N⁺-group and C₁₅ alkyl-OH. In some forms R₂ can be selected from the group consisting of C₁-C₃₀ alkyl-aryl, C₁-C₃₀ alkenyl-aryl, C₁-C₃₀ alkyl-N⁺-group and C₁-C₃₀ alkyl-OH. In some forms R₂ can be CH═CH—C₁-C₂₉ alkyl. In some forms R₂ can be CH═CH—C₆-C₁₅ alkyl. In some forms R₂ can be CH═CH—C₉ alkyl. In some forms R₃ can be selected from the group consisting of H, hydroxyl, halo and amino. In some forms R₃ can be selected from the group consisting of hydroxyl, halo and amino. In some forms R₃ can be hydroxyl. In some forms R₄ can be selected from the group consisting of H, hydroxyl, halo, amino, and keto. In some forms R₄ can be selected from the group consisting of H or hydroxyl. In some forms R₅ can be selected from the group consisting of H, hydroxyl, and alkyl-N⁺-group. In some forms R₅ can be selected from the group consisting of H, hydroxyl, and alkyl-N⁺-group. In some forms p can be 1-20, 1-10, 2-6, 3-5 or 3. In some forms the alkyl-N⁺-group can be a salt. In some forms the alkyl-N⁺-group can be a pyridinium salt. In some forms R₂ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₃ can be hydroxyl,; R₄ can be selected from the group consisting of H, hydroxyl, halo, amino, nitro, C₁-C₃ alkoxy and keto; R₅ can be selected from the group consisting of H, hydroxyl, and —N⁺-group; and p can be 0-30.

The pyriduium salts described herein can be made using 2 steps which are known procedure. The first step is described by Rai et al. (Org. Lett. 6(17): 2861 (2004). Here, 2′-OH-Cer exchanges its olefinic sphingosine backbone tail with other alken, namely, 11-bromo-1-undecene, in a reaction known as olefin metathesis. In the second step, which is described by Szulc et al. (Bioorg. Med. Chem. 14(21) 7083-104 (2006), the formed omega-bromo-2′OH-Cer intermediate is reacting with pyridine in toluene solution at elevated temperature.

The synthesized compound made by the methods described herein can have the structure of formula I:

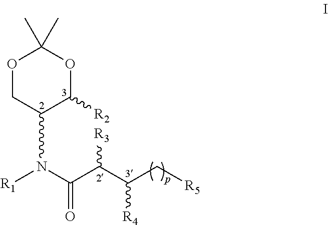

In some forms, R₁ can be H or C₁-C₃ alkyl; R₂ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₃ can be selected from the group consisting of H, hydroxyl, halo, amino, nitro, C₁-C₃ alkoxy and keto; R₄ can be selected from the group consisting of H, hydroxyl, halo, amino, nitro, C₁-C₃ alkoxy and keto; R₅ can be selected from the group consisting of H, hydroxyl, halo and —N⁺-group; and p can be 0-30. In some forms, R₁ can be H or C₁ alkyl. In some forms R₁ can be H. In some forms R₂ can be selected from the group consisting of C₁₀-C₂₀ alkyl, C₁₀-C₂₀ alkenyl, C₁₀-C₂₀ alkynyl, C₁₀-C₂₀ alkyl-aryl, C₁₀-C₂₀ alkenyl-aryl, C₁₀-C₂₀ alkyl-N⁺-group and C₁₀-C₂₀ alkyl-OH. In some forms R₂ can be selected from the group consisting of C₁₅ alkyl, C₁₅ alkenyl, C₁₅ alkynyl, C₁₅ alkyl-aryl, C₁₅ alkenyl-aryl, C₁₅ alkyl-N⁺-group and C₁₅ alkyl-OH. In some forms R₂ can be selected from the group consisting of C₁-C₃₀ alkyl-aryl, C₁-C₃₀ alkenyl-aryl, C₁-C₃₀ alkyl-N⁺-group and C₁-C₃₀ alkyl-OH. In some forms R₂ can be CH═CH—C₁-C₂₉ alkyl. In some forms R₂ can be CH═CH—C₆-C₁₅ alkyl. In some forms R₂ can be CH═CH—C₉ alkyl. In some forms R₃ can be selected from the group consisting of H, hydroxyl, halo and amino. In some forms R₃ can be selected from the group consisting of hydroxyl, halo and amino. In some forms R₃ can be hydroxyl. In some forms R₄ can be selected from the group consisting of H, hydroxyl, halo, amino, and keto. In some forms R₄ can be selected from the group consisting of H or hydroxyl. In some forms R₅ can be selected from the group consisting of H, hydroxyl, and alkyl-N⁺-group. In some forms R₅ can be selected from the group consisting of H, hydroxyl, and alkyl-N⁺-group. In some forms p can be 1-20, 1-10, 2-6, 3-5 or 3. In some forms the alkyl-N⁺-group can be a salt. In some forms the alkyl-N⁺-group can be a pyridinium salt. In some forms R₁ can be H; R₂ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₃ can be hydroxyl,; R₄ can be selected from the group consisting of H, hydroxyl, halo, amino, nitro, C₁-C₃ alkoxy and keto; R₅ can be selected from the group consisting of H, hydroxyl, and —N⁺-group; and p can be 0-30. In some forms the compound can be a pharmaceutically acceptable salt or ester of formula I.

In some forms, R₁ can be H or C₁-C₃ alkyl; R₂ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₃ can be selected from the group consisting of H, hydroxyl, halo, amino, nitro, C₁-C₃ alkoxy and keto; R₄ can be selected from the group consisting of H, hydroxyl, halo, amino, nitro, C₁-C₃ alkoxy and keto; R₅ can be selected from the group consisting of H, hydroxyl, halo and —N⁺-group; and p can be 0-30, wherein the following substituents are not simultaneously R₁ is H; R₂ is C₁₅ alkenyl; R₃ is OH; R₄ is H; R₅ is H and p is 13 or R₁ is H; R₂ is C₁₅ alkenyl or C₁₅ alkyl; is OH; R₄ is H; R₅ is H; and p is 0.

The compounds represented by formula I can be optically active or racemic. In some forms the compounds represented by formula I can be optically active. In some forms the compound is a pure isomer or a substantially pure isomer. In some forms the compound is 2′R. In some forms the compound is 2′S. In some forms the compound can be the an isomer selected from the group consisting of (2S,3R,2′R), (2S,3R,2′S), (2R,3R,2′R), (2R,3R,2′S), (2S,3S,2′R), (2S,3S,2′S), (2R,3S,2′R), (2R,3S,2′S); (2S,3R,3′R), (2S,3R,3′S), (2R,3R,3′R), (2R,3R,3′S), (2S,3S,3′R), (2S,3S,3′S), (2R,3S,3′R) and (2R,3S,3′S).

Any of the compounds of Formula I can be specifically included or excluded. For example, compounds of Formula I where R₂=alkenyl C₁₅H₂₉ or alkyl C₁₅H₃₁; R₁═H, R₃═OH, R₄═H, R₅═H; m=13, and C4′C5′=CH═CH can be specifically included or excluded. Compounds of Formula I where R₂=alkenyl C₁₅H₂₉ or alkyl C₁₅H₃₁; R₁═H, R₃═OH, R₄═H, R₅=none; and m=13 can be specifically included or excluded.

The synthesized compound made by the methods described herein can have the structure of formula II:

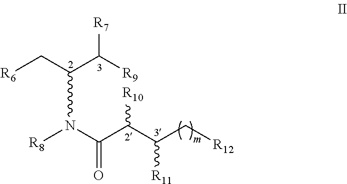

In some forms, R₆ can be selected from the group consisting of H, hydroxyl, C₁-C₃ alkoxy, amino, thio, —OP(O)(OH)₂ and —OP(O)₂OR₁₃NR₁₄; R₁₃ can be C₁-C₃ alkyl; R₁₄ can be (C₁-C₃ alkyl)₃; R₇ can be selected from the group consisting of H, hydroxyl, C₁-C₃ alkoxy and amino; R₈ can be selected from the group consisting of H and C₁-C₃ alkyl; R₉ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₁₀ can be selected from the group consisting of H, hydroxyl, halo, amino, nitro, C₁-C₃ alkoxy and keto; R₁₁ can be selected from the group consisting of H, hydroxyl, halo, amino, nitro, C₁-C₃ alkoxy and keto; R₁₂ can be selected from the group consisting of H, hydroxyl, halo, —N⁺-group; and m can 0-30. In some forms R₆ can be selected from the group consisting of hydroxyl, C₁-C₃ alkoxy, amino, —OP(O)(OH)₂ and —OP(O)₂OR₁₃NR₁₄, wherein R₁₃ is C₂ alkyl and R₁₄ is C₁ alkyl. In some forms R₆ can be selected from the group consisting of hydroxyl, C₁-C₃ alkoxy, —OP(O)(OH)₂ and —OP(O)₂OR₁₃NR₁₄, wherein R₁₃ is C₂ alkyl and R₁₄ is C₁ alkyl. In some forms R₇ can be selected from the group consisting of hydroxyl, C₁ alkoxy and amino. In some forms R₇ can be hydroxyl. In some forms, R₈ can be H or C₁ alkyl. In some forms R₈ can be H. In some forms R₉ can be selected from the group consisting of C₁₀-C₂₀ alkyl, C₁₀-C₂₀ alkenyl, C₁₀-C₂₀ alkynyl, C₁₀-C₂₀ alkyl-aryl, C₁₀-C₂₀ alkenyl-aryl, C₁₀-C₂₀ alkyl-N⁺-group and C₁₀-C₂₀ alkyl-OH. In some forms R₉ can be selected from the group consisting of C₁₅ alkyl, C₁₅ alkenyl, C₁₅ alkynyl, C₁₅ alkyl-aryl, C₁₅ alkenyl-aryl, C₁₅ alkyl-N⁺-group and C₁₅ alkyl-OH. In some forms R₉ can be selected from the group consisting of C₁-C₃₀ alkyl-aryl, C₁-C₃₀ alkenyl-aryl, C₁-C₃₀ alkyl-N⁺-group and C₁-C₃₀ alkyl-OH. In some forms R₉ can be CH═CH—C₁-C₂₉ alkyl. In some forms R₉ can be CH═CH—C₆-C₁₅ alkyl. In some forms R₉ can be CH═CH—C₉ alkyl. In some forms R₁₀ can be selected from the group consisting of H, hydroxyl, halo and amino. In some forms R₁₀ can be selected from the group consisting of hydroxyl, halo and amino. In some forms R₁₀ can be hydroxyl. In some forms R₁₁ can be selected from the group consisting of H, hydroxyl, halo, amino, and keto. In some forms R₁₁ can be selected from the group consisting of H or hydroxyl. In some forms R₁₂ can be selected from the group consisting of H, hydroxyl, and alkyl-N⁺-group. In some forms R₁₂ can be selected from the group consisting of H, hydroxyl, and alkyl-N⁺-group. In some forms m can be 1-20, 1-10, 2-6, 3-5 or 3. In some forms the alkyl-N⁺-group can be a salt. In some forms the alkyl-N⁺-group can be a pyridinium salt.

In some forms, R₆ can be selected from the group consisting of H, hydroxyl, C₁-C₃ alkoxy, amino, thio, —OP(O)(OH)₂ and —OP(O)₂OR₁₃NR₁₄; R₁₃ can be C₁-C₃ alkyl; R₁₄ can be (C₁-C₃ alkyl)₃; R₇ can be selected from the group consisting of H, hydroxyl, C₁-C₃ alkoxy and amino; R₈ can be selected from the group consisting of H and C₁-C₃ alkyl; R₉ can be selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH; R₁₀ can be selected from the group consisting of H, hydroxyl, halo, amino, nitro, C₁-C₃ alkoxy and keto; R₁₁ can be selected from the group consisting of H, hydroxyl, halo, amino, nitro, C₁-C₃ alkoxy and keto; R₁₂ can be selected from the group consisting of H, hydroxyl, halo, —N⁺-group; and m can 0-30, wherein the following substituents are not simultaneously R₆ is H; R₇ is OH; R₈ is H; R₉ is C₁₅ alkenyl; R₁₀ is OH; R₁₁ is H; R₁₂ is Hand m is 13 or R₆ is H; R₇ is OH; R₈ is H; R₉ is C₁₅ alkenyl or C₁₅ alkyl; R₁₀ is OH; R₁₁ is H; R₁₂ is H and m is 0 or an acceptable salt or ester thereof.

In some forms formula II is a synthesized compound. In some forms formula II is a non-natural compound. A non-natural compound is a compound that has been synthetically produced and not merely isolated from a natural source. The compounds represented by formula II can be optically active or racemic. In some forms the compounds represented by formula II can be optically active. In some forms the compound is a pure isomer or a substantially pure isomer. A substantially pure isomer has at least 70%, 80%, 90%, 95% or 99% of one stereoisomer. In some forms the compound is 2′R. In some forms the compound is 2′S. In some forms the compound can be the an isomer selected from the group consisting of (2S,3R,2′R), (2S,3R,2′S), (2R,3R,2′R), (2R,3R,2′S), (2S,3S,2′R), (2S,3S,2′S), (2R,3S,2′R), (2R,3S,2′S); (2S,3R,3′R), (2S,3R,3′S), (2R,3R,3′R), (2R,3R,3′S), (2S,3S,3′R), (2S,3S,3′S), (2R,3S,3′R) and (2R,3S,3′S).

Any of the compounds of Formula II can be specifically included or excluded. For example, compounds of Formula II where R₆═H, R₇═OH, R₉=alkenyl C₁₅H₂₉ or alkyl C₁₅H₃₁; R₈═H, R₁₀═OH, R₁₁═H; m=0, and R₁₂=none can be specifically included or excluded.

The scheme below is a general synthetic scheme of the synthesis of exemplary compounds encompassed by formula I and II. All different substitutes, represented by R groups, in formula I and II can be incorporated into this synthetic scheme.

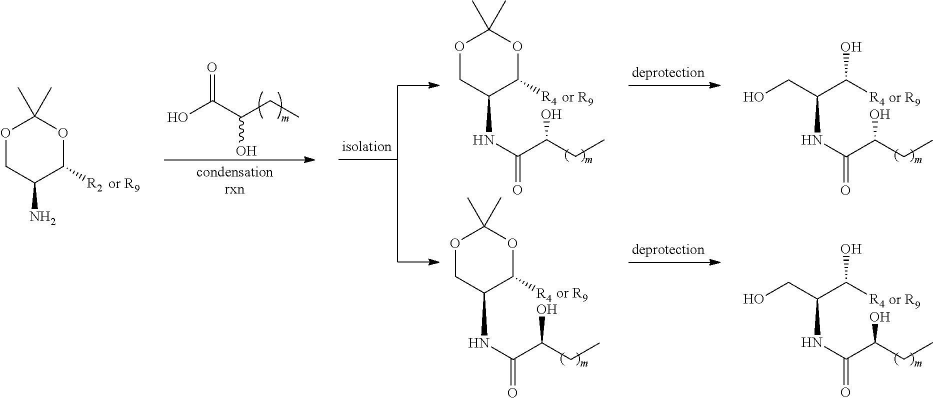

Provided is also a synthetic process for the preparation of pure stereoisomers of 2′ hydroxy bioactive amides, namely ceramides and analogs. In some forms, of the process start from accessible acetonide derivatives of aminoalcohols (e.g. sphingoid bases) and utilizes known reagents (known amide bond forming reagents and racemic mixtures of 2-hydroxy-fatty acids or their analogs.) In some forms, the methods provide access to pure stereoisomers of bioactive sphingolipids (e.g. 2-hydroxy-ceramides) possessing an additional chiral center in their N-acyl parts. These compounds can be used as molecular probes, analytical standards, or anticancer agents. In some forms, of the process can have broad application and can be capable of delivering separable sets of any kind of lipophilic amide diastereoisomers possessing an additional chiral center in any position of their N-acyl part (1,3-O-acetonide protection groups as an efficient separation handle of the diastereomeric mixtures of bioactive amides/ceramides/analogs).

In some forms of the synthetic process can be applied to the preparation of other bioactive sphingolipids and their analogs namely pyridinium ceramides possessing their N-acyl part and additional hydroxyl group or other substituent.

### B. Administration

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “monitoring” as used herein refers to any method in the art by which an activity can be measured.

The term “providing” as used herein refers to any means of adding a compound or molecule to something known in the art. Examples of providing can include the use of pipettes, pipetmans, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the compounds of the invention.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitiative terms. Thus, for example, characteristics or features of a disease, pathological condition, or disorder and/or symptoms of a disease, pathological condition, or disorder can be reduced to any effect or to any amount.

A cell can be in vitro. Alternatively, a cell can be in vivo and can be found in a subject. A “cell” can be a cell from any organism including, but not limited to, a bacterium.

In one aspect, the compounds described herein can be administered to a subject comprising a human or an animal including, but not limited to, a mouse, dog, cat, horse, bovine or ovine and the like, that is in need of alleviation or amelioration from a recognized medical condition.

By the term “effective amount” or “pharmaceutically effective amount” of a compound as provided herein is meant a nontoxic but sufficient amount of the compound to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The efficacy of administration of a particular dose of the compounds or compositions according to the methods described herein can be determined by evaluating the particular aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

By “pharmaceutically acceptable” is meant a material that is not biologically or otherwise undesirable, i.e., the material can be administered to an individual along with the selected compound without causing any undesirable biological effects or interacting in a deleterious manner with any of the other components of the pharmaceutical composition in which it is contained.

Any of the compounds having the formula II can be used therapeutically in combination with a pharmaceutically acceptable carrier. The compounds described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

The pharmaceutical compositions described herein can include, but are not limited to, carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The compounds and pharmaceutical compositions described herein can be administered to the subject in a number of ways depending on whether local or systemic treatment is desired, and on the area to be treated. Thus, for example, a compound or pharmaceutical composition described herein can be administered as an ophthalmic solution and/or ointment to the surface of the eye. Moreover, a compound or pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

### EXAMPLES

### Cell Culture

MCF7 cells (breast adenocarcinoma, pleural effusion) were purchased from American type Culture Collection (ATCC, Rockville, Md., USA) and grown in RPMI 1640 media (Life Technologies, Inc) supplemented with 10% fetal calf serum (FCS, Summit Biotechnology, CO, USA) and maintained under standard incubator conditions (humidified atmosphere 95% air, 5% CO2, 37° C.). A parallel set of cells was used to determine cell proliferation and to prepare lipid extracts for MS analysis.

Cell Proliferation

MCF7 cells were seeded at a density of ˜50% (corresponding to 1×106 cells) in 10 ml of 10% FCS and, after an overnight incubation, were treated with the compounds at (0-50 μM) in ethanol (<0.1%) and the changes in cell numbers after 24 h were determined and expressed as a percentage of the untreated controls. Briefly, media were removed, cells were washed twice with PBS, detached using 1% Trypsin and centrifuged at 800 rpm. Cell pellets were resuspended in PBS and Trypan blue (Sigma Chemicals, St. Louis, Mo., USA) was added (1:1 dilution). Under a light microscope, the percentage of unstained and stained cells was assessed. The IC₅₀ represents the drug concentration resulting in 50% growth inhibition compared to control cells.

LC-MS/MS Analysis of Endogenous SPLs and Cellular Levels of Exogenously Added Compounds

Advanced analyses were performed on a Thermo Finnigan TSQ 7000, triple-stage quadrupole mass spectrometer operating in a Multiple Reaction Monitoring (MRM) positive ionization mode as described (J. Bielawski, et al., Methods 39 (2006), p. 82; J. Bielawski, et al., Methods Mol. Biol. 579 (2009), p. 443). Quantitative analysis of the cellular level of 2′-OH—C6-Cers/dhCers and C6-Cer/dhCer was based on the calibration curves generated by spiking an artificial matrix with known amounts of target standards and an equal amount of the internal standard (IS). The target analyte to IS peak area ratios from the samples were similarly normalized to their respective IS and compared to the calibration curves using a linear regression model. Final results were expressed as the level of the particular SPLs/phospholipids (Pi) determined from the Bligh & Dyer lipid extract and expressed as SPLs/Pi (pmol/nmol).

### 1. Example 1

### Synthesis of Pure Isomers of Cers

A straightforward method for the simultaneous preparation of (2S,3R,2′R)- and (2S,3R,2′S)-2′-hydroxy-ceramides (2′-OHCer) from (2S,3R)-sphingosine acetonide precursors and racemic mixtures of 2-hydroxy fatty acids (2-OHFAs) is described. The obtained 2′-OH—C4-, —C6-, —C12-, —C16-Cer and 2′-OH—C6-dhCer pairs of diastereoisomers were characterized thoroughly by TLC, MS, NMR, and optical rotation. Dynamic and multidimensional NMR studies provided evidence that polar interfaces of 2′-OHCers are extended and more rigid than observed for the corresponding non-hydroxylated analogs. Stereospecific profile on growth suppression of MCF7 cells was observed for (2′R)- and (2′S)-2′-OH—C6-Cers and their dihydro analogs. The (2′R)-isomers were more active than the (2′S)-isomers (IC₅₀˜3 μM/8 μM and IC₅₀˜8 μM/12 μM, respectively), surpassing activity of the ordinary C6-Cer (IC₅₀˜12 μM) and C6-dhCer (IC₅₀˜38 μM). Neither isomer of 2′-OH—C6-Cers and 2′-OH—C6-dhCers was metabolized to their cellular long chain 2′-OH-homologs. Surprisingly, the most active (2′R)-isomers did not influence the levels of the cellular Cers nor dhCers. Contrary to this, the (2′S)-isomers generated cellular Cers and dhCers efficiently. In comparison, the ordinary C6-Cer and C6-dhCer also significantly increased the levels of their cellular long chain homologs. These peculiar anabolic responses and SAR data indicate that (2′R)-2′-OHCers/dhCers may interact with some distinct cellular regulatory targets in a specific and more effective manner than their non-hydroxylated analogs. Thus, stereoisomers of 2′-OHCers can be potentially utilized as novel molecular tools to study lipid-protein interactions, cell signaling phenomena and to understand the role of hydroxylated sphingolipids in cancer biology, pathogenesis and therapy.

Examples of model amides, i.e. 2′R and 2′S-2-hydroxy-C6-cermides and corresponding dihydroceramides showed higher anticancer activity in MCF7 cells than their non-hydroxylated analog C6-ceramide (IC50=3 μM/8 μM versus 12 μM).

Although ceramide has been extensively studied as a mediator of apoptotic responses, 2′-OHCers have never been considered as ubiquitous signaling molecules. However, the preliminary studies showed that 2′-OHCers were ubiquitously present at a very low level in numerous cancer cell types (e.g., ˜5% of total Cers in MCF7 cells, unpublished) suggesting that these Cers play a role in Cer-mediated cell signaling.

i. Chemistry

a. Synthetic Strategy and Selection of 2′-OHCers

The strategy and synthesis for obtaining 2′-OHCers are described below. Scheme 1 shows some exemplary 2′-OHCers.

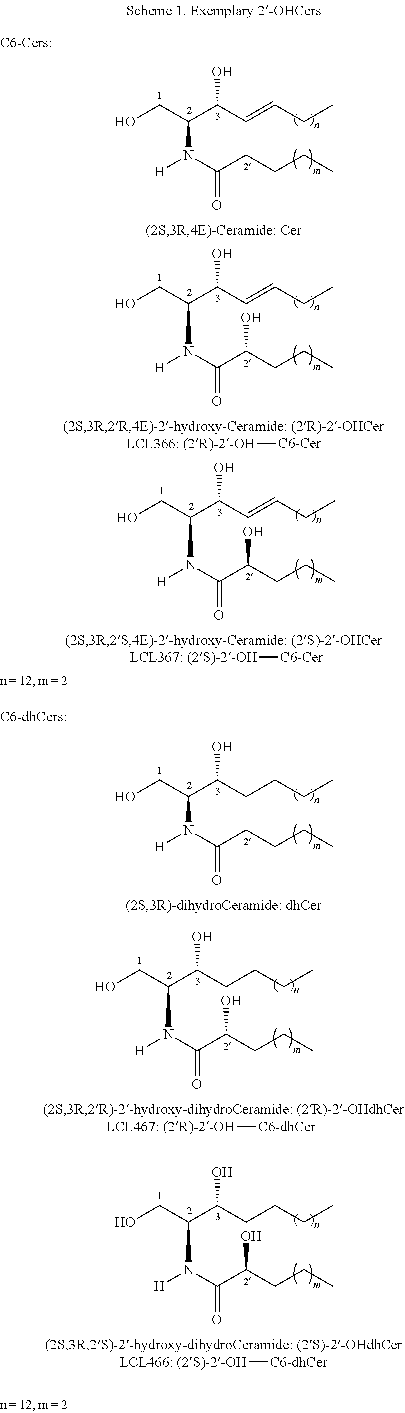

A challenge in the synthesis of naturally occurring 2′-OHCer is to construct the amide bond between the sphingoid base backbone and the appropriate 2-OH fatty acid (FA) moiety, while preserving the stereochemistry of the used building blocks (M. Inagaki, et al., Eur. J. Chem. (1998), p. 129; N. Asai, et al., J. Nat. Prod. 64 (2001), p. 1210; H. Azuma, et al., J. Org. Chem. 68 (2003), p. 2790; P. Radhika, et al., Chem. Pharm. Bull. 52 (2004), p. 1345; H. Azuma, et al., J. Med. Chem. 46 (2003), p. 3445). In all reported cases, beside the necessity to make the appropriate sphingoid base precursor, the synthesis of the R-isomer of any long chain 2-OHFAs was also required, since these lipids are not commercially available. All previous methods suffer from many drawbacks, namely tedious synthesis procedures and multiple purification steps, which make them impractical for a large-scale preparation of varied chains 2′-OHCers. Described herein are synthetic methods of synthesis of 2′-OHCers capable of producing a chromatographically separable set of their two diastereoisomers simultaneously, when racemic mixtures of 2-OHFAs are used in the key synthetic step. The methods were developed based on two observations. First, the introduction of the acetonide protective group into the sphingolipid system facilitated the amide bond formation reaction and secondly, this protecting group was used as a separation handle for polyol isomers and their analogs (H. Azuma, et al., J. Org. Chem. 68 (2003), p. 2790; H. Azuma, et al., J. Med. Chem. 46 (2003), p. 3445; V. N. Odinkov, et al., J. Org. Chem. 39 (2003), p. 952; S. E. Bode, et al., Org. Lett. 4 (2002), p. 619).

Described herein are synthetic methods of making pure isomers of 2′-hydroxy-cers and their corresponding acetonide intermediates as described elsewhere herein. For example, the synthetic methods described herein can produce C4-, C6-, C12-, and C16 homologs of (2S,3R,2′R,4E)-OHCer and their corresponding (2S,3R,2′S,4E)-isomers as well as (2S,3R,2′R)-2′-OH—C6-dhCer and (2S,3R,2′S)-2′-OH—C6-dhCer. The synthetic method was confirmed by comparing the structures of 2′-OHCer and 2′-OHdhCer to the model (2′R)- and (2′S)-2′-OH—C4-Cer stereoisomers, which were prepared first from enantiomerically pure 2-hydroxybutyric acids as well as to the commercially available natural 2′-OH—C18-Cer, isolated from cerebroside.

b. Synthesis of 2′-OHCer Stereoisomers

The synthetic routes of 2′-OHCers 7a-d, 8a-d, 9b and 10b are summarized in Scheme 2. The synthetic methods can first include a condensation reaction. For example, a condensation of commercially available R- and S-isomers of 2-hydroxybutyric acid with the known acetonide of sphingosine (H. Azuma, et al., J. Org. Chem. 68 (2003), p. 2790; H. Azuma, et al., J. Med. Chem. 46 (2003), p. 3445). Using a standard procedure for the amide bond formation, that is, dicyclohexylcarbodiimide (DCC), N-hydroxysuccinimide (NHS), and 1-hydroxybenzotriazole (HOBt) in ethylene glycol dimethyl ether (EGDE) solution, ceramides 3a and 4a were obtained in 82% and 95% yields, respectively. Most importantly, these exemplary diastereomeric 2′-OHCers compounds showed a quite different TLC Rf values (0.36 and 0.64, respectively) and opposite optical rotations ([α]_{D} [symbol] +8.9 and −14.7, respectively). Rf values of 3a and 4a strongly suggested that their mixtures should be easily separated under normal column chromatography conditions. A condensation of the longer chain 2-OHFA racemic mixtures with sphingosine acetonides also follows the same pattern.

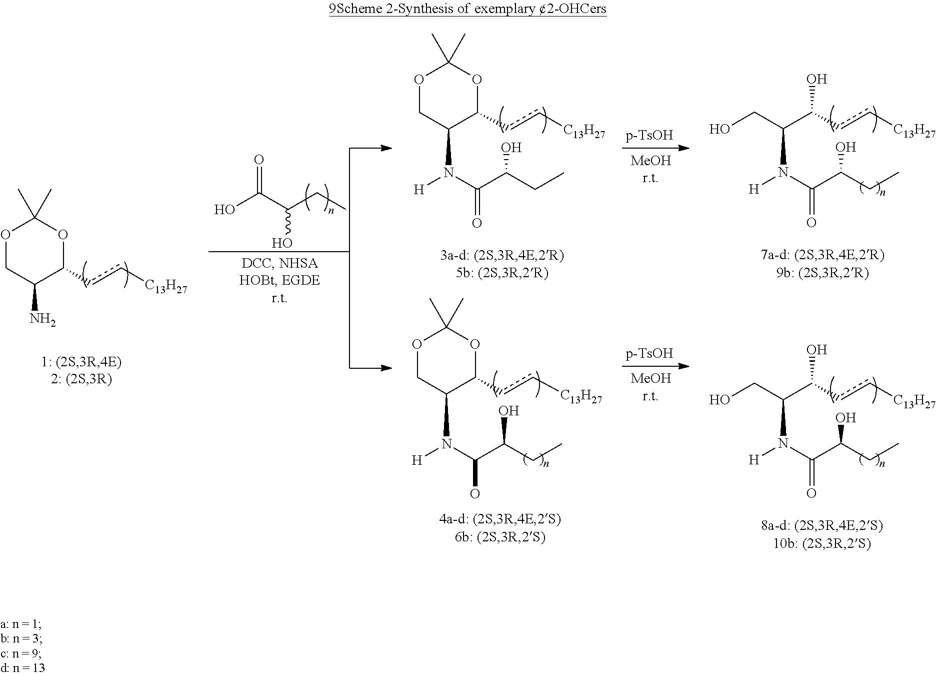

The coupling of the amino component 1 or 2 with the corresponding racemic 2-OHFAs delivered mixtures of (R)- and (S)-isomers of 2′-OHCers (3b-d, 4b-d, 5b, and 6b), these compounds can be separated by normal column chromatography (ΔTLC Rf=0.18-0.2). Final treatment of the separated 2′-OHCer acetonide isomers with p-TsOH in methanol at room temperature provided the desired pure isomers of 7a-d, 8a-d, 9b, and 10b in an overall yield of 26-33%. All synthesized (2′R)—OHCers gave positive values for their optical rotations, which is in full agreement with the reported data for these natural isomers (M. Inagaki, et al., Eur. J. Chem. (1998), p. 129; N. Asai, et al., J. Nat. Prod. 64 (2001), p. 1210; Y. Masuda, et al., Biosci. Biotechnol. Biochem. 66 (2002), p. 1531; H. Azuma, et al., J. Org. Chem. 68 (2003), p. 2790; N. Q. Tien, et al., Arch. Pharm. Res. 27 (2004), p. 1020; P. Radhika, et al., Chem. Pharm. Bull. 52 (2004), p. 1345; Y. Masuda, Eur. J. Org. Chem. (2005), p. 4789; F. Leon, et al., J. Med. Chem. 49 (2006), p. 5830; T. Ishii, et al., J. Nat. Prod. 69 (2006), p. 1080).

c. NMR Characterization of 2′-OHCers

To date, all reported NMR studies of 2′-OHCers and their analogs are focused on the structure determination or confirmation of the isolated and synthesized compounds (M. Inagaki, et al., Eur. J. Chem. (1998), p. 129; N. Asai, et al., J. Nat. Prod. 64 (2001), p. 1210; Y. Masuda, et al., Biosci. Biotechnol. Biochem. 66 (2002), p. 1531; H. Azuma, et al., J. Org. Chem. 68 (2003), p. 2790; N. Q. Tien, et al., Arch. Pharm. Res. 27 (2004), p. 1020; P. Radhika, et al., Chem. Pharm. Bull. 52 (2004), p. 1345; Y. Masuda, Eur. J. Org. Chem. (2005), p. 4789; F. Leon, et al., J. Med. Chem. 49 (2006), p. 5830; T. Ishii, et al., J. Nat. Prod. 69 (2006), p. 1080; M. Kyogasima, et al., J. Biochem. 144 (2008), p. 95; H. Azuma, et al., J. Med. Chem. 46 (2003), p. 3445; J. H. Zhu, et al., Fitoterapia 81 (2010), p. 339). Strategically, most of these experiments focused on the identification of D2O exchangeable doublet at δ 7.20, indicative of the NH proton of the amide part located at the center of Cer structure. Using this proton as a starting point and applying COSY, TOCSY, ROESY, and HSQC experiments, the putative structure of Cer having 2-OHFA moiety was easily confirmed (V. Costantino, et al., J. Med. Chem. 48 (2005), p. 7411; J. H. Jung, et al., J. Nat. Prod. 59 (1996), p. 319). However, there are no reports dealing with the detailed analysis of 2′-OHCer diastereoisomers spectra and their comparison with the ordinary Cers.

The NMR experiments described herein were used to: (i) discriminate between the 2′R and 2′S-isomers of the synthesized short and long chain 2′-OHCers, (ii) compare their spectra to the natural long chain 2′-OHCers and model non-hydroxylated analogs, and (iii) analyze the influence of the additional hydroxy group in the N-acyl part on the structure of the extended polar head.

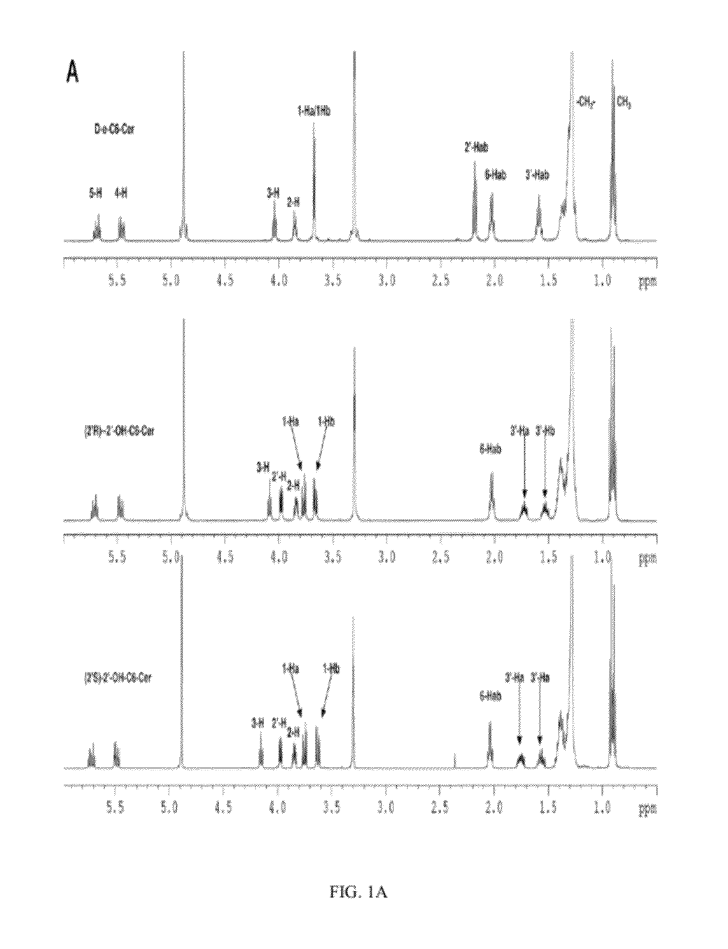

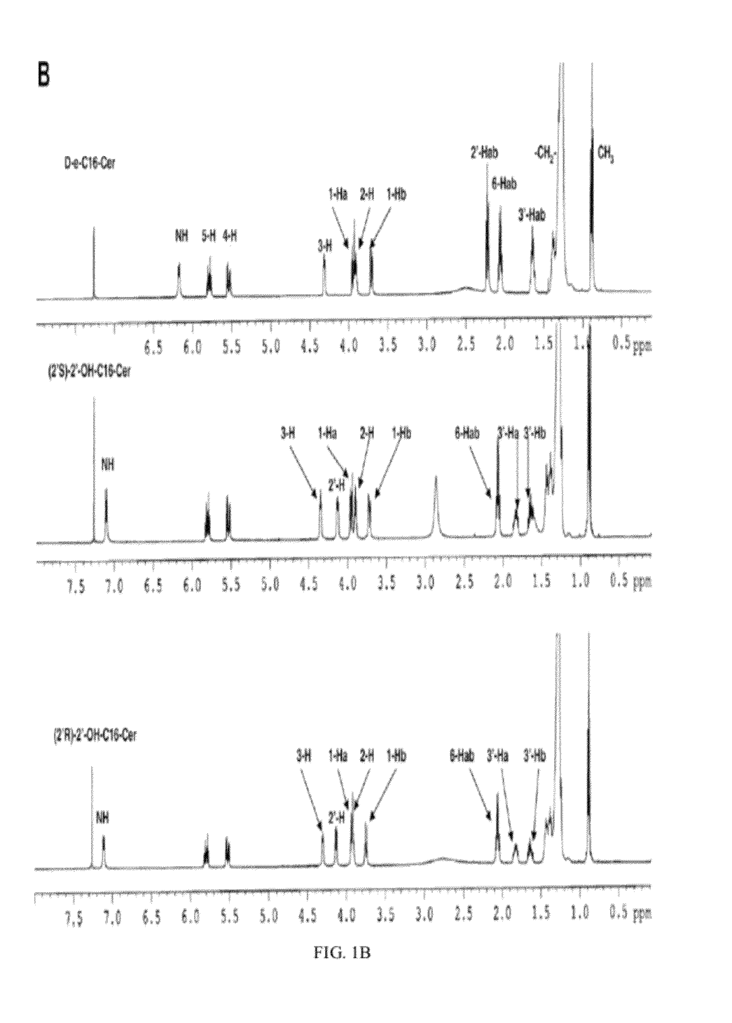

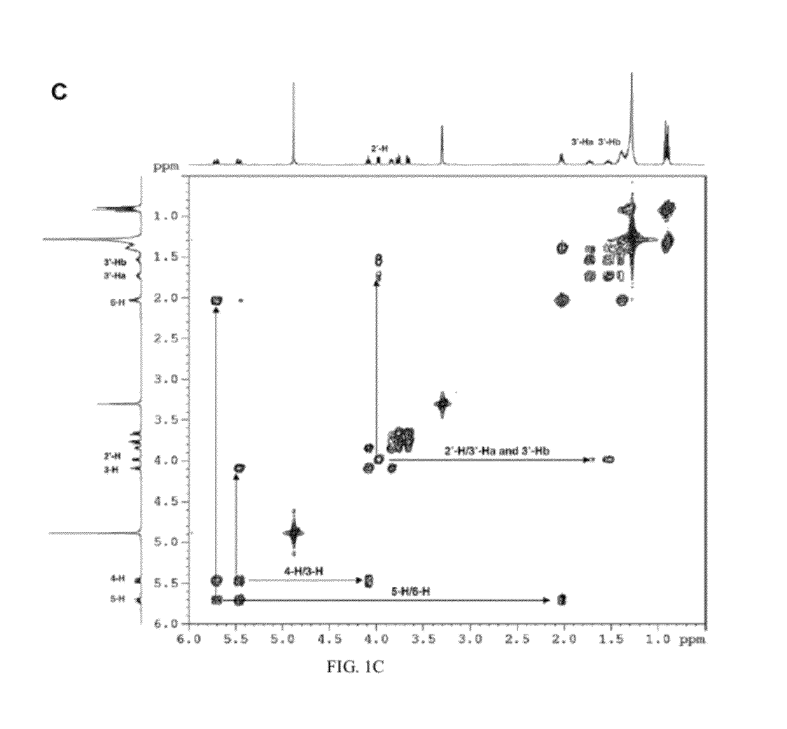

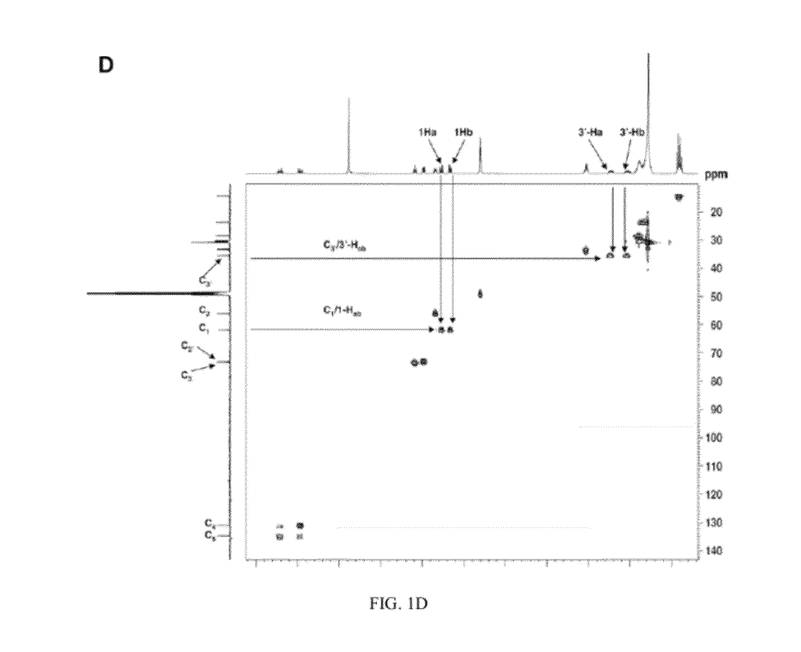

One and two-dimensional ¹H and ¹³C NMR experiments clearly exhibited connectivity, chemical shift values and coupling features expected for 2′-OHCers (FIG. 1A-D and data described herein). Also, they showed significant differences in the values of δH and δC for the polar head protons as well as carbons in comparison to the parent Cers.

Summarizing findings of the study: (i) strong deshielding effects for the 2′-H protons (Δδ2′H=1.72-1.78 ppm, FIGS. 1A and B) and the chiral C2′ carbons (ΔδC2′=35-36 ppm, FIG. 1D) in both isomers were observed, (ii) the NH protons were significantly shifted to the lower field (ΔδNH=0.9 ppm, FIG. 1B) in comparison to the corresponding protons in non-hydroxylated Cers, and (iii) well-separated resonance signals for each methylene 1-Hab and 3′-Hab protons in both 2′-OHCer isomers, in CDCl3 and CD3OD solutions, were present (FIGS. 1A and B). This is a different behavior than usually observed in the spectra of ordinary Cers, where the 1-Ha, 1-Hb and the N-acyl chain methylene proton signals always appeared as one resonance signal (narrow doublet or multiplet) in CD3OD solution (L. Li, et al., Biophys. J. 82 (2002), p. 2067; Z. M. Szulc, et al., Bioorg. Med. Chem. 14 (2006), p. 7083). All of the above spectral features most likely resulted from the extensive intramolecular H-bonding interactions between the hydroxyl groups and the amide group of the polar interface of 2′-OHCer. As a consequence, elevated rigidity of the extended polar heads in 2′-OHCers is expected (L. Li, et al., Biophys. J. 82 (2002), p. 2067).

All NMR spectra of varied chain 2′-OHCer isomers are similar however, the (2′S)-isomers showed higher deshielding effects for their 3-H protons than (2′R)-isomers (Δδ3-H=0.06-0.17 ppm). The observed difference can be the consequence of a proximal position of the 2′-hydroxy amide unit to the 3-H proton in (2′S)-isomer. The juxtaposition of the amide group may potentially cause a deshielding effect of the C═O group on 3-H proton (spatial magnetic anisotropy) (R. J. Abraham, et al., Magn. Reson. Chem. 41 (2003), p. 26). Also, the 3-OH group of 2′-OHCer have increased opportunity to form more H-bonds between the remaining proton-donor and acceptor centers than in non-hydroxylated Cer. Nevertheless, to achieve the full capability of interactions, the polar head of Cer must have its all H-donor/acceptor groups in the gauche spatial disposition (-sc staggered conformer) (L. Li, et al., Biophys. J. 82 (2002), p. 2067). In order to fulfill this spatial arrangement and launch effective H-bonding interactions inside the polar interface, the (2′S)- and (2′R)-isomers may need to twist their N-acyl parts out of the line with the sphingosine backbone. The degree of this distortion could have impact on the overall structure of 2′-OHCers. These structural features can be responsible for the observed distinct deshielding effect for the 3-H protons of 2′-OHCers. Moreover, their NMR resonance signals (δ3H=˜4.30 ppm/(2′S)-isomers versus ˜4.13 ppm/(2′R)-isomers) can be directly used as diagnostics to monitor and confirm the level of optical purity of the prepared 2′-OHCer diastereoisomers.

Based on this exploration, 2′-OHCer can be described as a distinct lipophilic multidentate ligand possessing a set of two rigid three-carbon subunits (C1/C2/C3 and C(O)/C′2/C′3) in the vicinity of its polar interface. This kind of ligand has the capability to control its own shape by utilizing electronic and steric factors: H-bonding interactions to impose conformational restriction on polar head and fix the spatial arrangement of both chains. Also, when its environment will change, the multidentate and rigid polar head of 2′-OHCer may interact efficiently with its target protein or other lipids. The proper assessment of 2′-OHCer diastereoisomers' conformational preferences in solution will need more advanced NMR and molecular modeling studies.

### 2. Example 2

### Inhibitory Effects of 2′-OHCers/dhCers on MCF7 Breast Carcinoma Cells Growth

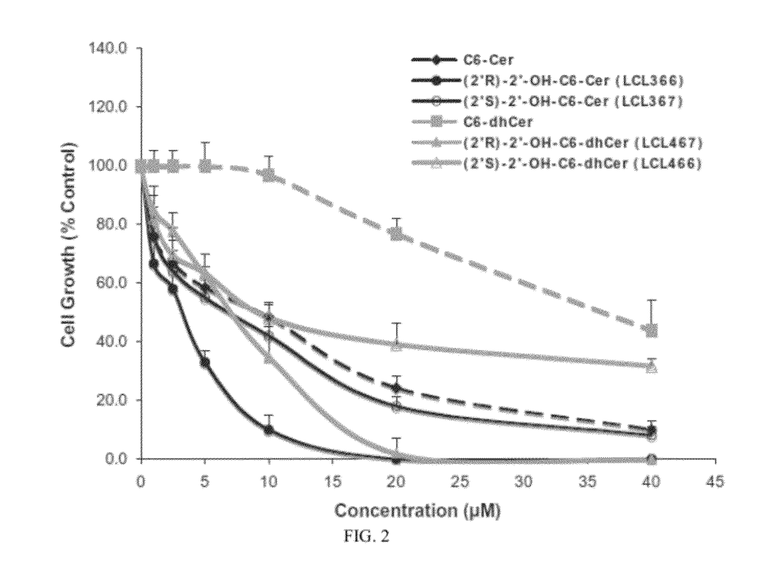

The effects of the newly obtained compounds were examined in MCF7 cells, using 2′-OH—C6-Cer and 2′-OH—C6-dhCer stereoisomers, representing cell permeable short chain homologs of 2′-OHCers/dhCers. Their activity was compared to the effects of d-e-C6-Cer and d-e-C6-dhCer, basic tools commonly used in the sphingolipid research (Z. M. Szulc, et al., Bioorg. Med. Chem. 14 (2006), p. 7083; I. Sultan, et al., Biochem. J. 393 (2006), p. 513; J. V. Chapman, et al., Biochem. Pharmacol. 80 (2010), p. 308). When added to culture media, these analogs showed high antiproliferative activities on cell growth (FIG. 2, 24 h treatments). IC₅₀ values for (2′R)- and (2′S)-isomer of 2′-OH—C6-Cers and 2′-OH—C6-dhCers were established as ⅜ μM and 8/12 μM, respectively. Under identical conditions, the IC50 values for C6-Cer and C6-dhCer were 12 μM and 38 μM, respectively. All synthesized 2′-OH-Cers/dhCer were more potent than the parent compounds and the naturally occurring (2′R)-isomers had significantly stronger antiproliferative effects as compared to the (2′S)-isomers.

The cytotoxic effect of 2′-OH—C4-Cer isomers was also tested. Both isomers showed a very similar inhibitory effects on cell growth with IC₅₀ values corresponding to 7 μM and 9 μM for the (2′R)- and the (2′S)-isomer, respectively. It was previously reported, the (2′S)-2′-OH—C3-Cer was much more potent then its (2′R)-isomer in HL60 leukemia cells (H. Azuma, et al., J. Med. Chem. 46 (2003), p. 3445).

The potent antiproliferative effect of the (2′R)-2′-OH—C6-Cer was also observed with human cervical cancer HeLa cells and human lung adenocarcinoma A549 cells, indicating that the effect is not limited to breast cancer cells only. A similar trend, higher cytotoxic activity of 2′-OHCers isolated from different natural sources in comparison to their non-hydroxylated analogs was previously reported in HL60, K562, A-549, HepG2, Hep3B and BGC-823 cells lines (M. Minamino, et al., Microbiology 149 (2003), p. 2071; M. Kyogasima, et al., J. Biochem. 144 (2008), p. 95; X. Li, et al., Fitoterapia 78 (2007), p. 490).

The data show that the introduction of the hydroxyl group at position 2′ in the N-acyl part of Cer not only maintains but also enhances the activity of 2′-OH—C6-dhCer over its normal and desaturated analogs. These findings are also of interest when considering other studies, showing higher cytotoxicity of phytoCers than the corresponding dhCer and Cer analogs in SK-N-BE(2)C cells (O. Hwang, et al., Mol. Pharmacol. 59 (2001), p. 1249). All these observations extend the existing ‘Cer cytotoxicity paradigm, which states that the trans double bond between C4 and C5 atoms in the Cer backbone is essential for its elevated activity and pro-apoptotic responses (L. M. Obeid, et al., Science 259 (1993), p. 1769; I. Sultan, et al., Biochem. J. 393 (2006), p. 513; A. Bielawska, et al. J. Biol. Chem. 268 (1993), p. 26226; B. Ogretmen, et al., J. Biol. Chem. 277 (2002), p. 12960). Thus, the cytotoxic activity of Cer seems to be rendered by the presence of the double bond in the vicinity of its polar head and/or presence of an additional hydroxyl group at position 2′ or 4. The NMR studies pinpoint these electronic features of the Cer structure as responsible for its polar head enhanced rigidity (B. Ogretmen, et al., J. Biol. Chem. 277 (2002), p. 12960). This can facilitate the molecule's molecular recognition and enhance its binding to the target regulatory proteins (K. Kitatani, et al., Cell. Signalling 20 (2008), p. 1010; E. Bieberich, Future Lipidol. 3 (2008), p. 273). The formation of that lipid-protein complex can be responsible for the observed elevated cytotoxicity of 2′-OHCers and 2′-OHdhCer.

### 3. Example 3

### Cellular Levels of 2′-OHCers/dhCers

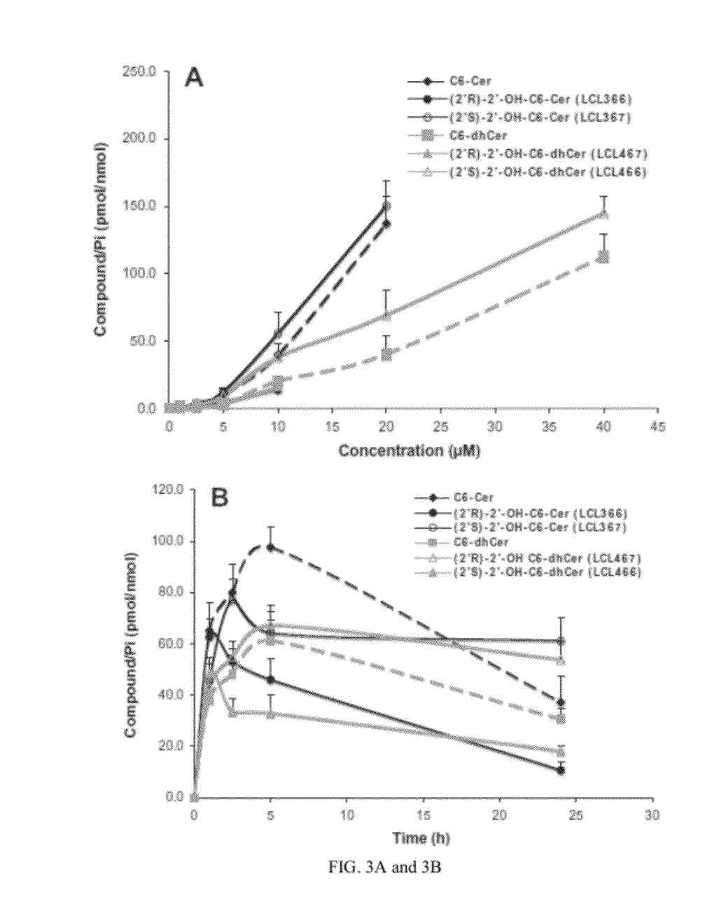

Cellular levels of the representative C6-analogs in MCF7 cells were established by MS analysis. Experimental data from cell treatments at 24 h showed a dose-dependent increase for all tested analogs, however, at different rates (FIG. 3A). (2′S)-Isomer (LCL367) showed the highest level of the C6-Cer series whereas the (2′R)-isomer (LCL366) showed the lowest level and C6-Cer was in the middle. A similar pattern was observed for the C6-dhCer series.

Cellular uptakes for 10 μM treatments were fast and at comparable levels up to ˜1 h, with the highest levels for LCL366 and C6-Cer (FIG. 3B). Following a time course, LCL366 was progressively decreased to the lowest level among all tested analogs at 24 h. LCL367 was elevated up to 2.5 h followed by a small decrease at 5 h and plateau to 24 h. C6-Cer was progressively increased up to 5 h followed by decrease at 24 h. C6-dhCer followed the curve of C6-Cer, however at a lower rate. The 2′S-isomer of 2′-OH—C6-dhCer, LCL466, was elevated up to 5 h reaching the level of C6-dhCer and remaining constant up to 24 h. The (2′R)-isomer of 2′-OH—C6-dhCer, LCL467, showed the lowest level, remaining in a plateau between 2.5-24 h.

Differences in the cellular levels between (2′R)- and (2′S)-isomers for 2′-OHCers and 2′-OHdhCers were observed, with (2′R)-isomers being utilized over time similarly to the parent compounds, whereas (2′S)-isomers being stable.

These results can show different metabolism of these compounds and/or their binding to the specific protein targets.

### 4. Example 4

### Effects of 2′-OHCers/dhCers on Bioactive SPLs in MCF7 Human Breast Carcinoma Cells

Cellular effects of 2′-OHCers on endogenous SPLs and their comparison to the actions of ordinary Cers have not yet been investigated. Using the LC-MS/MS method and monitoring changes in Cer species-Sph-S1P balance we generated a set of preliminary data pinpointing metabolic junctures and differences between the actions of model 2′-OH—C6-Cer diastereoisomers and their parent analogs.

Studies assessing the roles of Cer in signal transduction phenomena and in regulating tumor cell responses to chemotherapy have shown that the active agonists, stress factors or cytotoxic drugs, usually cause an increase of the endogenous Cers, especially C14-, C16-, and C18-Cers, followed later by cell death (M. Mimeault, FEBS Lett. 530 (2002), p. 9; K. Kitatani, et al., Cell. Signalling 20 (2008), p. 1010; B. Ogretmen et al., Nat. Rev. Cancer 4 (2004), p. 604). This response was always observed after cell treatment with exogenous C2- and C6-Cers at the appropriate concentration (L. M. Obeid, et al., Science 259 (1993), p. 1769; A. Bielawska, H. M. et al., J. Biol. Chem. 268 (1993), p. 26226; B. Ogretmen, et al., J. Biol. Chem. 277 (2002), p. 12960; E. Bieberich, Future Lipidol. 3 (2008), p. 273; B. Ogretmen et al., Nat. Rev. Cancer 4 (2004), p. 604 I.-N. Park, et al., Drug Metab. Dispos. 9 (2004), p. 893). C2- and C6-dhCers are commonly used as a negative control for ceramide action (A. Bielawska, H. M. et al., J. Biol. Chem. 268 (1993), p. 26226; C. L. Chik, et al., Mol. Cell. Endicronol. 218 (2004), p. 175). However, it is limited only to C2-dhCer as was previously reported (A. Bielawska, H. M. et al., J. Biol. Chem. 268 (1993), p. 26226; N. D. Ridgeway et al., Biochim. Biophys. Acta 1256 (1995), p. 57). As shown in FIG. 2, C6-dhCer caused an inhibitory effect on cell growth at concentrations higher than 10 μM and was metabolized to C6-Cer and cellular dhCers followed by their desaturation to the cellular Cers (See FIGS. 6-8).

MCF7 control cells showed the following Cer composition: ˜87% Cers, 8% dhCers and ˜5% 2′-OHCers. Among the Cers species C24-, C24:1, and C16-Cers are the major components (˜31%, 44% and 20%, respectively). The remaining C14-, C18-, C18:1-, and C20-Cers represent almost equal values. Among the dhCer species C16-dhCer is the major component (˜60%) followed by C24:1-dhCer (˜12%). The remaining C14-, C20-, and C24-dhCers represent almost equal values. Among the 2′-OHCer species only C24-, C26-, C16-, C24:1-, and C26:1 (˜24%, 22%, 19%, 18%, and 17%, respectively) represent components of this class. Similar delinquent presence and elevated levels of long chain 2′-OHCer species were found in some mammalian tissues and organs (Hama, H., Biochem. Biophys. Acta, 180, (2010), p. 405).

i. Stereospecific effects of 2′-OH—C6-Cers on Cellular 2′-OHCers, Cers, Sph, and SW

a. Concentration-Dependent Effects

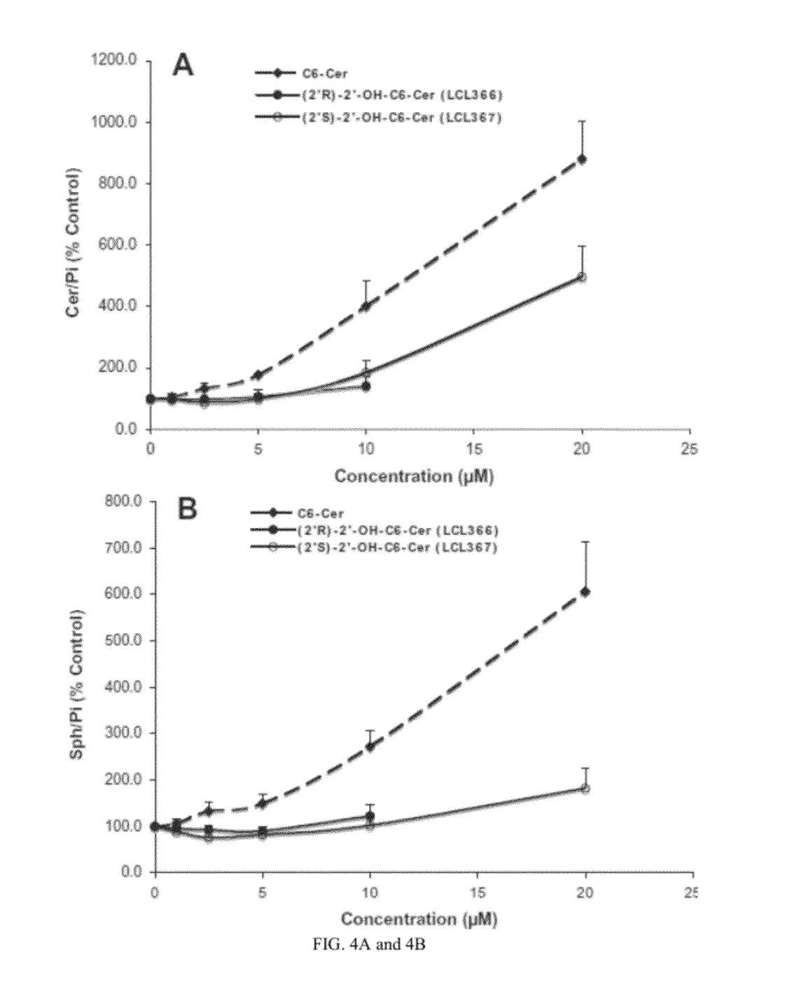

Surprisingly, as observed for 24 hr treatment, both tested isomers of 2′-OH—C6-Cers (2.5-20 μM) showed no effect on endogenous 2′-OHCer levels. For the maximal concentration used, 10 μM LCL366 and 20 μM LCL367, only a small increase was observed (up to 130%; C24- and C24:1-Cers for LCL366; C16-, C24-, and C24:1-Cers for LCL367). The results indicate that 2′-OH—C6-Cers cannot serve as good competitive substrates/inhibitors for enzymes of Cer metabolism or they are not efficiently delivered to the cellular compartment where their active metabolic target can be present. However, it was previously shown that natural 2′-OHCer served as a good substrate for neutral ceramidase in vitro (S. El-Bawab, et al., J. Lipid Res. 43 (2002), p. 141). It was also shown that acidic ceramidase controlled intracellular 2′-OHCer level in vivo (Y. Sun, et al., Hum. Mol. Genet. 16 (2007), p. 957). Interestingly, C6-Cer itself was not converted to the cellular 2′-OHCer analogs but an increase in Sph level was clearly noticed in this case (vide infra, see FIG. 4B). This indicates that the level of 2-OHFA-CoA or FA2H enzyme expression in cancer cells is low. All these observations are in conflict with results showing formation of 2′-OH—C18-Cer from 2-OH—C18-FA-CoA (racemic mixture) and dhSph in HEK293T cells (Y. Mizutani, et al., J. Lipid Res. 49 (2008), p. 2356).

Isomers of 2′-OH—C6-Cers did not elevate cellular Cer as effectively as C6-Cer (FIG. 4A). Also, they did not elevate cellular Sph as was observed for C6-Cer (FIG. 4B). Both, C6-Cer and LCL367 (2′S-isomer), elevated cellular SIP (800% and 600% for 20 μM treatments, respectively), whereas LCL366 (2′R-isomer), had no effect on SIP.

C6-Cer caused a significant dose-dependent increase of endogenous Cer and Sph (FIG. 3A, up to 900% and 600% for 20 μM, respectively). LCL367 followed the pattern of C6-Cer but with a much lower effect (up to 500%/Cer and 150%/Sph for 20 μM). LCL366 only slightly increased endogenous Cer (up to 130%) and had no effect on Sph as noticed for 10 μM treatment (the highest possible concentration used).

The effects of these compounds on endogenous Cer species were also examined. The results showed that C6-Cer elevated all Cer species starting from 5 μM. The highest extent was observed for C18-Cer, C14-, and C16-Cers (˜80-, 40-, and 20-fold, respectively, for 20 μM treatment). Increase in the other Cers was to a much lower extent (C24:1-Cer, fivefold and C24-Cer only ˜0.2-fold). LCL367, the 2′S-isomer, followed the pattern of C6-Cer with a specific increase in C18-Cer (30-fold), C14-Cer (20-fold), C16-Cer (sevenfold), C24:1-Cer (fourfold) and C24-Cer (twofold). LCL366, the 2′R-isomer, up to 5 μM had no effect on Cer species, however at 10 μM a very moderate elevation of C18-Cer (˜5-fold), C14-Cer (threefold), C16-Cer (1.7-fold) and decrease of C24-Cer and C24:1-Cer (to 70% and 50%, respectively) was observed. Again, the (2′R)-isomer behaved differently from (2′S)-isomer and C6-Cer.

b. Time-Dependent Effects

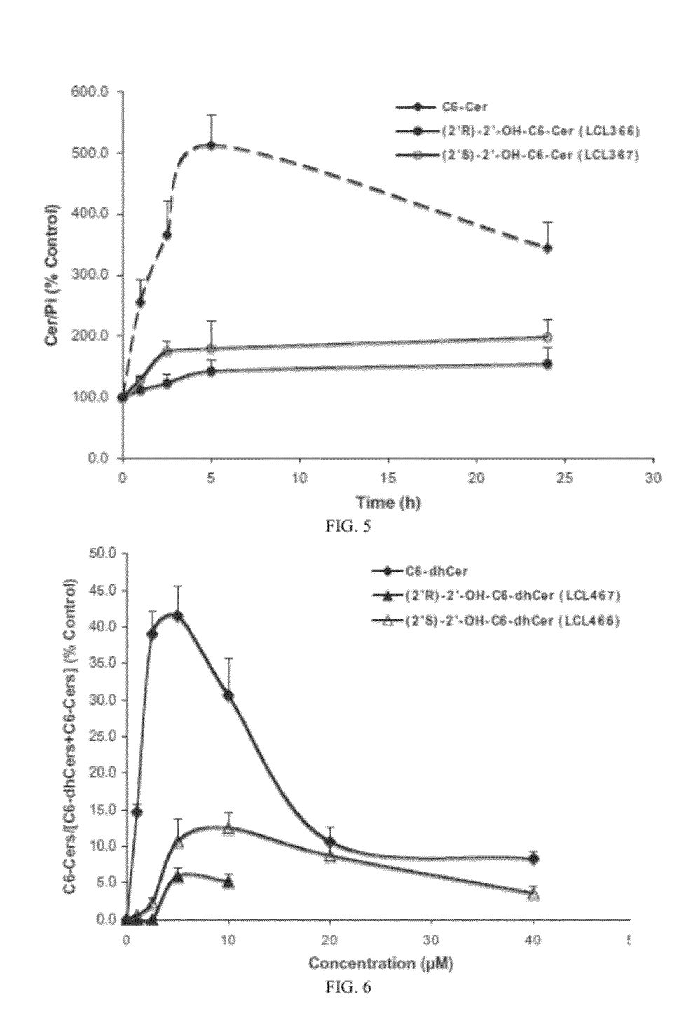

Time-dependent effects on cellular Cers were determined for 10 μM treatments (FIG. 5). Both, LCL366 (2′R) and LCL367 (2′S) were not effective in formation of cellular Cer as compared to C6-Cer. Some elevations (˜140%/LCL366 and ˜180%/LCL367) were observed up to 5 h with a plateau up to 24 h. These effects were different from the action of C6-Cer, where cellular Cers increased rapidly (500%) up to 5 h and decreased (350%) at 24 h. Again, a small increase in cellular Cer by LCL366 did not correlate with its strong antiproliferative effect (IC50/24 h=3 μM, FIG. 2A).

In summary, isomers of 2′-OH—C6-Cers influenced differently bio-transformations of the cellular SPLs as compared to C6-Cer. (2′S)-Isomer showed some similarities to the action of C6-Cer if used at higher concentration and during longer treatment. The observed lack of influence of the (2′R)-isomer on basic cellular SPLs in MCF7 cells and its high cytotoxicity can not be related to its anabolic transformations, which usually favor hydrolysis and generation of Sph and long chain Cers (I. Sultan, et al., Biochem. J. 393 (2006), p. 513; J. V. Chapman, et al., Biochem. Pharmacol. 80 (2010), p. 308; A. Bielawska, et al., J. Biol. Chem. 268 (1993), p. 26226; B. Ogretmen, et al., J. Biol. Chem. 277 (2002), p. 12960). Results indicate that this isomer or its unidentified yet metabolites directly target some key regulatory enzymes (e.g., phosphatases, kinases or caspases) (A. Carpinteiro, et al., Cancer Lett. 264 (2008), p. 1; K. Kitatani, et al., Cell. Signalling 20 (2008), p. 1010; A. Mukhopadhyay, et al., FASEB J. 23 (2009), p. 751. Our preliminary data support this hypothesis, since some (2′R)—OHCers had a higher potency than non-hydroxylated-Cer for the PPA2 enzyme activation (P. Roddy, D. Perry, and Y. A. Hannun, unpublished data).

An uncommon profile of biological action for the (2′R)-isomer, representing natural 2′-OHCers, was discovered by comparison to the effects of C6-Cer and its unnatural (2′S)-isomer.

ii. Stereospecific Effects of 2′-OH—C6-dhCers on Cellular SPLs

Since a metabolic conversion of C6-dhCer to C6-Cer was observed for 24 h treatment (FIG. 6), conversions of 2′-OH—C6-dhCer diastereoisomers was also investigated, LCL466 (2′S-isomer) and LCL467 (2′R-isomer) to their desaturated counterparts as well as effects on endogenous dhCers, Cers and both sphingoid bases.

In general, 2′-OH—C6-dhCers did not serve as a good substrate for dhCer desaturase since only a small percent of conversion (up to 12%) of LCL466 to the corresponding LCL367 and LCL467 to the corresponding LCL366 (up to 5%) was observed as compared to 42% conversion for C6-dhCer.

a. Concentration-Dependent Effects of 2′OH-dhCers on Endogenous SPLs

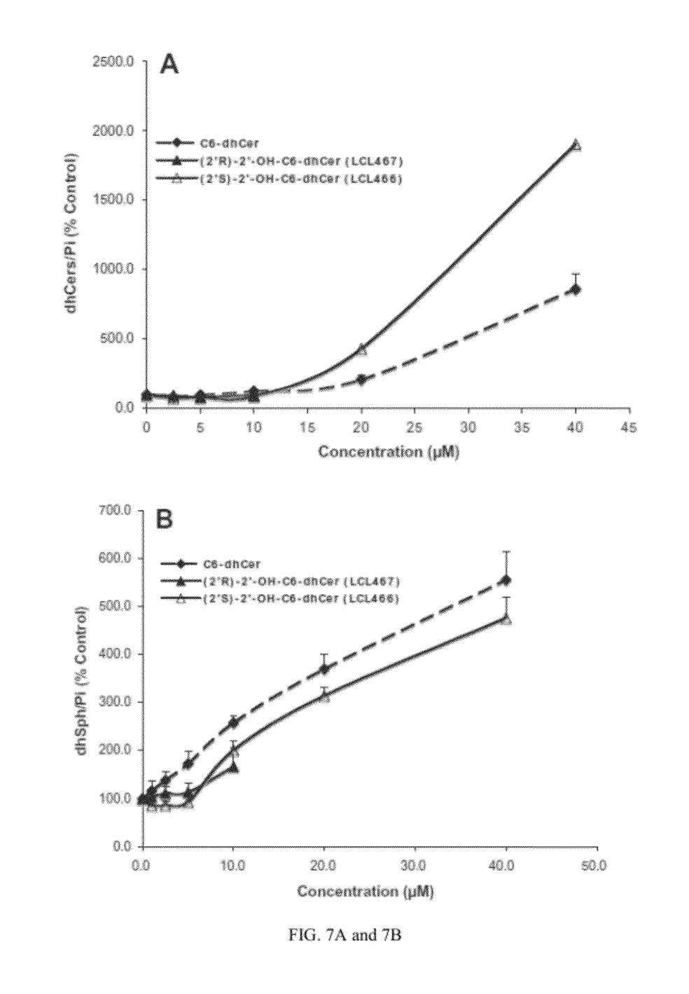

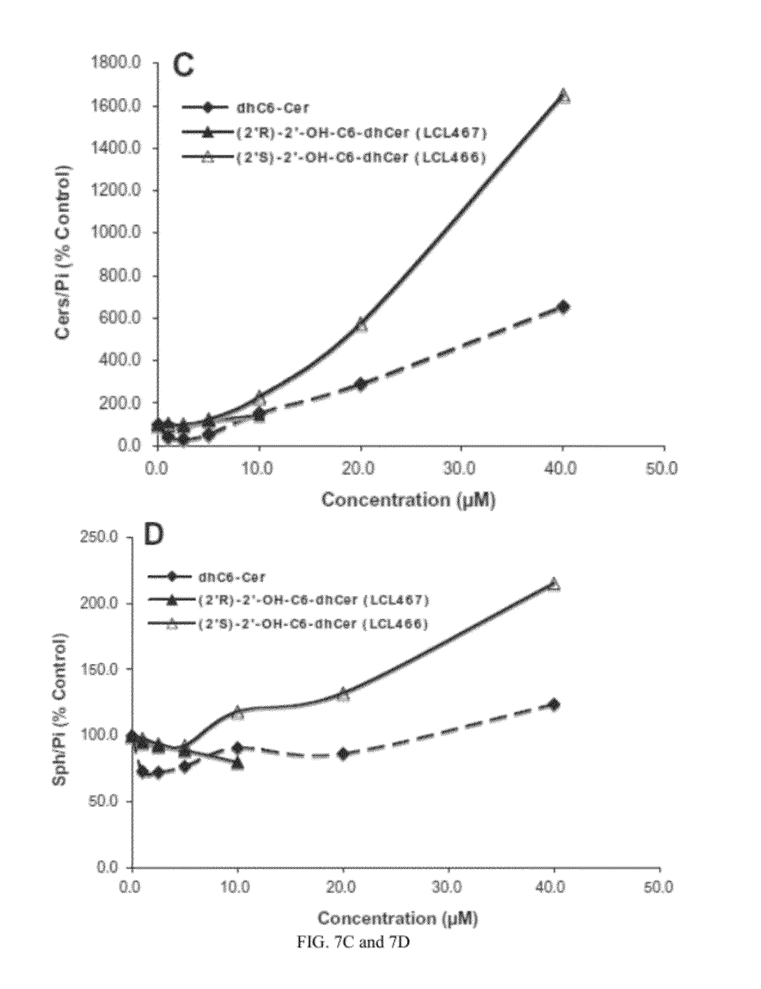

(A) Effect on Endogenous dhCers (FIG. 7A)

Up to 10 μM of C6-dhCer and both isomers of 2′-OH—C6-dh-Cer did not induce elevation of cellular dhCers. This concentration represented the highest concentration tested for (2′R)-isomer, since 20 μM treatment caused total cell death as shown in FIG. 2. LCL466, (2′S)-isomer, elevated cellular dhCer to the highest extent (˜19-fold/40 μM treatment). In comparison, C6-dhCer increased cellular dhCer only ˜7-fold/40 μM treatment. Regarding dhCer species (results not shown), both LCL466 and C6-dhCer elevated mostly dhC16-Cer, the major dhCer for MCF7 control cells (up to 25- and 13-fold, respectively). LCL467 did not elevate C16-dhCer. Interestingly, it slightly decreased the other dhCers, especially C14-dhCer (to 25% control).

(B) Effect on Endogenous Cers (FIG. 7B)

Effect of these analogs on endogenous Cer was different from their effects on dhCers (FIG. 7A). The highest elevation was observed for C6-dhCer for the full range of the concentration used, reaching 550% for 40 μM. LCL 466, the (2′S)-isomer, starting from 5 μM followed the effect of C6-dhCer, however, to a lower extent (˜150% for 40 μM treatment). LCL467 as similarly shown for cellular dhCer, did not affect cellular Cer up to 5 μM, however a 10 μM treatment increased Cer to ˜150%. Regarding Cer species, all compounds elevated mostly C18- and C14-Cers, followed by C24:1-Cer, however at different rates (results not shown). C16-Cer was elevated up to ˜50 fold by 40 μM LCL466 and C6-dhCer, whereas 1-5 μM LCL467, caused a rather similar decrease (up to 60%) of this Cer.

(C) Effect on Sphingoid Bases

MCF7 control cells (1×106) contain ˜20 pmols Sph and only ˜1 pmol of dhSph. Dose-dependent increase of cellular dhSph was caused by LCL466, (2′S)-isomer, increasing ˜16-fold for 40 μM treatments, whereas LCL467 did not affect cellular dhSph. C6-dhCer had small effect on dhSph elevation (˜5-fold/40 μM) (FIG. 7C). LCL466 also caused elevation of cellular Sph (FIG. 7D), however, to much lower extends in comparison to dhSph. LCL467 showed the same small decrease of Sph (to ˜80%), whereas C6-dhCer decreased Sph at a low concentration (to ˜70%), showing some recovery at a higher concentration.

(D) Time-Dependent Effects

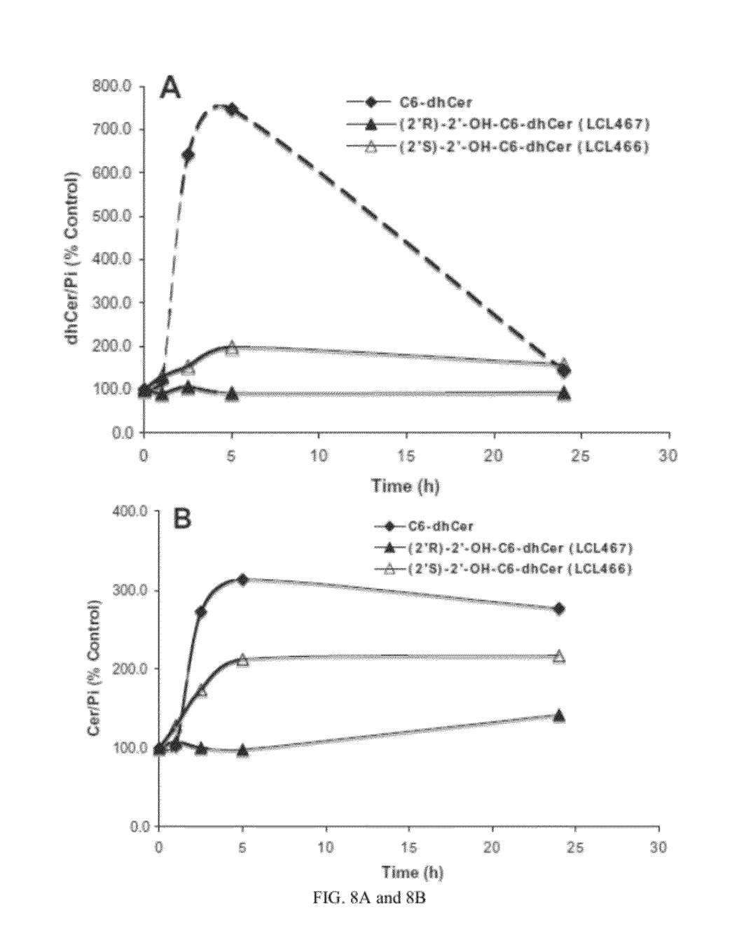

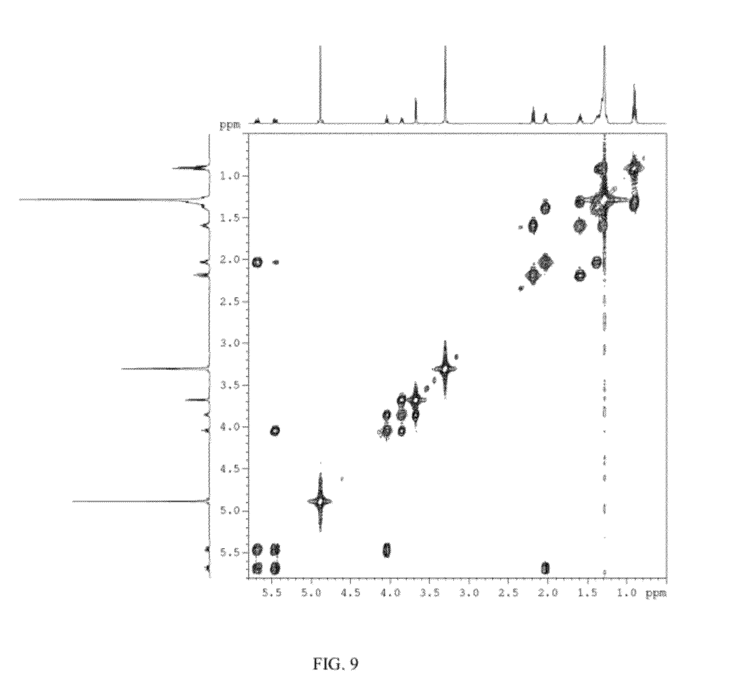

Time-dependent elevation of cellular dhCer (FIG. 8A) and Cer (FIG. 8B) were observed for 10 μM LCL466, up to 5 h, reaching 200% for both lipids. 10 μM C6-dhCer elevated cellular dhCers to 750% and Cers to 310%. LCL467, the (2′R)-isomer, did not affect cellular dhCers nor Cers for this period of time; however, at 24 h Cer was slightly elevated (˜140%) whereas dhCer remained unchanged. At 24 h, dhCer generated by C6-dhCer decreased to almost control level whereas cellular Cer was only slightly decreased (from 310% to 280%). Regarding effect of LCL466 at 24 h, both cellular dhCer and Cer were the same as for 5 h treatment. Among the dhCer species, LCL466 and C6-dhCer elevated all dhCer species, whereas LCL467 decreased long chain dhCers (˜10-25%) and increased very long chain dhCers (˜20-30%), data not shown. LCL466 and C6-dhCer elevated all Cer species, particularly C18-, C14-, and C16-Cers however to very different extent. Cellular dhSph followed the curves of dhCer whereas Sph was decreased by both 2′-OH-dhCers and slightly increased by C6-dhCer.

2′-OH—C6-dhCer diastereoisomers showed different effects on cellular dhCers, Cers, dhSph, and Sph. The (2′R)-isomer was not effective, whereas (2′S)-isomer acted as a very potent inducer of these lipids being three time more effective than C6-dhCer. The results indicate that LCL466 can undergo anabolic transformations and/or may regulate the action of dhCer metabolizing enzymes. DhCer/Cer synthases or dhCer desaturase can be considered as its targets. Most likely, LCL466 acts as an inhibitor of dhCer synthase or activator of dhCer ceramidase, since elevation of dhSph was observed. Moreover, LCL466 can additionally act as an inhibitor of dhCer desaturase, since cellular dhCers generated by this compound were not so efficiently transformed to their oxidized analogs as those generated by the ordinary C6-dhCer (50/25 and 50/13-fold ratio for C16-Cer/C16-dhCer, respectively).

### 5. Example 5

### Chemistry

All solvents, general reagents and short chain (2R)- and (2S)-4-hydroxybutyric acids were purchased from Aldrich-Sigma & Fluka. d-e-Sph and d-e-dhSph were purchased from Avanti. Long chain 2-hydroxy-FAs and a model sample of naturally occurring (2′R)-2′-hydroxy-C18-ceramide were purchased from Matreya. The homogeneity and optical purity of these lipids were checked by NMR, MS spectrometry and optical rotation.

### i. Natural (2′R)-2′-hydroxy-C18-ceramide (from Matreya, cat. #1323)

TLC Rf=0.40-42 (CHCl₃—CH₃OH, 45:5, v/v); 1H NMR (500 MHz, CDCl₃, δ in ppm): 7.08 (d, 1H, J=8.2, NH), 5.79 (dtd, 1H, J=15.1, 6.8, 1.0, 5-H), 5.52 (ddt, 1H, J=15.1, 6.8, 1.1, 4-H), 5.34 (m, ˜0.5H, C′H═C′H), 4.30 (t, 1H, J=6.0, 3-H), 4.13 (m, 1H, 2′-HR), 3.92 (m, 2H, 1-Ha and 2-H), 3.74 (m, 1H, 1-Hb), 2.0 (m, ˜3H, C(6)H2 and C′H2), 1.82 (m, 1H, 3′-Ha), 1.64 (m, 1H, 3′Hb), 1.26 (m, ˜48H, CH2), 0.88 (t, 6H, J=7.1, CH3); 13C NMR (CDCl₃) δ 174.4 (C═O), 134.3 (C5), 130.0 (C′H═C′H), 128.4 (C4), 74.3 (C3), 72.48 (C′2), 62.2 (C1), 54.5 (C2), 34.8 (C′3), 32.3 (C6), 31.9, 29.7, 29.6, 29.5, 29.4, 29.3, 29.2, 29.1, 27.2, 25.0 and 22.7 (C7-C17 and C′4-C′15), 14.0 (C′H3 and CH3). ESI-MS (CH3OH, relative intensity, %) m/z 664.6 ([M1H]+, 32), 628.6 ([M1H—H2O]+, 4), (582.6 ([M2H]+, 40), 564.2 ([M2H—H2O]+, 8), 546.7 (([M2H-2H2O]+, 4). Calcd for C₄₂H₈₂NO₄ m/z 663.62 [M1] and C₃₆H₇₁NO₄ m/z 581.54 [M2].

1,3-Isopropylidene-(2S,3R,4E)-sphingosine (1) and 1,3-isopropylidene-(2S,3R)-dihydrosphingosine (2) were prepared from the corresponding sphingoid bases by a slightly modified procedure described previously (Azuma, H. et al., J. Org. Chem. 68, (2003), p. 2790). Purity of the synthesized compounds was confirmed by NMR spectroscopy and the measurement of their optical rotation values. Reaction progress was monitored by the analytical normal and reverse phase thin layer chromatography (NP TLC or RP TLC) using aluminum sheets with 0.25 mm Silica Gel 60-F254 (Merck) and 0.150 mm C18-silica gel (Sorbent Technologies). Detection was done by the PMA reagent (ammonium heptamolybdate tetrahydrate cerium sulfate, 5:2, g/g) in 125 mL of 10% H2SO4) and the Dragendorff reagent (Fluka) following heating of the TLC plates at 170° C. or by the UV (254 nm). Flash chromatography was performed using EM Silica Gel 60 (230-400 mesh) with the indicated eluent systems. Melting points were determined in open capillaries on Electrothermal IA 9200 melting point apparatus and are reported uncorrected. Optical rotation data were acquired using a Jasco P-1010 polarimeter. ¹H and ¹³C NMR spectra were recorded on Bruker AVANCE 500 MHz spectrometer equipped with Oxford Narrow Bore Magnet. Chemical shifts are reported in ppm on the d scale from the internal standard of residual chloroform (7.26 ppm). Mass spectral data were recorded in a positive ion electrospray ionization (ESI) mode on Thermo Finnigan TSQ 7000 triple quadrupole mass spectrometer. Samples were infused in methanol solution with an ESI voltage of 4.5 kV and capillary temperature of 200° C. Specific structural features were established by fragmentation pattern upon electrospray (ESI/MS/MS) conditions, specific for each of the compounds studied (J. Bielawski, et al., Methods 39 (2006), p. 82; J. Bielawski, et al., Methods Mol. Biol. 579 (2009), p. 443).

### ii. Preparation of 1,3-isopropylidene-sphingoid bases

### a. 1,3-Isopropylidene-(2S,3R,4E)-sphingosine (1)

A mixture of (2S,3R,4E)-sphingosine (300 mg, 1.0 mmol), pyridinium p-tolulenesulfonate (98%, 252 mg, 1.0 mmol) and 2,2-dimethoxypropane (6 mL) in dry toluene (10 mL) was stirred at reflux for 4 h. The reaction mixture was then cooled to room temperature and diluted with ethyl acetate (10 mL), washed with saturated sodium bicarbonate solution and water. The collected organic layer was dried over anhydrous magnesium sulfate and filtered. The solvent was evaporated under reduced pressure and the obtained residue was purified by flash column chromatography (CHCl3-CH3OH—NH4OH concd, 15:1:0.025 v/v/v) to give pure 1 (312 mg, 92%) as a pale yellow oil. This material formed a waxy solid upon storage in refrigerator (+4° C.) overnight. TLC Rf=0.43 (CHCl₃—CH₃OH—NH₄OH concd, 15:1:0.025, v/v/v); [α]₀²⁵+11.0 (c 1, CHCl3); [α]₆₅²⁵+38.7 (c 1, CHCl3); 1H NMR (500 MHz, CDCl3) δ 5.82 (dtd, 1H, J=15.2, 7.4 and 1.0, 5-H), 5.37 (ddt, 1H, J=15.1, 7.6 and 1.4, 4-H), 3.85 (m, 2H, 1-Ha and 3-H), 3.54 (dd, 1H, J=10.8 and 11.2, 1-Hb), 2.70 (m, 1H, 2-H), 2.01 (m, 2H, C(6)H2), 1.49 (s, 3H, CH3), 1.42 (s, 3H, CH3), 1.35 (m, 2H, CH2), 1.24 (m, 22H, CH2), 0.88 (t, 3H, J=7.4, CH3), 0.87 (t, 3H, J=7.1, CH3). 13C NMR (500 MHz, CDCl3) δ 137.3, 127.8, 127.7, 100.4, 98.7, 78.4, 65.6, 49.3, 32.6, 32.1, 29.9, 29.8, 29.7, 29.6, 29.5, 29.2, 22.9, 19.8, 19.4, 14.9, 14.4. MS (CH3OH) m/z 340.8 ([MH]+, 100), 282.3 ([MH]+—C3H6O, 15); Calcd for C₂₁H₄₁NO₂ m/z 339.3 [M].

Effect of these analogs on endogenous Cer was different from their effects on dhCers (See FIG. 7A). The highest elevation was observed for C6-dhCer for the full range of the concentration used, reaching 550% for 40 μM. LCL 466, the (2′S)-isomer, starting from 5 μM followed the effect of C6-dhCer, however, to a lower extent (˜450% for 40 μM treatment). LCL467 as similarly shown for cellular dhCer, did not affect cellular Cer up to 5 μM, however a 10 μM treatment increased Cer to ˜150%. Regarding Cer species, all compounds elevated mostly C18- and C14-Cers, followed by C24:1-Cer, however at different rates. C16-Cer was elevated up to ˜50 fold by 40 μM LCL466 and C6-dhCer, whereas 1-5 μM LCL467, caused a rather similar decrease (up to 60%) of this Cer.

### b. 1,3-Isopropylidene-(2S,3R)-dihydrosphingosine (2)

The same procedure, as described for the synthesis of 1, was performed using (2S,3R)-dihydrosphingosine as a starting material. The crude product was purified by flash chromatography (CHCl3-CH3OH, 25:2 v/v) to give pure 2 in 99% yield as a pale yellow oil. This material solidified upon storage in refrigerator (+4° C.) overnight. TLC Rf=0.58 (CHCl3-CH3OH, 25:2 v/v); [α]_{D}²³+31.8 (c 1, CHCl3); [α] [symbol] ²³+97.0 (c 0.5, CHCl3). Remaining spectral and analytical data are identical to the literature references (Azuma, H. et al., J. Org. Chem. 68, (2003), p. 2790).

### iii. General procedure for the preparation of 1,3-isopropylidene-2′-OH-Cers (3a-d, 4a-d) and 1,3-isopropylidene-2′-OH—C6-dihydroCers (5b, 6b)

A well-stirred mixture of 1,3-ispropylidene-(2S,3R,4E)-sphingosine (1, 105 mg, 0.31 mmol) or 1,3-ispropylidene-(2S,3R)-dihydrosphingosine (2, 106 mg, 0.31 mmol), 2-hydroxy fatty acid (0.36 mmol), DCC (99%, 75 mg, 0.36 mmol), NHS (97%, 42 mg, 0.36 mmol) and HOBt (˜5% water, 49 mg, 0.36 mmol) in dry EGDE (5 mL) was stirred at room temperature for 4 h. The reaction mixture was evaporated to dryness under reduced pressure and the obtained residue was purified by flash column chromatography using the suitable solvent system.

### a. (2S,3R,2′R,4E)-1,3-Isopropylidene-2′-hydroxy-C4-ceramide (3a)

Prepared from 1 and (2R)-2-hydroxybutanoic acid in 82% yield after purification by column chromatography (CHCl3-CH3OH, 25:2, v/v). Analytical sample of 3a was prepared by crystallization from ethyl acetate-n-hexane (3:1, v/v) as a white microcrystalline powder, mp 75-77° C. TLC Rf=0.36 (CHCl3-CH3OH, 25:2, v/v); [α]_{D}²²+8.9 (c 1, CHCl₃); [α] [symbol] ²²+19.2 (c 1, CHCl₃); 1H NMR (500 MHz, CDCl₃) δ 6.31 (d, 1H, J=8.6, NH), 5.74 (dtd, 1H, J=15.4, 7.6 and 1.0, 5-H), 5.42 (ddt, 1H, J=15.4, 7.6 and 1.4, 4-H), 4.07 (m, 2H, 2′-HR and 3-H), 3.97 (dd, 1H, J=5.3 and 11.2, 1-Ha), 3.86 (m, 1H, 2-H), 3.64 (dd, 1H, J=9.5 and 11.2, 1-Hb), 2.01 (m, 2H, C(6)H2), 1.82 (m, 3′-Ha, 1H), 1.65 (m, 3′-Hb, 1H), 1.49 (s, 3H, CH3), 1.42 (s, 3H, CH3), 1.24 (m, 22H, CH2), 0.93 (t, 3H, J=7.4, CH3), 0.87 (t, 3H, J=7.1, CH3). MS (CH₃OH) m/z 873.0 ([2M+Na]+, 23), 449.0 ([M+Na]+, 100), 368.0 ([MH—(CH₃)₂CO]+, 5). Calcd for C₂₅H₄₇NO₄ m/z 425.35 [M].

### b. (2S,3R,2′S,4E)-1,3-Isopropylidene-2′-hydroxy-C4-ceramide (4a)

Prepared from 1 and (2S)-2-hydroxybutanoic acid in 95% yield after purification by column chromatography (CHCl₃—CH₃OH, 25:2, v/v). Analytical sample of 4a was prepared by crystallization from ethyl acetate-n-hexane (3:1, v/v) as a white microcrystalline powder, mp 62-63° C. TLC Rf=0.64 (CHCl3-CH3OH, 25:2, v/v); [α]_{D}²²−14.7 (c 1, CHCl3); [α] [symbol] ²²−62.3 (c 1, CHCl3); 1H NMR (500 MHz, CDCl3) δ 6.25 (d, 1H, J=8.2, NH), 5.79 (dtd, 1H, J=15.4, 7.6 and 1.0, 5-H), 5.44 (ddt, 1H, J=15.4, 7.6 and 1.4, 4-H), 4.16 (dd, 1H, J=7.8 and 9.4, 3-H), 4.06 (dd, 1H, J=4.8 and 7.3, 2′-HS), 4.02 (dd, 1H, J=5.2 and 11.2, 1-Ha), 3.84 (m, 1H, 2-H), 3.70 (dd, 1H, J=9.6 and 11.2, 1-Hb), 2.05 (m, 2H, C(6)H2), 1.87 (m, 3′-Ha, 114), 1.68 (m, 3′-Hb, 1H), 1.53 (s, 3H, CH3), 1.46 (s, 3H, CH3), 1.24 (m, 22H, CH2), 0.98 (t, 3H, J=7.4, CH3), 0.90 (t, 3H, J=7.1, CH3); MS (CH₃OH) m/z 873.0 ([2M+Na]+, 60), 870.0 ([2M-2H+Na]+, 100), 449.0 ([M+Na]+, 5), 368.0 ([MH—(CH₃)₂CO]+, 23). Calcd for C₂₅H₄₇NO₄ m/z 425.35 [M].

### c. (2S,3R,2′R,4E)-1,3-isopropylidene-2′-hydroxy-C6-ceramide (3b)

Prepared from 1 and dl-2-hydroxyhexanoic acid in 41% yield after purification by column chromatography (ethyl acetate-n-hexane, 2:1, v/v). Analytical sample of 3b was prepared by crystallization from ethyl acetate-n-hexane (1:3, v/v) as a white microcrystalline powder, mp 77-79° C. TLC Rf=0.22 (ethyl acetate-n-hexane, 1:1, v/v); [α]₀ [symbol] +5.3 (c 0.5, CHCl3); [α] [symbol] [symbol] +16.8 (c 0.5, CHCl3); 1H NMR (500 MHz, CDCl₃) δ 6.36 (d, 1H, J=8.8, NH), 5.77 (dtd, 1H, J=15.1, 7.4, 1.0, 5-H), 5.42 (ddt, 1H, J=15.1, 7.5, 1.4, 4-H), 4.09 (m, 2H, 2′-HR and 3-H), 3.97 (dd, 1H, J=5.6 and 11.2, 1-Ha), 3.88 (m, 1H, 2-H), 3.66 (dd, 1H, J=9.6 and 11.2, 1-Hb), 2.38 (d, 1H, J=4.8, 2′-OH), 2.04 (m, 2H, C(6)Ha), 1.87 (m, 2H, CH2), 1.81 (m, 1H, 3′-Ha), 1.70 (m, 2H, CH2), 1.60 (m, 1H, 3′-Hb), 1.50 (s, 3H, CH3), 1.43 (s, 3H, CH3), 1.24 (m, 32H, CH2), 1.15 (m, 2H, CH2), 0.88 (m, 6H, 2×CH3). MS (CH₃OH) m/z 929.7 ([2M+Na]+, 2), 449.5 (100), 414.4 (([MH—(CH3)2C], 6) 396.2 ([MH—(CH₃)₂CO]+, 48). Calcd for C₂₇H₅₁NO₄ m/z 453.38 [M].

### d. (2S,3R,2′S,4E)-1,3-Isopropylidene-2′-hydroxy-C6-ceramide (4b)

Prepared from 1 and dl-2-hydroxyhexanoic acid in 40% yield after purification by column chromatography (ethyl acetate-n-hexane, 2:1, v/v). Analytical sample of 4b was prepared by crystallization from ethyl acetate-n-hexane (1:3, v/v) as a white microcrystalline powder, mp 68-69° C. TLC Rf=0.41 (ethyl acetate-n-hexane, 1:1, v/v); [α]_{D}²²−11.4 (c 0.5, CHCl3); [α] [symbol] _{S}²²−43.4 (c 0.5, CHCl3); 1H NMR (400 MHz, CDCl3) δ 6.24 (d, 1H, J=8.4, NH), 5.76 (dtd, 1H, J=15.1, 7.4, 1.0, 5-H), 5.42 (ddt, 1H, J=15.1, 7.5, 1.4, 4-H), 4.15 (dd, 1H, J=8 and 9.6, 3-H), 4.06 (q, 1H, J=4.0, 2′-HS), 4.0 (dd, 1H, J=5.6 and 11.2, 1-Ha), 3.81 (m, 1H, 2-H), 3.66 (dd, 1H, J=9.6 and 11.2, 1-Hb), 2.26 (d, 1H, J=4.8, 2′-OH), 2.02 (m, 2H, C(6)Ha), 1.80 (m, 1H, 3′-Ha), 1.58 (m, 1H, 3′-Hb), 1.52 (s, 3H, CH3), 1.43 (s, 3H, CH3), 1.24 (m, 32H, CH2), 1.15 (m, 2H, CH2), 0.85 (m, 6H, 2×CH3). MS (CH3OH) m/z 929.7 ([2M+Na]+, 2), 449.5 (100), 414.4 ([MH—(CH3)2C], 6) 396.2 ([MH—(CH₃)₂CO]+, 48). Calcd for C₂₇H₅₁NO₄ m/z 453.38 [M].

### e. (2S,3R,2′R,4E)-1,3-Isopropylidene-2′-hydroxy-C12-ceramide (3c)

Prepared from 1 and dl-2-hydroxydodecanoic acid in 33% yield after purification by column chromatography (ethyl acetate-n-hexane, 2:3, v/v). Analytical sample of 3c was prepared by crystallization from ethyl acetate-n-hexane (3:1, v/v) as a white microcrystalline powder, mp 85-87° C. TLC Rf=0.22 (ethyl acetate-n-hexane, 2:3, v/v); [α]_{D}²³+5.60 (c 0.5, CHCl3); [α] [symbol] ²³+11.0 (c 0.5, CHCl₃); 1H NMR (500 MHz, CDCl₃) δ 6.32 (d, 1H, J=8.8, NH), 5.74 (dtd, 1H, J=15.1, 7.4, 1.0, 5-H), 5.40 (ddt, 1H, J=15.1, 7.5, 1.4, 4-H), 4.04 (m, 2H, 2′-HR and 3-H), 3.96 (dd, 1H, J=5.2 and 11.2, 1-Ha), 3.86 (m, 1H, 2-H), 3.64 (dd, 1H, J=9.6 and 11.2, 1-Hb), 2.24 (d, 1H, J=4.4, 2′-OH), 2.01 (m, 2H, C(6)Ha), 1.92 (m, 2H, CH2), 1.78 (m, 1H, 3′-Ha), 1.64 (m, 2H, CH2), 1.59 (m, 1H, 3′-Hb), 1.48 (s, 3H, CH3), 1.41 (s, 3H, CH3), 1.24 (m, 32H, CH2), 1.05 (m, 2H, CH2), 0.88 (t, 6H, J=7.1, CH3). MS (CH₃OH) m/z 538.6 ([MH]+, 2), 498.5.6 ([MH-2H₂O]+, 10), 488.4 ([MH—(CH₃)₂CO]+, 100), 449.5 (10). Calcd for C₃₃H₆₃NO₄ m/z 537.48 [M].

### f. (2S,3R,2′S,4E)-1,3-Isopropylidene-2′-hydroxy-C12-ceramide (4c)

Prepared from 1 and dl-2-hydroxydodecanoic acid in 37% yield after purification by column chromatography (ethyl acetate-n-hexane, 2:3, v/v). Analytical sample of 4c was prepared by crystallization from ethyl acetate-n-hexane (3:1, v/v) as a white microcrystalline powder, mp 80-82° C. TLC Rf=0.40 (ethyl acetate-n-hexane, 2:3, v/v); [α]_{D}²³−6.84 (c 0.5, CHCl₃); [α] [symbol] _{S}²³−34.2 (c 0.5, CHCl₃); 1H NMR (400 MHz, CDCl₃) δ 6.21 (d, 1H, J=8.4, NH), 5.74 (dtd, 1H, J=15.0, 7.5, 1.0, 5-H), 5.40 (ddt, 1H, J=15.1, 7.5, 1.4, 4-H), 4.12, (dd, 1H, J=7.6 and 9.2, 3-H), 4.04 (q, 1H, J=4.2, 2′-HS), 3.97 (dd, 1H, J=5.2 and 11.2, 1-Ha), 3.79 (m, 1H, 2-H), 3.64 (dd, 1H, J=9.6 and 11.2, 1-Hb), 2.24 (d, 1H, J=4.4, 2′-OH), 2.0 (m, 2H, C(6)Ha), 1.92 (m, 0.5H, CH2), 1.78 (m, 1H, 3′-Ha), 1.67 (m, 0.5H, CH2), 1.58 (m, 1H, 3′-Hb), 1.48 (s, 3H, CH3), 1.42 (s, 3H, CH3), 1.24 (m, 36.5H, CH2), 1.05 (m, 0.5H, CH2), 0.88 (t, 6H, J=7.1, CH3). MS (CH3OH) m/z 538.6 ([MH]+, 2), 498.5.6 ([MH-2H2O]+, 10), 488.4 ([MH—(CH₃)₂CO]+, 100), 449.5 (10). Calcd for C₃₃H₆₃NO₄ m/z 537.48 [M].

### g. (2S,3R,2′R,4E)-1,3-isopropylidene-2′-hydroxy-C16-ceramide (3d)

Prepared from 1 and dl-2-hydroxyhexadecanoic acid in 40% yield after purification by column chromatography (ethyl acetate-n-hexane, 2:3, v/v). Analytical sample of 3d was prepared by crystallization from ethyl acetate-n-hexane (3:1, v/v) as a white microcrystalline powder, mp 100-101° C. TLC Rf=0.25 (ethyl acetate-n-hexane, 2:3, v/v); [α]_{D}²²+8.6 (c 0.8, CHCl3); [α] [symbol] ²²+17.5 (c 0.8, CHCl₃); 1H NMR (500 MHz, CDCl₃) δ 6.29 (d, 1H, J=8.6, NH), 5.74 (dtd, 1H, J=15.4, 7.6, 1.0, 5-H), 5.42 (ddt, 1H, J=15.4, 7.7, 1.4, 4-H), 4.07 (m, 2H, 2′-HR and 3-H), 3.97 (dd, 1H, J=4.4 and 11.4, 1-Ha), 3.86 (m, 1H, 2-H), 3.64 (dd, 1H, J=9.6 and 11.4, 1-Hb), 2.01 (m, 2H, C(6)Ha), 1.92 (m, 0.5H, CH2), 1.78 (m, 1H, 3′-Ha), 1.68 (m, 0.5H, CH2), 1.57 (m, 1H, 3′-Hb), 1.49 (s, 3H, CH3), 1.42 (s, 3H, CH3), 1.24 (m, 44.5H, CH2), 1.1 (m, 0.5H, CH2), 0.88 (t, 6H, J=7.1, CH3). MS (CH₃OH) m/z 616.6 ([M+Na]+, 4), 536.5 ([MH—(CH₃)₂CO]+, 100), 449.5 (85). Calcd for C₃₇H₇₁NO₄ m/z 593.54 [M].

### h. (2S,3R,2′S,4E)-1,3-Isopropylidene-2′-hydroxy-C16-ceramide (4d)

Prepared from 1 and dl-2-hydroxyhexadecanoic acid in 38% yield after purification by column chromatography (ethyl acetate-n-hexane, 2:3, v/v). Analytical sample of 4d was prepared by crystallization from ethyl acetate-n-hexane (3:1, v/v) as a white microcrystalline powder, mp 81-83° C. TLC Rf=0.44 (ethyl acetate-n-hexane, 2:3, v/v); [α]_{D}²²−7.6 (c 0.8, CHCl3); [α]₃ [symbol] _{S}²²−36.8 (c 0.8, CHCl₃); 1H NMR (500 MHz, CDCl₃) δ 6.20 (d, 1H, J=8.2, NH), 5.75 (dtd, 1H, J=15.3, 6.7, 1.0, 5-H), 5.40 (ddt, 1H, J=15.3, 6.6, 1.2, 4-H), 4.13 (dd, 1H, J=7.7 and 9.6, 3-H), 4.03 (q, 1H, J=4.1, 2′-HS), 3.98 (dd, 1H, J=5.2 and 11.2, 1-Ha), 3.80 (m, 1H, 2-H), 3.68 (dd, 1H, J=9.6 and 11.2, 1-Hb), 2.22 (d, 1H, J=4.9, 2′-OH), 2.02 (m, 2H, C(6)H2), 1.78 (m, 1H, 3′-Ha), 1.58 (m, 1H, 3′-Hb), 1.49 (s, 3H, CH3), 1.42 (s, 3H, CH3), 1.24 (m, 46H, CH2), 0.87 (t, 6H, J=7.1, CH3). MS (CH3OH) m/z 616.6 ([M+Na]+, 4), 536.5 ([MH—(CH₃)₂CO]+, 100), 449.5 (85). Calcd for C₃₇H₇₁NO₄ m/z 593.54 [M].

### i. (2S,3R,2′R)-1,3-Isopropylidene-2′-hydroxy-C6-dihydroceramide (5b)

Prepared from 2 and dl-2-hydroxyhexanoic acid in 32% yield after purification by column chromatography (ethyl acetate-n-hexane, 1:1, v/v). Analytical sample of 5b was prepared by crystallization from ethyl acetate-n-hexane (3:1, v/v) as a white microcrystalline powder, mp 61-62° C. TLC Rf=0.26 (ethyl acetate-n-hexane, 1:1, v/v); [α]_{D}²³+29.4 (c 0.5, CH₃OH); [α]_{36S}²³+93.4 (c 0.5, CH₃OH); 1H NMR (500 MHz, CDCl₃) δ 6.43 (d, 1H, J=8.4, NH), 4.14 (q, 1H, J=4.2, 2′-1-HR), 3.86 (m, 2H, 1-Ha and 3-H), 3.53 (m, 2H, 2-H and 1-Hb), 1.82 (m, 3′-Ha, 1H), 1.63 (m, 3′-Hb, 1H), 1.23-1.52 (m, 36H, CH2 and 2×CH3), 0.85 (m, 6H, 2×CH3); MS (CH₃OH) m/z 456.4 ([MH]+, 100), 398.4 ([MH—(CH₃)₂CO]+, 30). Calcd for C₂₇H₃₁NO₄ m/z 455.71 [M].

### j. (2S,3R,2′S)-1,3-Isopropylidene-2′-hydroxy-C6-dihydroceramide (6b)

Prepared from 2 and dl-2-hydroxyhexanoic acid in 35% yield after purification by column chromatography (ethyl acetate-n-hexane, 1:1, v/v). Analytical sample of 6b was prepared by crystallization from ethyl acetate-n-hexane (3:1, v/v) as a white microcrystalline powder, mp 83-84° C. TLC Rf=0.46 (ethyl acetate-n-hexane, 1:1, v/v); [α]_{D}²³+11.0 (c 0.5, CH₃OH); [α] [symbol] ²³+23.4 (c 0.5, CH₃OH); 1H NMR (500 MHz, CDCl₃) δ 6.35 (d, 1H, J=8.5, NH), 4.12 (q, 1H, J=4.1, 2′-HS), 3.90 (m, 2H, 1-Ha and 3-H), 3.55 (m, 2H, 2-H and 1-Hb), 1.82 (m, 3′-Ha, 1H), 1.60 (m, 3′-Hb, 1H), 1.20-1.52 (m, 36H, CH2 and 2×CH3), 0.90 (m, 6H, 2×CH3). MS (CH3OH) m/z 456.4 ([MH]+, 100), 398.4 ([MH—(CH3)2CO]+, 30). Calcd for C₂₇H₃₁NO₄ m/z 455.71 [M].

### iv. General procedure for the preparation of 2′-OHCers (7b-d, 8b-d) and 2′-OH—C6-dhCers (9b, 10b)

A mixture of 1,3-isopropylidene-2′-hydroxy-ceramide (0.07 mmol) and p-toluenesulfonic acid monohydrate (19 mg, 0.10 mmol) in dry methanol (2 mL) was stirred at room temperature for 4 hrs. The reaction mixture was evaporated under reduced pressure to dryness. The obtained residue was purified by flash column chromatography using the suitable solvent system.

### a. (2S,3R,2′R,4E)-2′-Hydroxy-C4-ceramide (7a)

Prepared from 3a in 80% yield after purification by column chromatography (CHCl3-CH3OH, 45:5, v/v). Analytical sample of 7a was prepared by crystallization from acetone as a white microcrystalline powder, mp 94-96° C. TLC Rf=0.32 (CHCl₃—CH₃OH, 45:5, v/v); [α]_{D}²²+18.4 (c 0.25, CHCl3); [α] [symbol] ²²+43.1 (c 0.25, CHCl₃); 1H NMR (500 MHz, CDCl3) δ 7.12 (d, 1H, J=7.7, NH), 5.78 (dtd, 1H, J=15.1, 6.8, 1.0, 5-H), 5.52 (ddt, 1H, J=15.1, 6.8, 1.1, 4-H), 4.31 (m, 1H, 3-H), 4.12 (dd, 1H, J=4.1 and 7.2, 2′-HR), 3.93 (m, 2H, 1-Ha and 2-H), 3.74 (dd, 1H, J=3.1 and 11.0, 1-Hb), 2.04 (q, 2H, J=7.0, C(6)H2), 1.87 (m, 1H, 3′-Ha), 1.70 (m, 1H, 3′-Hb), 1.39 (m, 2H, C(7)H2), 1.23 (m, 20H, CH2), 0.99 (t, 3H, J=7.1, C′H3), 0.87 (t, 3H, J=7.1, CH3); 13C NMR (CDCl3) δ 174.7 (C═O), 134.6 (C5), 128.3 (C4), 74.3 (C3), 73.2 (C′2), 62.1 (C1), 54.3 (C2), 32.3 (C′3), 31.9 (C6), 29.6, 29.4, 29.3, 29.2, 29.0, 27.6 and 22.6 (C7-C17), 14.1 (C′H3), 9.15 (CH3). (CD3OD) δ 176.8 0 (C═O), 134.78 (C5), 130.80 (C4), 73.83 (C3), 73.21 (C′2), 61.79 (C1), 55.97 (C2), 33.23 (C′3), 32.94 (C6), 30.66, 30.58, 30.52, 30.34, 30.29, 30.11, 28.59 and 23.61 (C7-C17), 14.35 (C′H3), 9.68 (CH₃) MS (CH₃OH) m/z 368.1 ([MH—H2O]+, 14), 386.0 ([MH]+, 10), 793.1 ([2M+Na]+, 100). Calcd for C₂₂H₄₉NO₄ m/z 385.32 [M].

### b. (2S,3R,2′S,4E)-2′-Hydroxy-C4-ceramide (8a)

Prepared from 4a in 81% yield after purification by column chromatography (CHCl3-CH3OH, 45:5, v/v). Analytical sample of 8a was prepared by crystallization from acetone as a white microcrystalline powder, mp 80-82° C. TLC Rf=0.34 (CHCl3-CH₃OH, 45:5, v/v); [α]_{D}²²−25.1 (c 0.25, CHCl₃), [α] [symbol] ²²−95.8 (c 0.25, CHCl₃); 1H NMR (500 MHz, CDCl3) δ 7.13 (d, 1H, J=7.4, NH), 5.82 (dtd, 1H, J=15.1, 7.0, 1.0, 5-H), 5.54 (ddt, 1H, J=15.1, 7.0, 1.1, 4-H), 4.38 (t, 1H, J=5.4, 3-H), 4.15 (dd, 1H, J=4.1 and 7.1, 2′-Hs), 4.01 (dd, 1H, J=4.0 and 11.4, 1-Ha), 3.84 (m, 1H, 2-H), 3.75 (dd, 1H, J=4.1 and 11.4, 1-Hb), 2.08 (q, 2H, J=7.0, C(6)H2), 1.91 (m, 1H, 3′-Ha), 1.74 (m, 1H, 3′-Hb), 1.41 (m, 2H, C(7)H2), 1.23 (m, 20H, CH2), 1.03 (t, 3H, J=7.1, C′H3), 0.92 (t, 3H, J=7.1, CH3); (CD3OD) d 5.70 (dtd, 1H, J=15.4, 7.0, 1.0, 5-H), 5.49 (ddt, 1H, J=15.4, 7.0, 1.0, 4-H), 4.15 (t, 1H, J=7.0, 3-H), 3.94 (dd, 1H, J=5.6 and 7.0, 2′-HS), 3.84 (m, 1H, 2-H), 3.75 (dd, J=4.9 and 11.2, 1-Ha), 3.63 (dd, 1H, J=4.2 and 11.2, 1-Hb), 2.04 (q, 2H, J=7.0, C(6)H2), 1.77 (m, 1H, 3′-Ha), 1.62 (m, 1H, 3′Hb), 1.37 (m, 2H, CH2) 1.28 (m, 42H, CH2), 0.94 (t, 6H, J=7.0, CH3), 0.89 (t, 6H, J=7.0, CH3); (CD3OD) δ 177.01 (C═O), 134.86 (C5), 130.92 (C4), 74.03 (C3), 73.36 (C′2), 62.09 (C1), 56.44 (C2), 33.55 (C′3), 33.23 (C6), 30.97, 30.96, 30.95, 30.92, 30.81, 30.49, 30.47, 28.80 and 23.89 (C7-C17), 14.58 (C′H3), 9.77 (CH3). MS (CH₃OH) m/z 385.9 ([MH]+, 28), 368.1 ([MH—H₂O]+, 25), 793.1 ([2M+Na]+, 100). Calcd for C₂₂H₄₉NO₄ m/z 385.32 [M].

### c. (2S,3R,2′R,4E)-2′-Hydroxy-C6-ceramide (7b, LCL366)

Prepared from 3b in 78% yield after purification by column chromatography (CHCl3-CH3OH, 45:5, v/v). Analytical sample of 7b was prepared by crystallization from acetone as a white microcrystalline powder, mp 85-86° C. TLC Rf=0.38 (CHCl₃—CH3OH, 45:5, v/v); [α]_{D}²²+7.7 (c 0.25, CHCl3); [α] [symbol] ₆ [symbol] ²²+28.6 (c 0.25, CHCl3); 1H NMR (500 MHz, CDCl₃) δ 7.21 (d, 1H, J=7.0, NH), 5.77 (dtd, 1H, J=15.1, 6.8, 1.0, 5-H), 5.52 (ddt, 1H, J=15.1, 6.8, 1.1, 4-H), 4.29 (t, 1H, J=5.0, 3-H), 4.13 (dd, 1H, J=3.7 and 8.2, 2′-HR), 3.92 (m, 2H, 1-Ha and 2-H), 3.75 (dd, 1H, J=5.7 and 12.0, 1-Hb), 2.05 (q, 2H, J=7.1, C(6)H2), 1.82 (m, 1H, 3′-Ha), 1.62 (m, 1H, 3-′Hb), 1.37 (m, 4H, CH2), 1.26 (m, 22H, CH2), 0.91 (t, 3H, C′H3), 0.88 (t, 3H, J=7.1, CH3); (CD3OD) δ 5.73 (dtd, 1H, J=15.1, 7.0, 1.0, 5-H), 5.47 (ddt, 1H, J=15.1, 7.0, 1.1, 4-H), 4.09 (t, 1H, J=7.0, 3-H), 3.97 (dd, 1H, J=3.8 and 8.1, 2′-HR), 3.83 (m, 1H, 2-H), 3.77 (dd, 1H, J=5.4 and 11.2, 1-Ha), 3.66 (dd, 1H, J=4.1 and 11.2, 1-Hb), 2.03 (q, 2H, J=7.1, C(6)H2), 1.74 (m, 1H, 3′-Ha), 1.53 (m, 1H, 3-′Hb), 1.4 (m, 4H, CH2), 1.28 (m, 22H, CH2), 0.91 (t, 3H, J=7.1, C′H3), 0.89 (t, 3H, J=7.1, CH3); 13C NMR (CDCl₃) δ 175.5 (C═O), 134.6 (C5), 128.3 (C4), 74.0 (C3), 72.4 (C′2), 62.0 (C1), 54.4 (C2), 34.2 (C′3), 32.3 (C6), 32.0, 29.7, 29.5, 29.3, 29.2, 29.1, 27.2, 22.7 and 22.5 (C7-C17, C′4 and C′5), 14.1 (C′H3), 13.9 (CH3); (CD3OD) δ 177.1 (C═O), 134.8 (C5), 131.0 (C4), 73.3 (C3), 72.9 (C′2), 61.8 (C1), 56.0 (C2), 35.5 (C′3), 33.4 (C6), 33.0, 30.7, 30.6, 30.4, 30.3, 30.2, 28.4, 23.7 and 23.6 (C7-C17, C′4 and C′5), 14.4 (C′H₃), 14.3 (CH₃); MS (CH3OH) m/z 436.4 ([M+Na]+, 20), 414.4 ([MH]+, 100), 396.4 ([MH—H2O]+, 10). Calcd for C₂₄H₄₇NO₄ m/z 413.35 [M].

### d. (2S,3R,2′S,4E)-2′-Hydroxy-C6-ceramide (8b, LCL367)

Prepared from 4b in 88% yield after purification by column chromatography (CHCl3-CH3OH, 45:5, v/v). Analytical sample of 8b was prepared by crystallization from acetone as a white microcrystalline powder, mp 80-82° C. TLC Rf=0.40 (CHCl3-CH3OH, 45:5, v/v); [α]_{D}²²−35.4 (c 0.25, CHCl3); [α] [symbol] ²²−124.8 (c 0.25, CHCl₃); 1H NMR (500 MHz, CDCl₃) δ 7.13 (d, 1H, J=7.7, NH), 5.80 (dtd, 1H, J=15.1, 6.8, 1.0, 5-H), 5.52 (ddt, 1H, J=15.1, 6.8, 1.1, 4-H), 4.35 (t, 1H, J=5.2, 3-H), 4.14 (dd, 1H, J=3.9 and 8.0, 2′-HR), 3.97 (dd, 1H, J=4.0 and 11.4, 1-Ha), 3.90 (m, 1H, 2-H), 3.72 (dd, 1H, J=3.5 and 11.4, 1-Hb), 2.05 (q, 2H, J=7.1, C(6)H2), 1.83 (m, 1H, 3′-Ha), 1.65 (m, 1H, 3-′Hb), 1.37 (m, 4H, CH2), 1.26 (m, 22H, CH2), 0.92 (t, 3H, J=7.1, C′H3), 0.88 (t, 3H, J=7.1, CH3); (CD3OD) δ 7.56 (d, 0.5H, J=9.0, NH), 5.70 (dtd, 1H, J=15.1, 7.0, 1.0, 5-H), 5.47 (ddt, 1H, J=15.1, 7.0, 1.1, 4-H), 4.15 (t, 1H, J=6.7, 3-H), 3.97 (dd, 1H, J=3.8 and 8.1, 2′-HS), 3.84 (m, 1H, 2-H), 3.75 (dd, 1H, J=5.4 and 11.1, 1-Ha), 3.63 (dd, 1H, J=4.4 and 11.1, 1-Hb), 2.03 (q, 2H, J=7.1, C(6)H2), 1.74 (m, 1H, 3′-Ha), 1.57 (m, 1H, 3-′Hb), 1.4 (m, 4H, CH2), 1.28 (m, 22H, CH2), 0.91 (t, 3H, C′H3), 0.89 (t, 3H, J=7.1, CH3); 13C NMR (CDCl3) δ 175.2 (C═O), 134.5 (C5), 127.9 (C4), 73.7 (C3), 72.3 (C′2), 62.2 (C1), 54.5 (C2), 34.3 (C′3), 32.3 (C6), 31.9, 29.6, 29.4, 29.3, 29.2, 29.1, 27.2, 22.6 and 22.4 (C7-C17, C′4 and C′S), 14.1 (C′H3), 13.9 (CH3); (CD3OD) δ 177.0 (C═O), 134.6 (C5), 131.7 (C4), 73.1 (C3), 72.8 (C′2), 61.9 (C1), 56.2 (C2), 35.4 (C′3), 33.3 (C6), 33.0, 30.8, 30.7, 30.6, 30.4, 30.3, 28.3, 23.7 and 23.6 (C7-C17, C′4 and C′S), 14.4 (C′H3), 14.3 (CH3); MS (MeOH) m/z 436.4 ([M+Na]+, 20), 414.4 ([MH]+, 100), 396.4 ([MH—H₂O]+, 10). Calcd for C₂₄H₄₇NO₄ m/z 413.35 [M].

### e. (2S,3R,2′R,4E)-2′-Hydroxy-C12-ceramide (7c)

Prepared from 4c in 75% yield after column chromatography purification (Silica Gel 60; CHCl3-MeOH, 45:5, v/v). Analytical sample of 7c was prepared by crystallization from acetone as a white microcrystalline powder. TLC Rf=0.37 (CHCl₃-MeOH, 45:5, v/v); [α]_{D}²³+8.4 (c 0.25, CHCl₃); [α]₃₆ [symbol] ²³+19.0 (c 0.25, CHCl3); 1H NMR (500 MHz, CD3OD-CDCl3, 3:1, v/v)/ δ 5.63 (dtd, 1H, J=15.1, 6.8, 1.0, 5-H), 5.35 (ddt, 1H, J=15.1, 6.8, 1.1, 4-H), 4.0 (t, 1H, J=5.6, 3-H), 3.95 (m, H2O and 2′-HR), 3.65 (m, 2H, 1-Ha and 2-H), 3.57 (dd, 1H, J=3.6 and 11.2, 1-Hb), 1.95 (q, 2H, J=7.1, C(6)H2), 1.65 (m, 1H, 3′-Ha), 1.44 (m, 1H, 3′-Hb), 1.15 (m, 38H, CH2), 0.76 (t, 6H, J=7.1, CH3); ESI-MS (CH3OH, relative intensity, %) m/z 498.5 ([MH]+, 100), 480.4 ([MH—H2O]+, 15). Calcd for C₃₀H₅₉NO₄ m/z 497.44 [M].

### f. (2S,3R,2′S,4E)-2′-Hydroxy-C12-ceramides (8c)

Prepared from 4c in 70% yield after column chromatography purification (Silica Gel 60; CHCl3-CH3OH, 45:5, v/v). Analytical sample of 8c was prepared by crystallization from acetone as a white microcrystalline powder. TLC Rf=0.46 (CHCl3-CH3OH, 45:5, v/v); [α]_{D}²³−26.9 (c 0.25, CHCl3); [α] [symbol] ²³−99.0 (c 0.25, CHCl₃); 1H NMR (500 MHz, CDCl₃) δ 7.30 (d, 1H, J=8.4, NH), 5.70 (dtd, 1H, J=15.1, 6.8, 1.0, 5-H), 5.40 (ddt, 1H, J=15.1, 6.8, 1.1, 4-H), 4.17 (m, 1H, 3-H), 3.93 (dd, 1H, J=4.0 and 8.4, 2′-HS), 3.72 (m, 2H, 1-Ha and 2-H), 3.53 (dd, 1H, J=5.6 and 13.2, 1-Hb), 1.96 (q, 2H, J=7.0, C(6)H2), 1.72 (m, 1H, 3′-Ha), 1.48 (m, 1H, 3′-Hb), 1.20 (m, 38H, CH2), 0.79 (t, 6H, J=7.1, CH3). ESI-MS (CH3OH, relative intensity, %) m/z. 498.5 ([MH]+, 100), 480.4 ([MH—H₂O]+, 15). Calcd for C₃₀H₅₉NO₄ m/z 497.44 [M].

### g. (2S,3R,2′R,4E)-2′-Hydroxy-C16-ceramide (7d)

Prepared from 3d in 88% yield after column chromatography purification as a white microcrystalline powder (Silica Gel 60; CHCl3-CH₃OH, 45:5, v/v). Analytical sample of 7d was prepared by crystallization from acetone as a white microcrystalline powder, mp 101-103° C. TLC Rf=0.38 (CHCl3-CH3OH, 45:5, v/v); [α] [symbol] ²³+5.2 (c 0.25, MeOH—CHCl₃, 2:8, v/v); [α] [symbol] _{S}²³+32.2 (c 0.25, CH₃OH—CHCl₃, 2:8, v/v); 1H NMR (500 MHz, CD3OD-CDCl3, 1:3, v/v) δ 5.76 (dtd, 1H, J=15.1, 6.8, 1.0, 5-H), 5.47 (ddt, 1H, J=15.1, 6.8, 1.1, 4-H), 4.12 (t, 1H, J=6.0, 3-H), 4.01 (dd, 1H, J=3.7 and 8.2, 2′-HR), 3.85 (m, 1H, 2-H), 3.80 (dd, J=8.6 and 11.3, 1-Ha), 3.68 (dd, 1H, J=3.7 and 11.3, 1-Hb), 2.05 (q, 2H, J=7.1, C(6)H2), 1.78 (m, 1H, 3′-Ha), 1.58 (m, 1H, 3′Hb), 1.26 (m, 46H, CH2), 0.88 (t, 6H, J=7.1, CH3); (CDCl3) δ 7.11 (d, 1H, J=8.2, NH) 5.78 (dtd, 1H, J=15.1, 6.8, 1.0, 5-H), 5.52 (ddt, 1H, J=15.1, 6.8, 1.1, 4-H), 4.30 (t, 1H, J=6.0, 3-H), 4.13 (dd, 1H, J=3.5 and 8.0, 2′-HR), 3.93 (m, 2H, 1-Ha and 2-H), 3.74 (dd, 1H, J=5.7 and 13.0, 1-Hb), 2.05 (q, 2H, J=7.1, C(6)H2), 1.82 (m, 1H, 3′-Ha), 1.64 (m, 1H, 3′-Hb), 1.26 (m, 46H, CH2), 0.88 (t, 6H, J=7.1, CH3); (CD3OD, 30° C., 700 MHz) d 5.72 (dtd, 1H, J=15.3, 7.0, 0.9, 5-H), 5.47 (ddt, 1H, J=15.3, 7.3, 0.9, 4-H), 4.08 (t, 1H, J=7.4, 3-H), 4.12 (dd, 1H, J=3.8 and 7.9, 2′-HR), 3.84 (m, 1H, 2-H), 3.76 (dd, J=4.9 and 11.2, 1-Ha), 3.64 (dd, 1H, J=5.0 and 11.2, 1-Hb), 2.05 (q, 2H, J=7.0, C(6)H2), 1.76 (m, 1H, 3′-Ha), 1.57 (m, 1H, 3′Hb), 1.40 (m, 4H, CH2) 1.29 (m, 42H, CH2), 0.90 (t, 6H, J=7.1, CH3); 13C NMR (CDCl₃) δ 174.7 (C═O), 134.5 (C5), 128.7 (C4), 74.4 (C3), 72.46 (C′2), 62.3 (C1), 54.6 (C2), 34.9 (C′3), 32.3 (C6), 31.9, 29.7, 29.5, 29.4, 29.3, 29.2, 29.1, 25.0 and 22.7 (C7-C17 and C′4-C′15), 14.0 (C′H3 and CH3); (CD3OD) δ 177.3 (C═O), 135.0 (C5), 131.2 (C4), 73.4 (C3), 73.2 (C′2), 62.1 (C1), 56.1 (C2), 36.0 (C′3), 33.6 (C6), 33.2, 30.98, 30.96, 30.94, 30.92, 30.9, 30.88, 30.82, 30.7, 30.62, 30.61, 30.49), 26.3 and 23.8 (C7-C17 and C′4-C′15), 14.5 (C′H3 and CH3). ESI-MS (CH3OH, relative intensity, %) m/z 1130.5 and 1129.4 ([2M+Na]+ and ([2M−H+Na]+, 70 and 100), 1107.0 ([2M+H]+, 35), 554.1 (MH+, 16), 536.3 ([MH—H₂O]+, 27). Calcd for C₃₄H₆₇NO₄ m/z 553.51 [M].

### h. (2S,3R,2′S,4E)-2′-Hydroxy-C16-ceramides (8d)

Prepared from 4d in 81% yield after column chromatography purification as a white microcrystalline powder (Silica Gel 60; CHCl3-MeOH, 45:5, v/v). Analytical sample of 8d was prepared by crystallization from acetone as a white microcrystalline powder, mp 100-102° C. TLC Rf=0.39 (CHCl₃—CH3OH, 45:5, v/v); [α]₀²³−16.1 (c 0.25, MeOH—CHCl3, 2:8, v/v); [α] [symbol] ²³−50.7 (c 0.25, CH3OH—CHCl3, 2:8, v/v); 1H NMR (500 MHz, CD3OD-CDCl3, 1:3, v/v) δ 7.42 (d, ˜0.5H, J=8.2, NH), 5.76 (dtd, 1H, J=15.1, 6.8, 1.0, 5-H), 5.47 (ddt, 1H, J=15.1, 6.8, 1.1, 4-H), 4.22 (t, 1H, J=6.3, 3-H), 4.01 (dd, 1H, J=3.8 and 8.3, 2′-HS), 3.85 (m, 1H, 2-H), 3.79 (dd, 1H, J=8.2 and 11.2, 1-Ha), 3.62 (dd, 1H, J=5.6 and 11.2, 1-Hb), 2.04 (q, 2H, J=7.0, C(6)H2), 1.78 (m, 1H, 3′-Ha), 1.58 (m, 1H, 3′-Hb), 1.27 (m, 46H, CH2), 0.88 (t, 6H, J=7.1, CH3); (CDCl3) δ 7.10 (d, 1H, J=8.2, NH) 5.80 (dtd, 1H, J=15.1, 6.8, 1.0, 5-H), 5.53 (ddt, 1H, J=15.1, 6.8, 1.1, 4-H), 4.34 (t, 1H, J=5.0, 3-H), 4.12 (dd, 1H, J=4.0 and 8.0, 2′-HR), 3.95 (dd, J=4.0 and 11.5, 1-Ha), 3.89 (m, 1H, 2-H), 3.72 (dd, 1H, J=3.5 and 11.5, 1-Hb), 2.06 (q, 2H, J=7.0, C(6)H2), 1.82 (m, 1H, 3′-Ha), 1.64 (m, 1H, 3′Hb), 1.26 (m, 46H, CH2), 0.88 (t, 6H, J=7.1, CH3); (CD3OD, 30° C., 700 MHz) d 5.73 (dtd, 1H, J=15.3, 7.0, 1.0, 5-H), 5.49 (ddt, 1H, J=15.3, 7.0, 1.0, 4-H), 4.16 (t, 1H, J=6.3, 3-H), 4.12 (q, 1H, J=5.6, 2′-HS), 3.84 (m, 1H, 2-H), 3.79 (dd, J=5.1 and 11.2, 1-Ha), 3.64 (dd, 1H, J=4.0 and 11.2, 1-Hb), 2.04 (q, 2H, J=7.0, C(6)H2), 1.71 (m, 1H, 3′-Ha), 1.55 (m, 1H, 3′Hb), 1.37 (m, 4H, CH2) 1.29 (m, 42H, CH2), 0.90 (t, 6H, J=7.1, CH3); 13C NMR (CDCl3) δ 174.6 (C═O), 134.5 (C5), 128.5 (C4), 74.2 (C3), 72.38 (C′2), 62.4 (C1), 54.7 (C2), 35.0 (C′3), 32.9 (C6), 31.9, 29.7, 29.6, 29.54 29.5, 29.4, 29.3, 29.2, 29.0, 25.0 and 22.6 (C7-C17 and C′4-C′15), 14.0 (C′H3 and CH3); (CD3OD) δ 177.2 (C═O), 134.9 (C5), 130.9 (C4), 73.3 (C3), 73.03 (C′2), 62.1 (C1), 56.5 (C2), 30.98, 30.96, 30.94, 30.92, 30.9, 30.88, 30.82, 30.7, 30.62, 30.61, 30.49, 26.2 and 23.9 (C7-C17 and C′4-C′15), 14.5 (C′H3 and CH3).

### i. (2S,3R,2′R)-2′-Hydroxy-C6-dihydroceramide (9b, LCL447)

Prepared from 5b in 93% yield after column chromatography purification as a white microcrystalline powder (Silica Gel 60; CHCl3-CH3OH, 10:1, v/v). Analytical sample of 9b was prepared by crystallization from acetone as a white microcrystalline powder, mp 112-114° C. TLC Rf=0.24 (CHCl3-CH₃OH, 45:5, v/v); [α]₀²³+38.20 (c 0.125, MeOH); [α] [symbol] _{S}²³+124.91 (c 0.125, CH3OH); 1H NMR (500 MHz, CDCl₃) δ 7.28 (d, 1H, J=8.4, NH), 4.12 (q, 1H, J=3.6, 2′-HS), 3.95 (dd, 1H, J=3.6 and 11.2, 1-Ha), 3.82 (m, 2H, 2-H and 3-H), 3.78 (m, 1H, J=3.1 and 11.2, 1Hb), 1.82 (m, 1H, 3′-Ha), 1.61 (m, 1H, 3′-Hb), 1.51 (m, 2H, C(4)H2), 1.25 (m, 36H, CH2), 0.90 (t, 3H, J=7.1, C′H3), 0.87 (t, 3H, J=7.1, CH3). MS (CH₃OH) m/z 416.4 ([MH]+, 100), 498.3 ([MH—H2O]+, 4). Calcd for C₂₄H₄₉NO₄ m/z 415.37 [M].

### j. (2S,3R,2′S)-2′-Hydroxy-C6-dihydroceramide (10b, LCL446)

Prepared from 6b in 95% yield after column chromatography purification as a white microcrystalline powder (Silica Gel 60; CHCl3-CH3OH, 10:1, v/v). Analytical sample of 10b was prepared by crystallization from acetone as a white microcrystalline powder, mp 90-91° C. TLC Rf=0.33 (CHCl3-CH3OH, 45:5, v/v); [α]₀²³−17.20 (c 0.125, CH3OH); [α] [symbol] _{S}²³−62.03 (c 0.125, MeOH); 1H NMR (500 MHz, CDCl₃) δ 7.28 (d, 1H, J=8.4, NH), 4.14 (q, 1H, J=3.8, 2′-HS), 4.02 (dd, 1H, J=3.5 and 11.4, 1-Ha), 3.82 (m, 2H, 2-H and 3-H), 3.78 (m, 1H, J=3.1 and 11.4, 1Hb), 1.86 (m, 3′-Ha, 1H), 1.68 (m, 3′-Hb, 1H), 1.54 (m, 2H, C(4)H2), 1.25 (m, 36H, CH2), 0.90 (t, 3H, J=7.1, C′H3), 0.86 (t, 3H, J=7.1, CH3). MS (CH3OH) m/z 416.4 ([MH]+, 100), 498.3 ([MH—H2O]+, 4). Calcd for C₂₄H₄₉NO₄ m/z 415.37 [M].

### 6. Example 6

### Biological Data

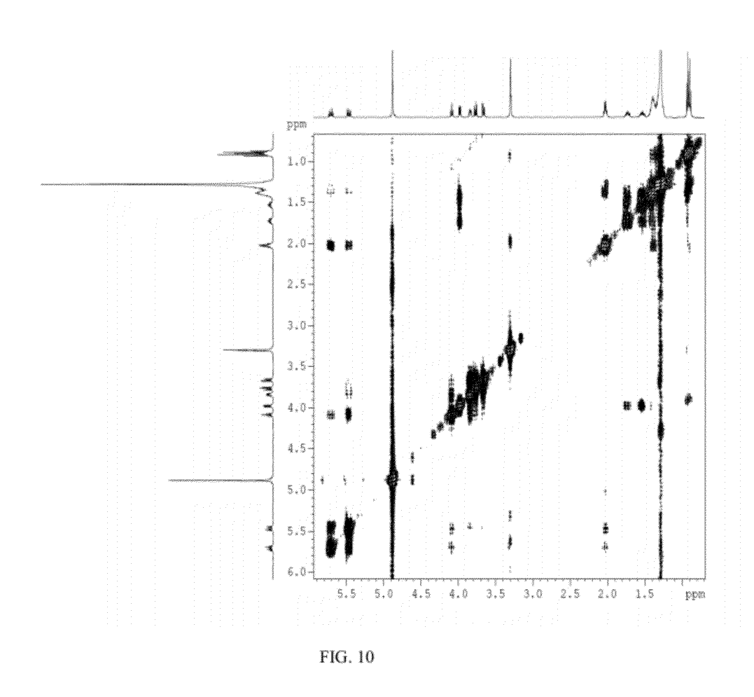

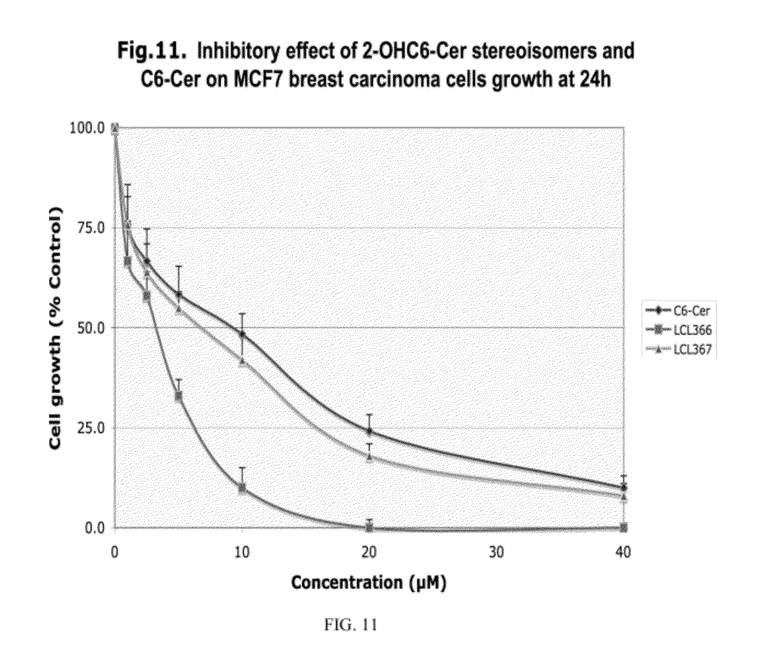

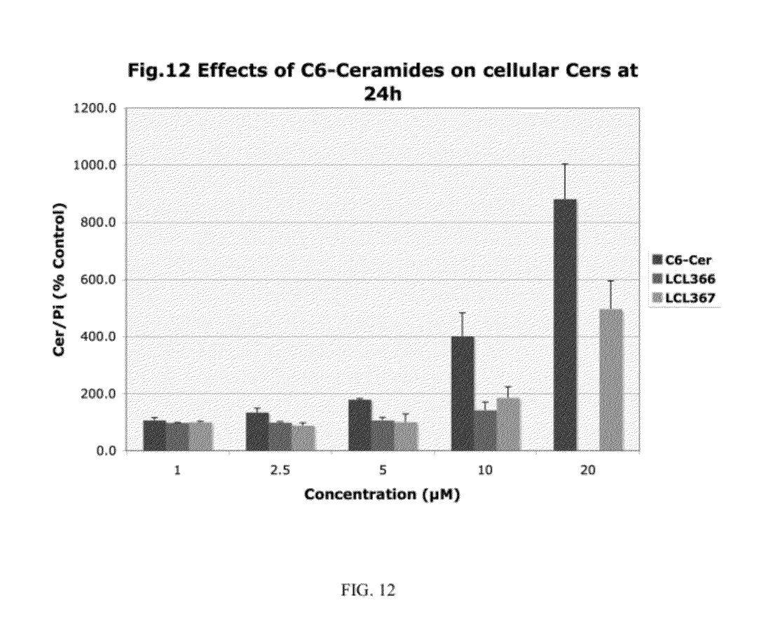

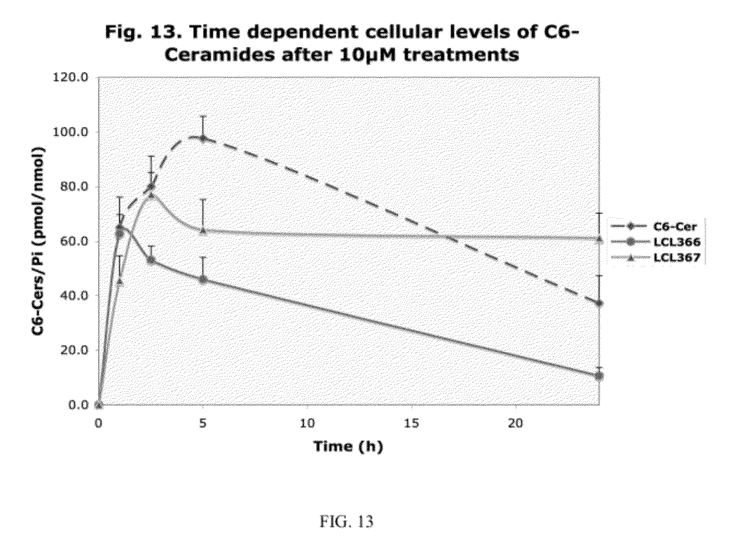

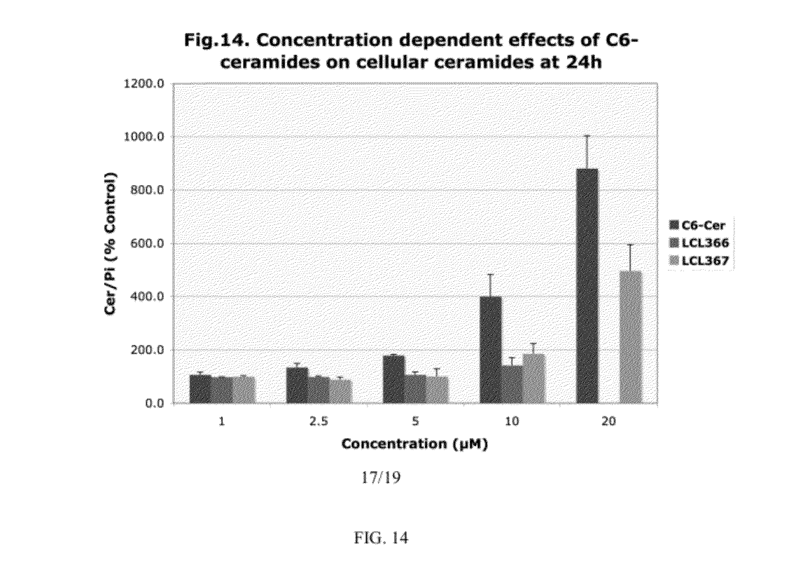

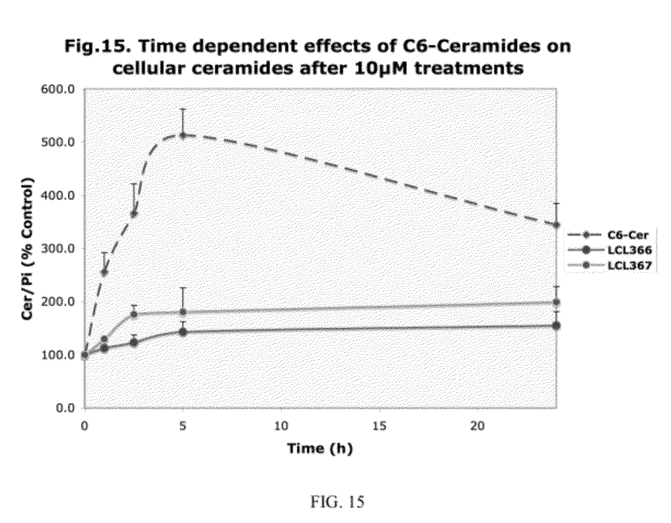

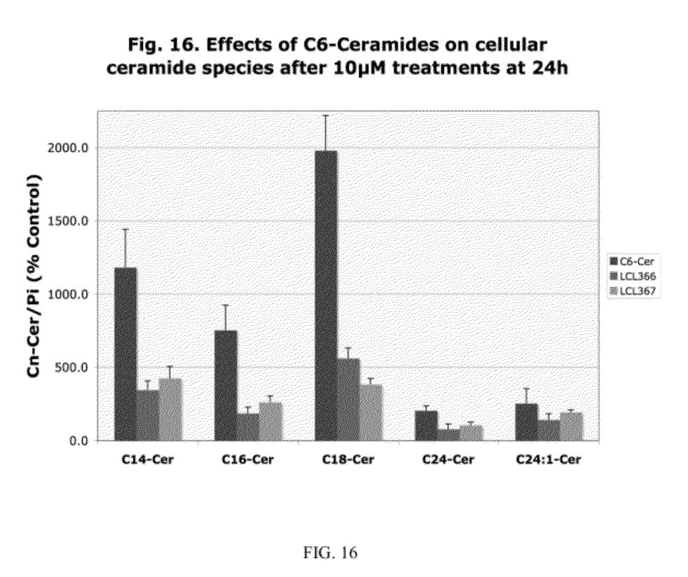

Significant differences in cytotoxicity and metabolism were observed when MCF7 cells were treated with 2′-OH—C6-Cer stereoisomers (see FIGS. 11-16). The (2′R)-isomer of 2′-OH—C6-Cer showed a significantly higher cytotoxicity (1050/24 h=3 μM), a low cellular accumulation, and no increase of cellular Cers. The (2′S)-isomer was less toxic (1050/24 h=8 μM) than the (2′R)-isomer but more potent then C6-Cer (1050/24 h=12 μM). This isomer showed a higher cellular accumulation and increased the endogenous Cers similarly as C6-Cer but with a lower potency. Interestingly, neither isomer of 2′-OH—C6-Cers and 2′-OH—C6-dhCers was metabolized to their cellular long chain 2′-OH-homologs. Most importantly, the most active (2′R)-isomers did not influence the levels of the cellular Cers nor dhCers. Contrary to this, the ordinary C6-Cer and C6-dhCer significantly increased the levels of their cellular long chain homologs. These divergent anabolic responses and SAR data indicate that (2′R)-2-OHCers/dhCers interact with distinct cellular regulatory targets in a specific and more effective manner than their non-hydroxylated analogs. These findings are important in the development of anticancer modalities which can render cancer cells apoptotic and/or inhibiting tumor growth. However, it can be even more beneficial when (2′R)-2′-OH-ceramide or its analog based drug will only target processes specifically related to the prevention of tumor growth and metastasis by inhibiting the development of the tumor vasculature (Bansode, R. R., et. al., Int. J. Biol. Sci., 7, (2011), p. 629). Since the (2′R)-isomers of 2′-OH-Cer disclosed herein are not increasing the cellular Cers, in that context, their use in vivo need not induce apoptosis, so they will not have any deleterious side effects associated with inducing other non-cancerous cells into the apoptotic pathway.

The presented data indicate that observed divergence in structure and biological activity between (2′R)-isomer of 2′-OH-Cer and C6-Cer could be the result of many factors, namely those related to the differences in their electronic structure, distinctions in molecular recognition by metabolic and regulatory enzymes, site-specific delivery as well as stability under varied intracellular conditions.

### REFERENCES

1 L. M. Obeid, C. M. Linardic, L. A. Karolak and Y. A. Hannun, Science 259 (1993), p. 1769.
2 M. Mimeault, FEBS Lett. 530 (2002), p. 9.
3 A. Carpinteiro, C. Dumitru, M. Schenck and E. Gulbins, Cancer Lett. 264 (2008), p. 1.
4 K. Kitatani, J. Idkowiak-Baldys and Y. H. Hannun, Cell. Signalling 20 (2008), p. 1010.
5 http://www.lipidmaps.org/.
6 M. Inagaki, R. Isobe, Y. Kawano, T. Maiyamato, T. Komori and R. Higuchi, Eur. J. Chem. (1998), p. 129.
7 N. Asai, N. Fusetani and S. Matsunaga, J. Nat. Prod. 64 (2001), p. 1210.
8 Y. Masuda, M. Yoshida and K. Mori, Biosci. Biotechnol. Biochem. 66 (2002), p. 1531.
9 H. Azuma, R. Takao, H. Niiro, K. Shikata, S. Tamagaki, T. Tachibana and K. Ogino, J. Org. Chem. 68 (2003), p. 2790.
10 N. Q. Tien, P.-H. Ngoc, P. H. Minh, P. V. Kiem, C. V. Minh and Y. H. Kim, Arch. Pharm. Res. 27 (2004), p. 1020.
11 P. Radhika, V. L. Rao and H. Laatsch, Chem. Pharm. Bull. 52 (2004), p. 1345.
12 Y. Masuda and K. Mori, Eur. J. Org. Chem. (2005), p. 4789.
13 F. Leon, I. Brouard, A. Rivera, F. Torres, S. Rubio, J. Quintana, F. Estevez and J. Bermejo, J. Med. Chem. 49 (2006), p. 5830.
14 T. Ishii, T. Okino and Y. Mino, J. Nat. Prod. 69 (2006), p. 1080.
15 W. M. Holleran, T. Takagi and Y. Uchida, FEBS Lett. 580 (2006), p. 545.
16 A. H. Futerman and G. van Meer, Nat. Rev. Mol. Cell. Biol. 5 (2004), p. 554.
17 M. Iwamori, Human Cell 18 (2005), p. 117. View Record in Scopus
18 H. Hama, Biochim. Biophys. Acta 1801 (2010), p. 405. Article
19 M. Sugita, M. Iwamori, J. Evans, R. H. McCluer, J. T. Dulanej and H. W. Moser, J. Lipid Res. 15 (1974), p. 223.
20 M. Iwamori and W. H. Moser, Clin. Chem. 21 (1975), p. 725.
21 S. Edwardson, H. Hama, A. Shaag, J. M. Gomori, I. Berger, D. Soffer, S. H. Korman, I. Taustein, A. Saada and O. Elpeleg, Am. J. Hum. Genet. 83 (2008), p. 643.
22 K. Kiguchi, Y. Iwamori, N. Suzuki, Y. Kobayashi, B. Ishizuka, I. Ishiwata, T. Kita, Y. Kikuchi and M. Iwamori, Cancer Sci. 97 (2006), p. 1321.
23 S. A. Richtie, P. W. K. Ahiahonu, D. Jayasinghe, D. Heath, J. Liu, Y. Lu, W. Jin, A. Kavianpour, Y. Yamazaki, A. M. Khan, M. Hossain, K. K. Su-Myat, P. L. Wood, K. Krenitsky, I. Takemasa, M. Miyake, M. Sekimoto, M. Monden, H. Matsubara, F. Nomura and D. B. Goodenowe, BMC Med. 8 (2010), p. 13.
24 K. Karlsson, K. Nilsson, B. E. Samuelsson and G. O. Steen, Biochim. Biophys. Acta 176 (1969), p. 660.
25 O. Nilsson and L. Svenerholm, J. Lipid Res. 23 (1982), p. 327.
26 L. Riboni, D. Acquotti, R. Casellato, R. Ghidoni, G. Montagnolo, A. Benevento, L. Zecca, F. Rubino and S. Sonnino, Eur. J. Biochem. 201 (1992), p. 107.
27 R. Dahiya and T. A. Brasitus, Biochim. Biophys. Acta 875 (1986), p. 220. Abstract
28 Y. Kishimoto, H. Akanuma and I. Singh, Mol. Cell. Biochem. 254 (1979), p. 1050.
29 Y. Hirabayashi, A. Hamaoka, M. Matsumoto and K. Nishimura, Lipids 21 (1986), p. 710.
30 M. Kojima, M. Ohnishi and S. Ito, J. Agric. Food. Chem. 39 (1991), p. 1709.
31 P. W. Wertz and B. van der Bergh, Chem. Phys. Lipids 91 (1998), p. 85.
32 M. Ponec, A. Weerheim, P. Lankhorst and P. Wertz, J. Invest. Dermatol. 120 (2003), p. 581.
33 J. A. Bouwstra, S. G. Gooris, F. E. R. Dubbelaar and M. Ponec, J. Lipid Res. 42 (2001), p. 1759.
34 I. Pascher, Biochem. Biophys. Acta 455 (1976), p. 433.
35 H. Lofgren and I. Pasher, Chem. Phys. Lipids 20 (1977), p. 273.
36 E. Yasugi, T. Kasama and Y. Seyama, J. Biochem. 110 (1991), p. 202.
37 M. Minamino, I. Sakaguch, T. Naka, N. Ikeda, Y. Kato, I. Tomiyasu, I. Yano and K. Kobayashi, Microbiology 149 (2003), p. 2071.
38 M. Kyogasima, K. Tadano-Aritomi, T. Aoyama, A. Yusa, Y. Goto, K. Tomiya-Kozumi, H. Ito, T. Murate, R. Kannagi and A. Hara, J. Biochem. 144 (2008), p. 95.
39 H. Azuma, R. Takao, K. Shikata, H. Niiro, T. Tachibana and K. Ogino, J. Med. Chem. 46 (2003), p. 3445.
40 X. Li, D.-D. Sun, J.-W. Chen, L.-W. He, H-Q. Zhang and H.-Q. Xu, Fitoterapia 78 (2007), p. 490.
41 N. L. Alderson, B. M. Rembiesa, M. D. Walla, A. Bielawska, J. Bielawski and H. Hama, J. Biol. Chem. 279 (2004), p. 48562.
42 Y. Uchida, H. Hama, N. L. Alderson, S. Douangpanya, Y. Wang, D. A. Crumrine, P. M. Elias and W. M. Holleran, J. Biol. Chem. 282 (2007), p. 13211.
43 K. J. Dick, M. Eckhardt, C. Paisán-Ruiz, A. A. Alshehhi, C. Proukakis, N. A. Sibtain, H. Maier, R. Sharifi, M. A. Patton, W. Bashir, R. Koul, S. Raeburn, V. Gieselmann, H. Houlden and A. H. Crosby, Hum. Mutat. 31 (2010), p. E1251.
44 Y. Sun, D. P. Witte, M. Zamzow, M. Ran, B. Quinn, J. Matsuda and G. A. Grabowski, Hum. Mol. Genet. 16 (2007), p. 957.
45 J. H. Park and E. H. Schuchman, Biochem. Biophys. Acta 1758 (2006), p. 2133.
46 P. Van der Bijl, G. J. Strous, M. Lopes-Cardozo, J. Thomas-Oates and G. van Meer, Biochem. J. 317 (1996), p. 589.
47 I. Pascher and S. Sundell, Chem. Phys. Lipids 20 (1977), p. 175.
48 J. M. Boggs, K. M. Koshy and G. Rangaraj, Biochim. Biophys Acta 938 (1988), p. 361.
49 V. N. Odinkov, I. V. Guliyautinov, D. V. Nedopekin, N. A. Veskina and L. M. Khalilov, Russ. J. Org. Chem. 39 (2003), p. 952.
50 S. E. Bode, M. Muller and M. Wolberg, Org. Lett. 4 (2002), p. 619.
51 J. H. Zhu, R.-M. Yu, L. Yang and W.-M. Li, Fitoterapia 81 (2010), p. 339.
52 V. Costantino, M. D'Esposito, E. Fattorusso, A. Mangoni, N. Basilico, S. Parapini and D. Taramelli, J. Med. Chem. 48 (2005), p. 7411.
53 J. H. Jung, C.-O. Lee, Y. C. Kim and S. S. Kang, J. Nat. Prod. 59 (1996), p. 319.
54 L. Li, X. Tang, K. G. Taylor, D. B. DuPre and M. C. Yappert, Biophys. J. 82 (2002), p. 2067.
55 Z. M. Szulc, J. Bielawski, H. Gracz, M. Gustilo, N. Mayroo, Y. Hannun, L. M. Obeid and A. Bielawska, Bioorg. Med. Chem. 14 (2006), p. 7083.
56 R. J. Abraham, M. Mobil and R. J. Smith, Magn. Reson. Chem. 41 (2003), p. 26.
57 I. Sultan, C. E. Senkal, S. Ponnusamy, J. Bielawski, Z. M. Szulc, A. Bielawska, Y. A. Hannun and B. Ogretmen, Biochem. J. 393 (2006), p. 513.
58 J. V. Chapman, V. Gouaze-Andersson, M. C. Messner, M. Flowers, R. Karimi, M. Kester, B. M. Barth, X. Liu, A. E. Giulano and M. C. Cabot, Biochem. Pharmacol. 80 (2010), p. 308.
59 O. Hwang, G. Kim, Y. O. Jong, S. W. Kim, G. Choi, H. J. Choi, S. Y. Jeon, D. G. Lee and J. D. Lee, Mol. Pharmacol. 59 (2001), p. 1249.
60 A. Bielawska, H. M. Crane, D. Liotta, L. M. Obeid and Y. A. Hannun, J. Biol. Chem. 268 (1993), p. 26226.
61 B. Ogretmen, B. J. Pettus, M. J. Rossi, R. Wood, J. Usta, Z. Szulc, A. Bielawska, L. M. Obeid and Y. A. Hannun, J. Biol. Chem. 277 (2002), p. 12960.
62 E. Bieberich, Future Lipidol. 3 (2008), p. 273.
63 B. Ogretmen and Y. A. Hannun, Nat. Rev. Cancer 4 (2004), p. 604.
64 I.-N. Park, I. Cho and S. G. Kim, Drug Metab. Dispos. 9 (2004), p. 893.
65 C. L. Chik, B. Li, E. Karpinski and A. K. Ho, Mol. Cell. Endicronol. 218 (2004), p. 175.
66 N. D. Ridgeway and D. J. Merriam, Biochim. Biophys. Acta 1256 (1995), p. 57.
67 S. El-Bawab, J. Usta, P. Roddy, Z. M. Szulc, A. Bielawska and Y. A. Hannun, J. Lipid Res. 43 (2002), p. 141.
68 Y. Mizutani, A. Kihara, H. Chiba, H. Tojo and Y. Igarashi, J. Lipid Res. 49 (2008), p. 2356.
69 A. Mukhopadhyay, S. A. Saddoughi, P. Song, I. Sultan, S. Ponnusamy, C. E. Senkal, C. F. Snook, H. K. Arnoki, R. C. Sears, Y. A. Hannun and B. Ogretmen, FASEB J. 23 (2009), p. 751.
70 J. Bielawski, Z. M. Szulc, Y. A. Hannun and A. Bielawska, Methods 39 (2006), p. 82.
71 J. Bielawski, J. S. Pierce, J. Snider, B. Rembiesa, Z. M. Szulc and A. Bielawska, Methods Mol. Biol. 579 (2009), p. 443.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a ceramide” includes a plurality of such ceramides, reference to “the ceramide” is a reference to one or more ceramides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

We claim:

1. A compound having the structure: 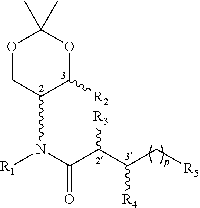
wherein:
R₁ is H or C₁-C₃ alkyl;
R₂ is selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH;
R₃ is selected from the group consisting of H, hydroxyl, halo, amino, nitro, C₁-C₃ alkoxy and keto;
R₄ is selected from the group consisting of H, hydroxyl, halo, amino, nitro, C₁-C₃ alkoxy and keto;
R₅ is selected from the group consisting of H, hydroxyl, halo, —N⁺-group; and
p is 0-30.

2. The compound of claim 1, wherein the compound is a pure isomer.

3. The compound of claim 2, wherein the pure isomer is selected from the group consisting of (2S,3R,2′R), (2S,3R,2′S), (2R,3R,2′R), (2R,3R,2′S), (2S,3S,2′R), (2S,3S,2′S), (2R,3S,2′R), (2R,3S,2′S); (2S,3R,3′R), (2S,3R,3′S), (2R,3R,3′R), (2R,3R,3′S), (2S,3S,3′R), (2S,3S,3′S), (2R,3S,3′R) and (2R,3S,3′S).

4. The compound of claim 2, wherein R₁ is H; and R₃ is hydroxyl.

5. The compound of claim 1, wherein R₁ is H;
R₂ is selected from the group consisting of C₁₅ alkyl, C₁₅ alkenyl and C₁₅ alkynyl;
R₃ is hydroxyl; and
R₄ is H.

6. A method of treating a cancer, comprising administering to a subject a therapeutically effective amount of one or more compounds of formula II: 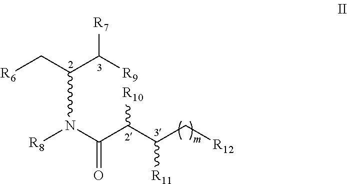
wherein:
R₆ is selected from the group consisting of H, hydroxyl, C₁-C₃ alkoxy, amino, thio, —OP(O)(OH)₂ and —PP(O)₂OR₁₃NR₁₄;
R₁₃ is C₁-C₃ alkyl;
R₁₄ is (C₁-C₃ alkyl)₃;
R₇ is selected from the group consisting of H, hydroxyl, C₁-C₃ alkoxy and amino;
R₈ is selected from the group consisting of H and C₁-C₃ alkyl;
R₉ is selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH;
R₁₀ is selected from the group consisting of H, hydroxyl, halo, amino, nitro, C₁-C₃ alkoxy and keto;
R₁₁ is selected from the group consisting of H, hydroxyl, halo, amino, nitro, C₁-C₃ alkoxy and keto;
R₁₂ is selected from the group consisting of H, hydroxyl, halo, —N⁺-group; and
m is 0-30,
or a pharmaceutically acceptable salt or ester thereof.

7. The method of claim 6, wherein the formula II is a pure isomer.

8. The method of claim 7, wherein the formula II is a pure isomer is selected from the group consisting of (2S,3R,2′R), (2S,3R,2′S), (2R,3R,2′R), (2R,3R,2′S), (2S,3S,2′R), (2S,3S,2′S), (2R,3S,2′R), (2R,3S,2′S); (2S,3R,3′R), (2S,3R,3′S), (2R,3R,3′R), (2R,3R,3′S), (2S,3S,3′R), (2S,3S,3′S), (2R,3S,3′R) and (2R,3S,3′S).

9. The method of claim 8, wherein formula II is a non-natural compound.

10. The method of claim 6, wherein R₆ is hydroxyl, R₈ is H; and R₁₀ is hydroxyl.

11. The method of claim 6, wherein R₈ is H;
R₉ is selected from the group consisting of C₁₅ alkyl, C₁₅ alkenyl and C₁₅ alkynyl;
R₁₀ is hydroxyl; and
R₁₁ is H.

12. The method of claim 6, wherein the cancer is breast cancer or lung cancer.

13. The method of claim 6, wherein the subject has been diagnosed with cancer.

14. A compound synthesized using a method comprising:
(a) condensation of a 2-hydroxyl carboxylic acid with an acetonide; and
(b) deprotecting the acetonide.

15. The compound of claim 14, wherein the intermediate is isolated after step (a).

16. The compound of claim 14, wherein the compound has the structure of formula II: 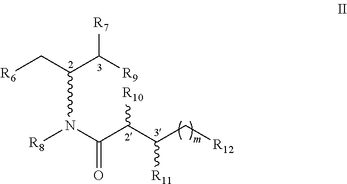
wherein:
R₆ is selected from the group consisting of H, hydroxyl, C₁-C₃ alkoxy, amino, thio, —OP(O)(OH)₂ and —OP(O)₂OR₁₃NR₁₄;
R₁₃ is C₁-C₃ alkyl;
R₁₄ is (C₁-C₃ alkyl)₃;
R₇ is selected from the group consisting of H, hydroxyl, C₁-C₃ alkoxy and amino;
R₈ is selected from the group consisting of H and C₁-C₃ alkyl;
R₉ is selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH;
R₁₀ is selected from the group consisting of H, hydroxyl, halo, amino, nitro, C₁-C₃ alkoxy and keto;
R₁₁ is selected from the group consisting of H, hydroxyl, halo, amino, nitro, C₁-C₃ alkoxy and keto;
R₁₂ is selected from the group consisting of H, hydroxyl, halo, —N⁺-group; and
m is 0-30.

17. The method of claim 16, wherein the formula II is a pure isomer is selected from the group consisting of (2S,3R,2′R), (2S,3R,2′S), (2R,3R,2′R), (2R,3R,2′S), (2S,3S,2′R), (2S,3S,2′S), (2R,3S,2′R), (2R,3S,2′S); (2S,3R,3′R), (2S,3R,3′S), (2R,3R,3′R), (2R,3R,3′S), (2S,3S,3′R), (2S,3S,3′S), (2R,3S,3′R) and (2R,3S,3′S).

18. A method of making a pure isomer comprising:
(a) condensation of a 2-hydroxyl carboxylic acid with an acetonide;
(b) isolating pure isomers.

19. The method of claim 18, further comprising deprotecting the acetonide.

20. The method of claim 48, wherein the pure isomer is selected from the group consisting of (2S,3R,2′R), (2S,3R,2′S), (2R,3R,2′R), (2R,3R,2′S), (2S,3S,2′R), (2S,3S,2′S), (2R,3S,2′R), (2R,3S,2′S); (2S,3R,3′R), (2S,3R,3′S), (2R,3R,3′R), (2R,3R,3′S), (2S,3S,3′R), (2S,3S,3′S), (2R,3S,3′R) and (2R,3S,3′S); and the pure isomer has the structure of formula II: 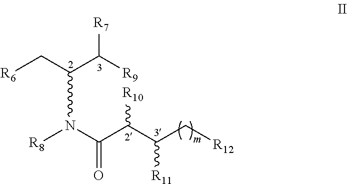
wherein:
R₆ is selected from the group consisting of H, hydroxyl, C₁-C₃ alkoxy, amino, thio, —OP(O)(OH)₂ and —OP(O)₂OR₁₃NR₁₄;
R₁₃ is C₁-C₃ alkyl;
R₁₄ is (C₁-C₃ alkyl)₃;
R₇ is selected from the group consisting of H, hydroxyl, C₁-C₃ alkoxy and amino;
R₈ is selected from the group consisting of H and C₁-C₃ alkyl;
R₉ is selected from the group consisting of C₁-C₃₀ alkyl, C₁-C₃₀ alkenyl, C₁-C₃₀ alkynyl, C₁-C₃₀ alkyl-aryl, C₂-C₃₀ alkenyl-aryl, C₁-C₃₀ alkyl-N⁺-group and C₁-C₃₀ alkyl-OH;
R₁₀ is selected from the group consisting of H, hydroxyl, halo, amino, nitro, C₁-C₃ alkoxy and keto;
R₁₁ is selected from the group consisting of H, hydroxyl, halo, amino, nitro, C₁-C₃ alkoxy and keto;
R₁₂ is selected from the group consisting of H, hydroxyl, halo, —N⁺-group; and
m is 0-30.

21. The method of claim 1, wherein R₁, R₂, R₃, R₄, R₅, and p are not simultaneously H, C₁₅ alkenyl, OH, H, H, and 13, respectively, and wherein R₁, R₂, R₃, R₄, R₅, and p are not simultaneously H, C₁₅ alkenyl or C₁₅ alkyl, OH, H, H, and O, respectively.

22. The method of claim 6, wherein R₆, R₇, R₈, R₉, R₁₀, R₁₁, R₁₂, and m are not simultaneously H, OH, H, C₁₅ alkenyl, OH, H, H and 13, respectively, and wherein R₆, R₇, R₈, R₉, R₁₀, R₁₁, R₁₂, and m are not simultaneously H, OH, H, C₁₅ alkenyl or C₁₅ alkyl, OH, H, H, and 0. respectively.

23. The method of claim 16, wherein R₆, R₇, R₈, R₉, R₁₀, R₁₁, R₁₂, and m are not simultaneously H, OH, H, C₁₅ alkenyl, OH, H, H and 13, respectively, and wherein R₆, R₇, R₈, R₉, R₁₀, R₁₁, R₁₂, and m are not simultaneously H, OH, H, C₁₅ alkenyl or C₁₅ alkyl, OH, H, H, and 0. respectively.

24. The method of claim 14, wherein the 2-hydroxyl carboxylic acid is a racemic mixture.
